(19) United States
(12) Patent Application Publication
(10) Pub. No.: US 2011/0158922 A1
(43) Pub. Date: Jun. 30, 2011

(54) Skin Care Compositions and Method of Use Thereof

(75) Inventors: Eric DUPONT, Ste-Petronille Ile d'Orleans (CA); Marc SAMSON, Quebec (CA); Alyson GALDERISI, Wayne, NJ (US)

(73) Assignee: IMMANENCE INTEGRALE DERMO CORRECTION INC., Quebec (CA)

(21) Appl. No.: 12/904,263

(22) Filed: Oct. 14, 2010

Related U.S. Application Data
(63) Continuation of application No. PCT/CA09/00494, filed on Apr. 15, 2009.
(60) Provisional application No. 61/045,114, filed on Apr. 15, 2008.

(51) Int. Cl. A61K 8/37; A61K 36/185; A61P 17/10; A61P 17/00; A61Q 19/06; A61Q 19/00; A61Q 19/08; A61P 29/00; A61P 17/08; A61Q 19/02; A61P 17/18; A61P 39/04; A61P 9/00; A61P 35/00

(52) U.S. Cl. 424/59; 424/62; 424/725

(57) ABSTRACT

A topical composition comprising at least 10 isolated active agents which each have at least one activity selected from the group consisting of anti-oxidant activity, anti-matrix metalloproteinase (MPP) activity, anti-elastase activity, anti-inflammation activity, anti-irritation activity, microcirculation support activity, collagen synthesis activity, energy production activity, oxygenation activity, DNA protection and repair activity, cell anchorage support activity, dermal-epidermal cohesion support activity, cellular renewal activity, synthesis and/or protection of glycosaminoglycans activity, prevention and/or repair and/or elimination of damaged proteins activity, protection of skin immune function activity, hydration activity, modulation of skin pigmentation activity, and anti-cellulite activity, the composition possessing at least five of the foregoing activitie. Methods of use thereof.

### CROSS REFERENCE TO RELATED APPLICATIONS

This application is a continuation application of PCT application no PCT/CA09/000,494 filed on 15 Apr. 2009 and published in English under PCT Article 21(2), which itself claims benefit of U.S. provisional application Ser. No. 61/045,114, filed on Apr. 15, 2008. All documents above are incorporated herein in their entirety by reference.

### FIELD OF THE INVENTION

The present invention relates to skin care compositions and methods of use thereof.

### BACKGROUND OF THE INVENTION

Aging is a multifactorial phenomenon. The aging of the skin is mainly the result of one's genetic predisposition (known as chronological aging) and one's physiological reaction to environmental stresses (known as actinic aging). Chronological aging is largely genetically driven and appears to be largely linked to a reduction in anti-oxidant production. Actinic aging seems to be skin specific and is defined as the effect of the external environment on the skin's biological response. The skin response to actinic aging, which may be caused by sun and pollution exposure as well as smoking, is typically associated with a lack of normal hydration, apparition of telangiectasia, sagging of the skin, and the appearance of fine lines and wrinkles.

Conventional anti-aging skin products target a single or a few causes of aging. There is a need for new improved anti-aging skin products addressing multiple causes simultaneously.

The present description refers to a number of documents, the content of which is herein incorporated by reference in their entirety.

### SUMMARY OF THE INVENTION

More specifically, in accordance with the present invention, there is provided A topical composition comprising at least 10 isolated active agents which each have at least one activity selected from the group consisting of anti-oxidant activity, anti-matrix metalloproteinase (MPP) activity, anti-elastase activity, anti-inflammation activity, anti-irritation activity, microcirculation support activity, collagen synthesis activity, energy production activity, oxygenation activity, DNA protection and repair activity, cell anchorage support activity, dermal-epidermal cohesion support activity, cellular renewal activity, synthesis and/or protection of glycosaminoglycans activity, prevention and/or repair and/or elimination of damaged proteins activity, protection of skin immune function activity, hydration activity, modulation of skin pigmentation activity, and anti-cellulite activity, the composition possessing at least five of the foregoing activities.

In a specific embodiment, the composition further comprises an acryloyldimethyltaurate derivative and an emulsion stabilizing agent, the composition being devoid of or comprising less then about 2% w/w of emulsifying agent, the emulsifying agent when present having a hydrophilic-lipophilic balance (HLB) of more than about 11. In another specific embodiment, the emulsifying agent when present has a hydrophilic-lipophilic balance (HLB) of between about 11 and 16, in a more specific embodiment between about 12 and 16, in a more specific embodiment between about 13 and 16, in a more specific embodiment between about 14 and 16, in a more specific embodiment between about 15 and 16, and in a more specific embodiment around 15.6.

In another specific embodiment, the emulsion stabilizing agent is a carbomer or a xantham gum. In another specific embodiment, the acryloyldimethyltaurate derivative is in a concentration of between about 0.2% w/w and about 5% w/w. In another specific embodiment, the acryloyldimethyltaurate derivative is in a concentration of between about 0.4% w/w and about 2% w/w. In another specific embodiment, the acryloyldimethyltaurate derivative is in a concentration of between about 0.4% w/w and about 0.75% w/w

In another specific embodiment, the acryloyldimethyltaurate derivative is ammonium acryloyldimethyltaurate derivative. In another specific embodiment, the ammonium acryloyldimethyltaurate derivative is ammonium acryloyldimethyltaurate/vinyl pyrrolidone copolymer. In another specific embodiment, the acryloyldimethyltaurate derivative is ammonium acryloyldimethyltaurate/Beheneth-25.

In another specific embodiment, the emulsion stabilizing agent is a carbomer or a xantham gum and is in a concentration of between about 0.2% w/w and about 5% w/w. In another specific embodiment, the carbomer or a xantham gum is in a concentration of between about 0.4% w/w and about 2% w/w. In another specific embodiment, the carbomer or a xantham gum is in a concentration of between about 0.8% w/w and about 1.2% w/w. In another specific embodiment, water is in a concentration of about 20% to 90% w/w.

In another specific embodiment, the composition comprises at least 11 active agents. In another specific embodiment, the composition comprises at least 12 active agents. In another specific embodiment, the composition comprises at least 13 active agents. In another specific embodiment, the composition comprises at least 14 active agents. In another specific embodiment, the composition comprises at least 15 active agents. In another specific embodiment, the composition comprises at least 16, 17, 18 or 19 active agents. In another specific embodiment, the composition comprises at least 20 active agents. In another specific embodiment, the composition comprises at least 21, 22, 23 or 24 active agents. In another specific embodiment, the composition comprises at least 25 active agents. In another specific embodiment, the composition comprises at least 26, 27, 28 or 29 active agents. In another specific embodiment, the composition comprises at least 30 active agents. In another specific embodiment, the composition comprises at least 31, 32, 33 or 34 active agents. In another specific embodiment, the composition comprises at least 35 active agents. In another specific embodiment, the composition comprises at least 36, 37, 38 or 39 active agents. In another specific embodiment, the composition comprises at least 40 active agents. In another specific embodiment, the composition comprises at least 41, 42, 43 or 44 active agents.

In another specific embodiment, the composition comprises at least six of the activities described above. In another specific embodiment, the composition comprises at least seven of the foregoing activities. In another specific embodiment, the composition comprises at least eight of the foregoing activities. In another specific embodiment, the composition comprises at least nine of the foregoing activities. In another specific embodiment, the composition comprises at least ten of the foregoing activities. In another specific embodiment, the composition comprises at least eleven of the foregoing activities. In another specific embodiment, the composition comprises at least twelve of the foregoing activities. In another specific embodiment, the composition comprises at least thirteen of the foregoing activities. In another specific embodiment, the composition comprises at least fourteen of the foregoing activities. In another specific embodiment, the composition comprises at least fifteen of the foregoing activities. In another specific embodiment, the composition comprises at least sixteen of the foregoing activities. In another specific embodiment, the composition comprises at least seventeen of the foregoing activities. In another specific embodiment, the composition comprises at least eighteen of the foregoing activities. In another specific embodiment, the composition possesses all the foregoing activities.

In another specific embodiment, each active agent is in a concentration of at least 0.0025% w/w of the composition. In another specific embodiment, the composition further comprises at least one controlled delivery system. In another specific embodiment, the composition further comprises at least one preservative. In another specific embodiment, the composition further comprises at least one chelating agent. In another specific embodiment, the composition is paraben free. In another specific embodiment, the composition further comprises at least one detackifying agent. In another specific embodiment, the composition further comprises at least one viscosity-increasing agent. In another specific embodiment, the composition further comprises at least one film forming agent. In another specific embodiment, the composition further comprises at least one fragrance. In another specific embodiment, the composition further comprises at least one anti-browning agent. In another specific embodiment, the composition further comprises at least one light-diffracting agent.

In accordance with another aspect of the present invention, there is provided a composition of the present invention for use in the reduction or prevention of at least one skin aging sign. In a specific embodiment, the skin aging sign is selected from the group consisting of fine lines, wrinkles, inflammation, redness, telangiectasia, skin sagging, excess sebum, enlarged pores, dark circles, loss of skin firmness, brown spot, dull skin, bags under eyes, disturbance of sebum production, loss of skin comfort, dehydration, and skin devitalization.

In accordance with another aspect of the present invention, there is provided a composition of the present invention for use in the reduction or prevention of at least one skin condition or disorder. In a specific embodiment, wherein the skin condition or disorder is selected from the group consisting of rosacea, acne-rosacea, spider veins, skin flushing, acne, pimples, dermatitis, rashes, irregular skin tone, cellulite, clogged pores and blotches.

In accordance with another aspect of the present invention, there is provided a use of the composition of the present invention, in the manufacture of a topical formulation.

In accordance with another aspect of the present invention, there is provided a use of the composition of the present invention, for reducing or preventing at least one skin aging sign. In a specific embodiment, the skin aging sign is selected from the group consisting of fine lines, wrinkles, inflammation, redness, telangiectasia, skin sagging, excess sebum, enlarged pores, dark circles, loss of skin firmness, brown spot, dull skin, bags under eyes, disturbance of sebum production, loss of skin comfort, dehydration, and skin devitalization.

In accordance with another aspect of the present invention, there is provided a use of the composition of the present invention, for reducing or preventing at least one skin condition or disorder. In a specific embodiment, the skin condition or disorder is selected from the group consisting of rosacea, acne-rosacea, spider veins, skin flushing, acne, pimples, dermatitis, rashes, irregular skin tone, cellulite, clogged pores and blotches.

In accordance with another aspect of the present invention, there is provided a method for preventing or reducing a skin aging sign or a skin condition or disorder in a subject comprising applying an effective amount of the composition of the present invention on the skin of the subject, whereby the skin aging sign or a skin condition or disorder is prevented or reduced.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents before and after pictures of skin treated with a composition (see Example 11) of the present invention (62 years old woman);

FIG. 2 presents before and after pictures of skin treated with a composition (see (Example 11) of the present invention (24 years old woman);

FIG. 3 presents pictures the evolution over 2 months and ½ of bags under the eyes treated with a composition (see Example 11) of the present invention; (72 years old woman);

FIG. 4 presents before and after pictures of skin treated with a composition of the present invention (see Example 11) (77 years old man); and

FIG. 5 presents the effect of a composition of the present invention (see Example 2) on skin parameters (skin texture, skin tonus, fine lines and wrinkles and pore sizes). Subjects: 20 women, age 35 to 62, Type of skin:

normal, Application: face, neck, eye, Frequency: 2×/day, Duration: 3 months; Evaluation: silicone imprints, profilometry, corneometry.

### DESCRIPTION OF ILLUSTRATIVE EMBODIMENTS

The present invention relates to compositions that possess a plurality of anti-aging activities. The following are examples of anti-aging activities possessed by specific embodiments of the compositions of the present invention. Without being so limited, in specific embodiments, the compositions of the present invention possess all the following anti-aging activities. The following lists actives and excipients that can be used in the present invention. The International Cosmetic Ingredient Dictionary and Handbook, 12th Ed, 2008, U.S. Personal Care Products Council's lists other useful ingredients for inclusion in the composition of specific embodiments of the present invention.

### Anti-Oxidants

Free radicals (FR), reactive oxygen species (ROS) and reactive nitrogen species (RNS) can cause considerable damage to the skin by attacking the cells' structure as well as components of the extracellular matrix. To fight against the deleterious effects of FR, ROS and RNS, the skin produces natural protectors known as anti-oxidants. These anti-oxidants either deactivate or scavenge FR, ROS and RNS, thereby protecting the skin against these chemical aggressors. Unfortunately, factors such as age (already possible beyond 25 years of age), exposure to UV, stress, environmental pollution, or even simply excess production of FR, ROS, or RNS weaken the natural anti-oxidant defence system, making the skin more prone to damaging oxidation reactions. Most, if not all, anti-oxidants presently used as cosmetic ingredients become inactive upon reaction with an FR, ROS, and RNS. As such, the anti-oxidant potential of cosmetic formulation may vanish rapidly. In specific embodiments, the present invention uses ethylbisiminomethylguaiacol manganese chloride a recently developed anti-oxidant technology that inactivates and scavenges FR, ROS and RNS and self-regenerates upon completion of the chemical reactions involved. This technology allows the anti-oxidant potential of the cosmetic formulation to recycle itself and to remain active for extended periods of time.

The terms “anti-oxidant agent” as used herein is meant to refer to any ingredient capable of eliminating or reducing oxidative reactions in skin and/or in the cosmetic formulation itself. Without being so limited, the following are examples of ingredients that may act as anti-oxidants in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such ingredients include ethylbisiminomethylguaiacol manganese chloride; blend of dipalmitoyl hydroxyproline, dimethylmethoxy chromanol; hesperetin laurate, blend of hesperidin methyl chalcone, steareth 20, dipeptide-2 (dipeptide Valyl-tryptophane), and palmitoyl tetrapeptide-7, olive leaf extract, ubiquinone, super-oxide dismutase, flavanols, isoflavones, furfuryladenine, panthenol, lipoic acid, niacinamide, melanin, catalase, glutathione, polyphenols, cysteine, allantoin, kinetin, ascorbic acid and its derivatives (ascorbyl palmitate, magnesium ascorbyl phosphate, sodium ascorbyl phosphate), vitamin E and its derivatives (e.g., α-tocopherol, δ-tocopherol, γ-tocopherol, tocopheryl acetate), squalane, lipochroman-6, licorice extract, grape seed extract, and camellia sinensis extract. See also The International Cosmetic Ingredient Dictionary and Handbook, 12th Ed, 2008, U.S. Personal Care Products Council's for other anti-oxidant agents.

### Anti-MMPs (Collagen Protection)

MMPs (matrix metalloproteinases) are natural skin enzymes that are involved in the slow turnover of collagen fibers. In the skin, the presence of natural inhibitors (neutralizers) of MMPs prevents excessive activity of MMPs. The delicate equilibrium between MMPs and their inhibitors ensures the homeostasis of the extracellular matrix allowing the skin to maintain its firmness and healthy look. However, this delicate balance between MMPs and their inhibitors can be upset under certain conditions. Aging (already possible past 25 years of age), UV exposure, stress, cigarette smoke and environmental pollution are known to shift this enzymatic balance in favor of excessive MMP activity. This phenomenon will eventually lead to an increased breakdown of collagen fibers and a progressive dismantlement of the extracellular matrix.

In specific embodiments, the present invention contains a marine-derived ingredient that is a powerful natural inhibitor of MMPs, namely glycosaminoglycans. This active agent, a result of leading-edge biotechnology, “replenishes” the skin with natural MMP inhibitors and re-establishes the enzymatic equilibrium. The maintenance and the re-establishment of the integrity of the extracellular matrix of the skin largely contribute to integral dermo-correction. Without being so limited, useful glycosaminoglycans for use in the present invention are extracted from shark cartilage and are described in U.S. Pat. Nos. 5,618,925 issued Apr. 8, 1997; 5,985,839 issued Nov. 16, 1999; 6,025,334 issued Feb. 15, 2000; 6,028,118 issued Feb. 22, 2000; and 6,635,285 issued Oct. 21, 2003 to Aeterna Zentaris GmbH.

Without being so limited, the following are other active agents that may inhibit MMPs in the composition of the present invention: plant extracts (i.e. fruit, vegetable and/or leguminous, flower, and/or spice extracts), algae extracts, microorganisms extracts such as yeast extracts and their derivatives, ferments, proteolytic hydrolysates, peptides, animal derivative extracts, including shark cartilage extracts, and synthetic compounds. More particularly, such active agents include adenosine, camellia sinensis extract, polyphenols, ubiquinone, spatholobi caulis extract, euonymus alatus extract, rhizoma notopterygii extract, quercetin, glycosaminoglycans, palmitoyl pentapeptide-4, polymethoxy flavonoid, licorice extract, N-acetyl-cysteine, 2-furildioxime, isoflavone, vitamin C and its derivatives (ascorbyl palmitate, magnesium ascorbyl phosphate, sodium ascorbyl phosphate), retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, γ-carotene, retinyl ester), hydroxamate derivatives, glycosaminoglycans, APT™ (Ahnfeltia concinna extract), alpha2 adrenergic receptor agonist (US20090060852), sodium chondroitin sulfate, caffeine, tetrahydrozoline hydrochloride and pueraria lobata root extract. See also The International Cosmetic Ingredient Dictionary and Handbook, 12th Ed, 2008, U.S. Personal Care Products Council's for other MMPs inhibitors.

### Anti-Elastase (Elastin Protection)

Elastase is an enzyme that attacks and destroys elastin fibers, an important component of the extracellular matrix. Elastin fibers confer resiliency and visco-elastic properties to the skin. Aging is responsible for an increased activity of the enzyme elastase leading to an excessive breakdown of elastin fibers. This results in the progressive loss of the visco-elastic properties of the skin. In specific embodiments, the present invention contains an inhibitor of the enzymatic activity of elastase. This action prevents excessive degradation of elastin and thus helps maintain the resiliency and the visco-elasticity of the skin.

Without being so limited, the following are examples of active agents that may inhibit or reduce elastase activity in the composition of the present invention: plant extracts (i.e. fruit, vegetable and/or leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include hesperetin laurate; blend of hesperidin methyl chalcone, steareth 20, dipeptide-2, and palmitoyl tetrapeptide-7; soy extracts, malt extract, ursolic acid, dipalmitoyl hydroxyproline, and solanacear extract. See also The International Cosmetic Ingredient Dictionary and Handbook, 12th Ed, 2008, U.S. Personal Care Products Council's for other active agents that reduce elastase activity.

### Anti-Inflammation

With aging including photoaging, the skin is known to become more susceptible to inflammation. Inflammation is a defence mechanism for the skin against various forms of attack: physical, chemical, and biological. During inflammation, proteolytic activity is increased around microvessel walls, facilitating the recruitment and migration of pro-inflammatory cells in the surrounding tissues. The migration process itself activates such cells to release additional proteolytic enzymes, as well as inflammatory cytokines and lipids. The increased degradation of collagen by MMPs and elastin by elastase, that ensues, leads to the generation of fragments that further contribute to recruit pro-inflammatory cells in a vicious circle, accelerating the aging of the skin in the process. In specific embodiments, the present invention contains at least one anti-inflammatory agent, i.e. active agents able to slow down the process of activating pro-inflammatory cytokines and lipids.

In a specific embodiment, the present invention may contain for instance lyophilized hesperetin laurate, a bioflavonoid. The process of lyophilisation increases the stability of the flavonoid molecules while maintaining optimal biological activity. The present invention encompasses the use of lyophilized active agents to increase their stability. These bioflavonoids have the ability to reduce micro-vessel permeability. This is most likely mediated through demonstrated free radical scavenging and anti-elastinolytic effects. Maintaining ECM integrity in the surrounding of micro-vessels is associated with a straightening of microvessel walls and a reduction in their permeability. As a consequence, recruitment and activation of pro-inflammatory cells is reduced within skin tissues.

Without being so limited, the following are examples of active agents that may inhibit or reduce inflammation in the composition of the present invention: plant extracts (i.e. fruit, vegetable and/or leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include allantoin, ubiquinone, ethylbisiminomethylguaiacol manganese chloride, vitamin E and its derivatives (e.g. α-tocopherol, δ-tocopherol, γ-tocopherol, tocopheryl acetate), chamomile oil, gingko biloba oil, camellia sinensis extract, beta-glucans, bisabolol, zea mays (corn) extract, licorice extract, Chinese kudzu; azelaic acid; glycosaminoglycans; blend of capryl/capric succinic triglyceride, sesamum indicum seed oil, triticum vulgare germ oil, and tocopheryl acetate; dipalmitoyl hydroxyproline; hesperetin laurate; palmytoyl tetrapeptide-7, and palmitoyl tripeptide-8. See also The International Cosmetic Ingredient Dictionary and Handbook, 12th Ed, 2008, U.S. Personal Care Products Council's for other active agents that may inhibit or reduce inflammation.

### Anti-Irritation

When the skin is exposed to environmental stresses such as UV radiation, chemicals, and pollution, an inflammatory response may occur. The formation of so-called pro-inflammatory cytokines and prostaglandins (lipids) initiates a cascade of reactions that can ultimately accelerate skin aging and photoaging.

In order to counteract irritation and inflammation, specific embodiments of the present invention also contain at least one anti-irritation agent. In a specific embodiment, the present invention contains an anti-irritation agent derived from Alteromonas, a microorganism found in hydrothermal deep vents. This microorganism secretes functional polysaccharides most likely acting as a biological shield. A biotechnological extraction process has been developed to extract the polysaccharide complex that composes this anti-irritation agent.

In another specific embodiment, the present invention contains an anti-irritation agent consisting of a complex of fatty acids, oils with cosmetic applications and liposoluble vitamins. This complex may bring hydrating and anti-inflammatory effects to the skin. The active agents of this complex can improve the skin barrier function and protect against environmental assaults.

Without being so limited, the following are examples of active agents that may reduce irritation in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include allantoin, camellia sinensis extract, lavender oil, aloe vera, linden extract, epilobium angustifolium extract, chysanthellum indicum extract, cola nitida extract, Alteromonas ferment extract, beta-glucans, bisabolol, licorice extract, mallow extract, centella asiatica, and blend of capryl/capric succinic triglyceride, sesamum indicum seed oil, triticum vulgare germ oil, tocopheryl acetate. See also The International Cosmetic Ingredient Dictionary and Handbook, 12th Ed, 2008, U.S. Personal Care Products Council's for other active agents that may reduce irritation.

Other specific embodiments may also contain a system that allows a delayed release of at least one of the active agents in the upper layers of the skin.

In a specific embodiment, the composition of the present invention uses PEG-8/SMDI as controlled delivery system. With this compound, non encapsulated active agents find an efficient vehicle for proper dispersion, thereby allowing improved bioavailability and slow release over time.

Without being so limited, the following are examples of active agents that may allow delayed release of active agents in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include dextrin, cyclodextrin, 7-Dehydrocholesterol (cholesterol precursor), maltodextrin and PEG-8/SMDI. See also The International Cosmetic Ingredient Dictionary and Handbook, 12th Ed, 2008, U.S. Personal Care Products Council's for other active agents that may allow delayed release of active agents.

### Microcirculation Support

The increase release of pro-inflammatory mediators, proteolytic activity, and ROS, FR and RNS seen with aging, including photoaging, ultimately affects skin microcirculation. The three factors converge to alter the structural integrity of dermal microcapillaries and lymphatic vessels, causing vasodilatation and increased vessel permeability. As a consequence, plasmatic fluids tend to leak within the dermis and generate edema. Importantly, localized fluid retention is responsible for the appearance of puffiness (bags) and dark circles around eyes. Vasodilatation is also associated with rosacea, telangiectasia, redness, and flushing, which are cosmetic concerns in progression with aging.

In specific embodiments, the present invention seeks to improve the structural integrity of dermal microcapillaries and lymphatic vessels by supporting matrix integrity. In specific embodiments, one particular active agent, namely glycosaminoglycans (GAGs), is aimed directly at this aspect. GAGs inhibit the MMPs proteolytic activity that contributes to the deterioration of the extracellular matrix around microcapillaries and lymphatic vessels. GAGs also have anti-inflammatory and anti-oxidant effects that further protect the structural integrity of dermal microcapillaries and lymphatic vessels.

In specific embodiments, the present invention may also contain at least one agent for reducing or preventing dark circles and eye-puffiness. In a specific embodiment, the present invention may contain a blend of hesperidin methyl chalcone, steareth 20, dipeptide-2, and palmitoyl tetrapeptide-7 which has been shown to significantly reduce eye puffiness clinical settings.

Without being so limited, the following are examples of active agents that may support microcirculation and reduce eye puffiness and dark circles, in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include glycosaminoglycans, hesperetin laurate, blend of hesperidin methyl chalcone, steareth 20, dipeptide-2, and palmitoyl tetrapeptide-7; mixture of pseudoalteromonas ferment extract, hydrolyzed wheat protein, hydrolyzed soy protein, tripeptide-10 citrulline, and tripeptide-1; blend of hydrolyzed rice bran protein, glycine soja (soybean) protein and oxido reductases; butchersbroom extract, proline, caffeine, soy extracts, acetyl tetrapeptide 5, mixture of ascophyllum nodosum extract and asparagopsis armata extract; and sodium ascorbyl phosphate (SAP). See also The International Cosmetic Ingredient Dictionary and Handbook, 12th Ed, 2008, U.S. Personal Care Products Council's for other active agents that may support microcirculation and may therefore reduce dark circles and/or eye puffiness.

### Collagen Synthesis

Collagen fibers are the main structural components of the dermis. They are mandatory for the skin extracellular matrix to maintain its resiliency and tri-dimensional organization. Collagen fibers provide cellular support and also anchoring fibrils sitting at the dermal-epidermal interface. Collagen components are synthesized and assembled within fibroblasts, then secreted into the extracellular media where they bundle. Collagen fibers are normally subjected to a slow turnover. Their degradation by MMPs releases small fragments that are sensed by skin fibroblasts and interpreted as a signal for the need for new synthesis. However, aged fibroblasts have a reduced capacity for synthesis and may need assistance to regenerate collagen.

In specific embodiments, the present invention may contain at least one agent for the stimulation of collagen synthesis. In other specific embodiments, the present invention contains the peptide palmitoyl-pentapeptide 4 (Palmitoyl-Lysysl-Threonyl-Threonyl-Lysyl-Serine (SEQ ID NO: 1)). This peptide reinforces the capacity of skin fibroblasts to synthesize collagen fibers by acting through a physiological “feedback” pathway that stimulates cell activity. The action of palmitoyl-pentapeptide 4 mimics a positive feedback that already exists in the skin. Palmitoyl-pentapeptide 4, to some extent, mimics the action of the small fragments of collagen mentioned above. Palmitoyl-pentapeptide 4 promotes skin firmness through a natural physiological process.

Without being so limited, the following are examples of active agents that may stimulate collagen synthesis in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include adenosine, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin C and its derivatives (ascorbyl palmitate, magnesium ascorbyl phosphate, sodium ascorbyl phosphate), growth factors and their derivatives, palmitoyl pentapeptide-4, acetyl octapeptide-3, Alteromonas ferment extract, palmitoyl tripeptide-5, caprooyl tetrapeptide-3, and malt extract.

### Energy Production

Skin cells need energy to remain metabolically active and to perform vital functions. Within cells, mitochondria are little bean-shaped organelles that couple cellular respiration with energy production which is stored under the form of ATP (adenosine triphosphate)-creatine with the help of creatine kinase enzymes. The energy banked in these links is released upon ATP consumption and serves to support cellular metabolism. Accumulation of ROS damages with aging alters mitochondrial integrity and reduces creatine kinase activity, thus affecting cell metabolic activity and leading to cellular senescence. Additional ROS are produced as by-products of an impaired mitochondrial respiration. Old mitochondria produce less ATP causing an energy crisis that, at the skin level, translates into a dull complexion.

In specific embodiments, the present invention may contain at least one agent that can regenerate the pool of ATP through the donation of PO₄ residue. In this way, skin cells have access to renewed bioenergy and can normally carry on the normal metabolic functions that are necessary to maintain skin homeostasis.

In a more specific embodiment, the present invention contains creatine. Studies on creatine used in specific embodiments of the present invention show a substantial improvement in skin cell energy production upon topical application.

Without being so limited, the following are examples of active agents that may regenerate/stimulate ATP in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include creatine, seanergilium BG (brown algae), esculoside, and carnitine. See also The International Cosmetic Ingredient Dictionary and Handbook, 12th Ed, 2008, U.S. Personal Care Products Council's for other active agents that may regenerate/stimulate ATP.

### Oxygenation

Oxygen supplementation to the skin is assured in two complementary ways, one internal and one external. Within the body, blood carries oxygen which is delivered to cells through diffusion, following its release from the hemoglobin content of red cells. There are no blood vessels within the epidermis. As a consequence, oxygen has to diffuse from the small vessels that irrigate the dermis to the upper layers. Unfortunately, dermal capillaries become more fragile with aging and oxygen diffuses less efficiently from the deeper to the upper layers of the skin. From external sources, oxygen is absorbed through cellular respiration which is influenced by cellular metabolism and the quality of ambient air. Over time, urban style living can contribute to poor oxidation of skin cells, therefore negatively affecting the complexion of the skin.

In specific embodiments, the present invention may contain at least one agent that promotes or facilitates the delivery or the creation of oxygen molecules directly into skin.

In a specific embodiment, the present invention uses ethylbisiminomethylguaiacol manganese chloride (a salen-manganese compound) as oxygenation agent. The catalytic site of this compound is a Mn atom. When activated as described in the series of reactions described above, this compound is quite unstable and needs to rapidly react with a final peroxyde molecule to form water and oxygen molecules. In this reaction, Mn returns to a redox state of III and is ready to undertake another complete cycle of free radical/ROS elimination. The molecule is said to be self-regenerating.

The following reaction flow chart demonstrates how molecular oxygen can be created in situ by salen-manganese compounds such as ethylbisiminomethylguaiacol manganese chloride:

### DISMUTATION OF O₂—

Mn(III)+O₂—→Mn(II)+O₂ is the reduction of Mn (III) to Mn (II)
2H+ +Mn(II)+O₂—→Mn(III)+H₂O₂ is Mn (II) oxidized to Mn (III)

### SCAVENGING OF H₂O₂

Mn(III)+H₂O₂→Mn(V)O₂—+H₂O is Mn (III) oxidized into oxoMn-salen by H₂O₂
Mn(V)O2-+H₂O₂→Mn(III)+H₂O+O₂ is oxoMn-salen reduced to Mn (III)

### SOD Reaction (Dismutation of Superoxide)

In its original form, the Mn atom exists in a valence, or redox state, of III. Upon reaction with a superoxide anion, the Mn atom is reduced to a redox state of II by the free electron of the free radical. The Mn (II) then reacts with a second superoxide (and with protons) and is re-oxidized back to redox (III). In this reaction, hydrogen peroxyde, the normal product of the SOD reaction, is generated.

### Catalase Reaction (Scavenging of Hydrogen Peroxyde).

Mn (III) reacts with a molecule of hydrogen peroxyde and becomes oxidized to an oxoMn-salen with a redox state of V. The latter further reacts with H₂O₂ being reduced back to Mn (III). In the course of this reaction, water and molecular oxygen are generated.

Without being so limited, the following are active agents that may promote skin oxygenation in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include ethylbisiminomethylguaiacol manganese chloride, placenta enzymes, glycoproteins, squalane, ubiquinone, and dimethyl methoxy chromanol. See also The International Cosmetic Ingredient Dictionary and Handbook, 12th Ed, 2008, U.S. Personal Care Products Council's for other active agents that may promote skin oxygenation.

### DNA Protection and Repair

DNA, the very heart of cells, holds the genetic code that dictates the linear and tri-dimensional structure of proteins. Oxidative reactions that increase with aging and sun exposure affect DNA, generating damages that prevent its exact replication during cell division or introducing mutations that precipitate the process of aging. ROS-induced DNA damages are of various types. DNA base oxidation, thymidine dimmer (T-T) formation, and DNA strand breaks are commonly seen following UV exposure and are associated with skin photoaging. It is estimated that there are thousands of DNA alterations rising in each cell daily. Fortunately enough, the skin has developed mechanisms to repair these damages such as mismatch repair, nucleotide excision repair, and double-strand break repair that operate through various enzymes. Sirtuins are a class of enzymes involved in DNA maintenance. Sirtuins are protein deacetylases. Some of their targets are histone proteins which upon deacetylation are free to bind DNA molecule, stabilizing DNA long enough to allow double strand break repair. However, not surprisingly, the efficiency of DNA repair systems diminishes with time and sun exposure a phenomenon associated with accelerate aging.

In a specific embodiment, the present invention may contain at least one active agent that protects DNA against UV-induced damage, thereby facilitating any eventual repair.

In a more specific embodiment, ethylbisiminomethylguaiacol manganese chloride plays this role in the composition of the present invention. In vitro studies on ethylbisiminomethylguaiacol manganese have shown that the molecule reduces the formation of T-T dimmers in DNA and stimulates DNA repair, thus increasing cell viability following UV exposure.

In another specific embodiment, Oryza sativa (rice) extract plays this role in the composition of the present invention. Oryza sativa is an activator of Sirtuins expression and activity that improves skin protection and repair after UV and oxidative damage.

Without being so limited, the following are examples of active agents that may promote DNA protection and repair in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include ethylbisiminomethylguaiacol manganese chloride, oryza sativa extract, AC-11™ (Uncaria tomentosa), buddleja davidii extract, citrullus lanatus fruit extract, resveratrol, creatine, and ubiquinone.

### Support of Cell Anchorage

Skin aging is accompanied by a progressive disorganization of the extracellular matrix (ECM) within the dermis that generates wrinkles and sagging. Dermal ECM is made up of collagen, elastin, proteoglycans, and GAGs that together contribute to establish the structural integrity of the skin. In the dermis, ECM components are produced by fibroblasts to form a tridimensional network that, in return, provides support and anchorage for cells. Proper attachment of dermal fibroblasts to the ECM promotes cell functions, including migration, proliferation, and differentiation. Thus, not surprisingly, any modification in the dermal ECM tridimensional network is susceptible of contributing to skin aging by affecting the biomechanical properties of skin.

In a specific embodiment, the present invention may contain at least one active agent that promotes fibroblast activity. In a more specific embodiment, at least dipalmitoyl hydroxyproline plays this role in the composition of the present invention. Dipalmitoyl hydroxyproline possesses multiple properties that translate into an increased ability of fibroblasts to anchor to and support the collagen network. This phenomenon might have a positive effect on the tri-dimensional integrity of the ECM.

Without being so limited, the following are examples of active agents that may promote fibroblast activity through improved cell anchorage, in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include dipalmitoyl hydroxyproline, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin D and its derivatives (cholecalciferol, ergocalciferol, 25-hydroxycholecalciferol), growth factors, estradiol derivatives, alpha hydroxy acids, tripeptide-10 citrulline, polysaccharides, beta-glucans, Ahnfeltia concinna extract (APT™), salicilyc acid (e.g. willow bark extract); Anemarrhenae asphodeloides root extract; Acetyl tetrapeptide-2; Ascophyllum nodosum extract; ubiquinone (Coenzyme q10); Acetyl octapeptide-3; Caprooyl tetrapeptide-3; glycolic acid; lactic acid; citrus acid and walnut shell powder.

### Dermal-Epidermal Cohesion

One of the most striking manifestations of skin aging, as revealed by histological techniques, is a flattening at the dermo-epidermal junction (DEJ) with loss of the dermal papillae. The latter forms villosities that facilitate nutritional exchanges and metabolic byproducts evacuation between the dermis and the epidermis. Their disappearance with aging contributes to slow down epidermal cell turnover. The DEJ is a structure of major importance for skin cohesion, since it anchors the epidermis to the underlying dermis. This zone is constituted by various types of anchoring fibrils such as fibronectin, laminin, collagen VII, and collagen IV. The latter is an exclusive member of the basement membranes, whose structure forms supramolecular networks that influence cell adhesion, migration, and differentiation. Collagen IV is essential for the mechanical stability of skin. Studies have shown that collagen IV content decreases with age after 35 years, weakening skin structure and contributing to wrinkle formation.

In a specific embodiment, the present invention may contain at least one active agent that promotes skin cohesion. In a more specific embodiment, at least palmitoyl pentapeptide-4 plays this role in the composition of the present invention. Palmitoyl pentapeptide-4 is derived from a collagen precursor that cells perceive as a sign of excessive ECM degradation. The peptide triggers a positive feedback within the skin, promoting synthesis of collagen IV and fibronectin at the DEJ.

Without being so limited, the following are active agents that may promote skin cohesion in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include palmitoyl pentapeptide-4, caprooyl tetrapeptide-3, mixture of palmitoyl dipeptide-5 diaminobutyloyl hydroxythreonine and palmitoyl dipeptide-6 diaminohydroxybutyrate, Saccharomyces cerevisiae yeast extract, Cyperus esculentus (tigernut) extract, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester).

### Stimulation of Cellular Renewal

Skin renewal begins with the generation of new keratinocytes from stem cells residing in the stratum basale, the inner layer of the epidermis. As new cells keep forming, keratinocytes migrate upward and differentiate into corneocytes. Keratin, which is a highly fibrous protein, is produced during the differentiation process and causes cell walls to harden, forming the stratum corneum (SC). The terminal differentiation of keratinocytes ultimately results in cell death. Old corneocytes are shed from the SC through desquamation. Aging is associated with reduced epidermal proliferation.

In a specific embodiment, the present invention may contain at least one active agent that promotes cellular renewal. In a more specific embodiment, at least retinol plays this role in the composition of the present invention. Retinol simultaneously stimulates cell renewal at the basal layer within the epidermis, as well as differentiation as cells migrate upward to the SC. Thus Retinol facilitates skin renewal.

Unlike many retinol-containing products already on the market, specific embodiments of the present invention cause no irritation. Anti-irritation agents as well as a delayed-release system allow the skin to benefit from the retinol advantage while keeping skin smooth and soft.

Without being so limited, the following are examples of active agents that may promote cellular renewal in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include adenosine riboside and its derivatives, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), vitamin D and its derivatives (cholecalciferol, ergocalciferol, 25-hydroxycholecalciferol), growth factors, estradiol derivatives, alpha hydroxy acids, tripeptide-10 citruline, polysaccharides, beta-glucans, Ahnfeltia concinna extract (APT™), salicilyc acid (e.g. willow bark extract), Anemarrhenae asphodeloides root extract, acetyl tetrapeptide-2, Ascophyllum nodosum extract, ubiquinone, creatine, Acetyl octapeptide-3, glycolic acid, lactic acid, citrus acid, and walnut shell powder, and a phytocomplex blend of horsetail, myrrh, weath germ, and hops extracts. See also The International Cosmetic Ingredient Dictionary and Handbook, 12th Ed, 2008, U.S. Personal Care Products Council's for other active agents that may promote cell renewal.

Synthesis and/or Protection of Glycosaminoglycans

Glycosaminoglycans (GAGs) are long unbranched polysaccharides. The specific GAGs of physiological significance are hyaluronic acid, dermatan sulfate, chondroitin sulfate, heparin, heparan sulfate, and keratan sulfate. The spatial arrangement of the collagen network depends on the presence of these supporting macromolecules. In a young skins, collagen fibers are held in place by orderly bonds, to form a sort of net, within which the intercellular spaces “empty space between fibers” are filled by proteoglycans (protein-linked GAGs) and GAGs. The latters form a water-saturated gel in which water-soluble molecules and ions are able to circulate. This cutaneous intercellular fluidic network of macromolecules plays an important role in the tri-dimensional organization of the extracellular matrix as it intermingles with collagen fibers. Aging, including photoaging is associated with a loss of GAGs within the epidermis due to reduced synthesis and accelerated breakdown by hyaluronidase enzymes. Increased production and protection of GAGs help improve the integrity of the extracellular matrix and support skin hydration.

In a specific embodiment, the present invention may contain at least one active agent that promotes GAGs synthesis. In a more specific embodiment, marine GAGs play this role. GAGs macromolecules can hold up to 1000 times their weight in water, making them key components for skin hydration, as a complementary way to support moisturizing action of other actives that are encompassed in specific embodiments of the present invention. Moreover, this fluidity feature given to the extracellular matrix favors intercellular migration of growth factor signals and of essential micronutrients.

In another specific embodiment, at least palmitoyl pentapeptide-4 plays this role in the composition of the present invention. Palmitoyl pentapeptide-4 was shown in an in-vitro study to increase synthesis of GAGs.

Without being so limited, the following are examples of active agents that may promote GAGs synthesis and/or protection in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include palmitoyl pentapeptide-4, acetyl hexapeptide-3, ahnfeltia concinna extract (APT™), theophylline, quercetin, kaempferol, licorice extract (as an inhibitor of hyaluronidase), retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), and growth factors. See also The International Cosmetic Ingredient Dictionary and Handbook, 12th Ed, 2008, U.S. Personal Care Products Council's for other active agents that may promote glycosaminoglycans synthesis and/or protection.

Prevention, Repair and/or Elimination of Damaged Proteins

The extracellular matrix is rich in long-lived proteins such as collagen and elastin fibers. As such, they are vulnerable to various post-translational modifications that tend to accumulate with time and UV exposure, affecting their structure and biological functions. Among these modifications, glycation is known to affect skin proteins during aging and photoaging. Glycation results from the non-enzymatic addition of sugars to proteins, which induces abnormal protein crosslinking. The process is accelerated in the presence of high glucose levels, FR, ROS and RNS. In the aging body, ECM cross-linking contributes to hardening and brittleness of the skin, and interferes with skin renewal.

Isomerization of aspartic acid and deamidation of asparagine residues represent another significant part of the spontaneous damage to skin proteins that results from the aging and photoaging processes. These modifications happen spontaneously with time and are precipitated by UV, free radicals, ROS, and RNS exposure. Such modifications are known to affect ECM proteins such as collagen, elastin, and fibronectin, altering their functions. That type of protein damage can be at least partly repaired by an enzyme called protein isoaspartyl methyltransferase (PIMT) which is present in skin cells. However the enzyme may become overwhelmed as damages accumulate with aging. Supporting or providing additional PIMT activity, either directly or indirectly, to skin cells may help repair damaged proteins and slow the aging process in skin.

Another deleterious effect of UV, free radicals, ROS, and RNS exposure during aging is the accumulation of oxidized proteins, misfolded proteins, and protein aggregates within skin cells. The faith of these damaged proteins is normally to be degraded by the proteasome, a large protein complex found in the cytosolic and nuclear compartments of cells. During aging, however, proteasome activity declines. The accumulation of oxidized proteins that ensues is associated with decreased protein turnover in senescent fibroblasts caused by a declined in protein synthesis and proteolysis. Supporting proteasome activity is associated with an improvement in signs of aging.

In a specific embodiments, the present invention may contain at least one active agent that promotes skin prevention, repair and/or elimination of damaged proteins.

In a more specific embodiment, bioflavonoid hesperidin plays this role in the composition of the present invention. Hesperidin flavonoids were reported to inhibit collagen glycation in vitro.

In another specific embodiment, the present invention contains association of aminoguanidine hydrochloride, with either chlorogenic acids or pueraria lobata root extract which were shown to synergistically protect skin proteins form glycation activity in human skin explants ex vivo.

In another specific embodiment, the present invention contains retinol or its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester) known to stimulate proteosomal activity and promote elimination of damaged proteins in the skin.

Without being so limited, the following are examples of active agents that may promote prevention, repair or elimination of damaged proteins in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, glycation inhibitors include (but are not limited to) association of aminoguanidine, chlorogenic acids or pueraria lobata root extract; lipochroman-6; resveratrol; quercetin; arbutin; carnosine; dipeptide-4; complex of Pseudoaltermonas ferment extract, hydrolyzed wheat protein, hydrolyzed soy protein, tripeptide-10 citrulline, tripeptide-1. Supporters of PIMT activity include (but are not limited to) hydroxytyrosol, lotus extract, and S-adenosylmethionine salts. Supporters of proteasome activity include (but are not limited to) retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), and carnosine.

### Protection of Skin Immune Functions

UV radiation is one of the most significant environmental stress to which the skin is exposed. The skin exerts a barrier function that prevents foreign bodies from penetrating but also possesses an important immunological function. Langerhans cells are resident cells of the epidermis. Also known as “immune” cells of the skin, their role is to present antigens (particles from non-self molecules) to the immune system that will then deploy an immunological attack protecting the host. With aging and upon exposure to UV, Langerhans cells are depleted from the epidermis. Langerhans cell functions being lost, skin's immune defences are importantly reduced. This phenomenon is known as age- and UV-induced immunosuppression. In this case, skin becomes more susceptible to viral infection and skin cancer. Recently, the measurement of UV-induced immunosuppression is increasingly used as a functional parameter to evaluate the efficacy of sunscreens.

In a specific embodiment, the present invention may contain at least one active agent that promotes Langherhans cell protection.

In a more specific embodiment, at least Alteromonas ferment extract plays this role in the composition of the present invention.

This extract is a natural polysaccharide complex obtained through the fermentation process of Alteromonas microorganisms. This polysaccharide complex has the ability to modulate the immune reaction by protecting Langerhans cells from UV stress. The complex was shown to maintain normal Langerhans cellular density upon exposure to UV.

Without being so limited, the following are examples of active agents that may support skin immune functions in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include phytosphingosines, topical steroids, antimetabolites, vinca alkaloids, beta glucan, retinol, hyaluronic acid, salicylic acid, willow bark extract, licorice extract, and Alteromonas ferment extract. See also The International Cosmetic Ingredient Dictionary and Handbook, 12th Ed, 2008, U.S. Personal Care Products Council's for other active agents that promote cellular renewal.

### Hydration

Skin dehydration occurs in all skin types and threatens the whole skin integrity, especially in the detrimental modern air environment. A major cause of dehydration is Transcutaneous Water Evaporative Loss (TWEL) that occurs when, for instance, the skin barrier function (i.e. fluid protection inside the superior layer of skin) is insufficient or dysfunctional.

Skin moisturization is in part due to a proper water-and-osmotic equilibrium. The double lipid layer that acts as an impermeable barrier to most polar substances is an obstacle to the migration of the ions which are involved in maintaining cellular osmolarity and, in particular, in maintaining the ion concentration gradients. The transportation of inorganic ions and small molecules involves transmembrane proteins, each of which handles a specific ion or type of molecule. Selective permeability makes it possible to create considerable differences in concentrations between that of the cytoplasm and that of the extracellular medium. There is an equilibrium between the total cation concentrations inside and outside the cell, in healthy skin. These concentration differences are maintained by an Na+/K+ (sodium and potassium) pump which actively pumps K+ (potassium) into the cells and expels Na+ (sodium), thus allowing it to regulate the osmotic pressures which control the volume of the cell. To work properly, this pump must be activated by an influx of extracellular K+ (potassium) into the cell. ATP supplies the energy required for pumping the ions (via a membrane ATPase). If the influx of K+ is blocked by ouabaine (which blocks the K+ binding site), the dephosphorylation which normally induces a change in the conformation of the ‘pump’ protein, can no longer occur, and the pump stops working. The ionic gradient is no longer maintained and the cell is no longer able to control its osmotic equilibrium.

In specific embodiments, the composition of the present invention contains at least one hydrating agents that works in multiple ways to bring a maximum of hydration and comfort to the epidermis.

In addition to control hydration at the level of the epidermis, specific embodiments of the composition of the present invention may control hydration at the cellular level, and protect osmosis.

In a more specific embodiment, the composition of the present invention contains an Imperata cylindrica (luffa) root extract. This subtropical plant is able to survive for weeks without water supply because of its ability to retain water through various metabolic pathways. Imperata cylindrica extract helps skin hydration in two ways: by providing potassium essential to the osmotic balance and by delivering a specific “osmolyte” that enables skin cells to trap water molecules.

Without being so limited, the following are examples of active agents that may promote hydration in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include cucumber extract, sodium-2-pyrrolidone carboxylate, sodium PCA, sodium hyaluronate, chitin and its derivatives, alpha hydroxy acids, hyaluronic acid, hydrolysed wheat protein, a phytocomplex blend of horsetail, myrrh, weath germ, and hops extracts; glycerine, dipalmitoyl hydroxyproline, tocopheryl acetate (vitamine), dipotassium glycyrrhizate; blend of capryl/capric succinic triglyceride, sesamum indicum seed oil, triticum vulgare germ oil, and tocopheryl acetate (LNST); squalane; Imperata cylindrica root extract; blend of ceramide 3, 6 II, 1 and phytosphingosine; sodium DNA, mannitol, dulcitol, betain, di-benzo-p-dioxine (WO2009017369) and marine glycosaminoglycans. See also The International Cosmetic Ingredient Dictionary and Handbook, 12th Ed, 2008, U.S. Personal Care Products Council's for other active agents that may promote hydration.

### Skin Pigmentation

Skin pigmentation is due to the presence of melanine, a pigment produced in the epidermis by the hydroxylation of tyrosine by the enzyme tyrosinase. Melanine pigments are made in specialized cells called melanocytes, packed in organelles called melanosomes that are transferred to keratinocytes to assure proper diffusion throughout the epidermis. The terms “whitening/pigmentation agents” refer to active agents that are able to reduce or modulate skin pigmentation by limiting melanin production, inhibiting melanosome maturation and transfer, or by stimulating pigment degradation.

In a specific embodiment, the present invention may contain at least one active agent that reduces or modulates skin pigmentation.

In a more specific embodiment, at least an extract of Rumex occidentalis plays this role in the composition of the present invention. Rumex occidentalis is a plant native to the northern Canadian prairies region from which an inhibitor of tyrosinase activity has been extracted.

In another specific embodiment, at least an extract of Pisum sativum plays this role. Pisum sativum is an inhibitor of tyrosinase activity which additionally interferes with the maturation of melanosomes in melanocytes.

In another specific embodiment, retinol plays this role. Retinol is an inhibitor of tyrosinase activity which additionally promotes a decrease in the transfer of melanosomes from melanocytes to keratinocytes.

Without being so limited, the following are active agents that may modulate skin pigmentation in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, tyrosinase inhibitors include (but are not limited to) arbutin, azealeic acid, vitamin C and its derivatives (ascorbyl palmitate, magnesium ascorbyl phosphate, sodium ascorbyl phosphate), hydroquinone, N-acetyl-4-cysteanimylphenol, kojic acid, nanopeptide-1, tretinoin, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), Rumex occidentalis extract, turmeric, licorice extract, mulberry, arctostaphylos uva ursi (bearberry), mixture of ceramide 3, 6, II, 1 and phytosphingosine; acetyl octapeptide-3; sodium DNA, leukocyte extract, magnesium ascorbyl phosphate, arctostaphylos uva ursi leaf extract, and alpha arbutin. Melanine maturation and transfer inhibitors include (but are not limited to) Pisum sativun extract, centaureidine, retinoic acid and its derivatives (retinol, retinaldehyde, retinyl palmitate, trans-retinoic acid, 13-cis retinoic acid, 9-cis retinoic acid, retinoyl glucuronoides, tretinoin, isotretinoin, etretinate, acitretine, tazarotene, adapalene, β-carotene, retinyl ester), hydroxylated diphenyl methane (US20070248633); Mixture of Water, Titanium Dioxide, Polysorbate 20, Acrylates/C10-30 Alkyl Acrylate; Crosspolymer, Polymethylmethacrylate, Trilaurin, Diacethyl Boldine (Lumisphere); Lepidum sativum sprout extract (SulforaWhite); mixture of Artocarpus heterophyllus seed extract (Whitessence); Cynara Scolymus Leaf extract (Biobenefity); Mixture of Uva-Ursi Leaf Extract, Magnesium Ascorbyl Phosphate (Melfade J); Glycyrrhiza Glabra (Licorice Eco) and soybean extract. See also The International Cosmetic Ingredient Dictionary and Handbook, 12th Ed, 2008, U.S. Personal Care Products Council's for other active agents that may inhibit tyrosinase activity, melanosome maturation and transfer, or stimulate melanine pigment degradation.

### Anti-Cellulite

Cellulite is not strictly associated to obesity since it affects not only overweight individuals but also thin individuals. The pathophysiology of cellulite is complex and involves the presence of excess subcutaneous fat, the microcirculatory system, lymphatics, inflammation, and the extracellular matrix. Cellulite is a condition of adipose tissue wherein the balance between lipolysis and lipogenesis is impaired. This imbalance is believed to have hormonal or nutritional origins. When this imbalance occurs, adipose cells grow excessively (i.e. up to 100 times their original size) by accumulating lipids. In parallel, their ability to capture sugars is amplified. The sugar excess results in a rigidifying of collagen fibers which normally provide elasticity to skin. Adipocytes saturated with lipids become trapped in this network of rigid fibers. Blood vessels become unable to properly reach inside this tissue which in turn results in water retention and inadequate toxin elimination. Over time fatty deposits pockets are generated that form characteristic dimples and bumps on the affected areas (orange-peel like appearance). The present invention also encompasses compositions comprising anti-cellulite activities.

In a specific embodiments, the present invention may contain at least one active agent that promotes fat cell metabolism through phosphodiesterase inhibiton, cyclic AMP activation, alpha-adrenergic antagonism, and/or stimulation of adiponectin production. Adiponectin sensitizes adipocytes to the action of insulin.

In a more specific embodiment, Nelumbo nucifera leaf extract plays this role in the composition of the present invention. Nelumbo nucifera leaf extract reduces fat storage in adipocytes through local increase production of adiponectin. The extract also preserves the architecture of the ECM.

Without being so limited, the following are examples of active agents that may promote anti-cellulite activity in the composition of the present invention: plant extracts (i.e. fruit, vegetable, leguminous, flower, and/or spice extracts), algae extracts, microorganisms extracts such as yeast extracts and their derivatives, ferments, proteolytic hydrolysates, peptides, animal derivative extracts and synthetic compounds. More particularly, such active agents include Mixture of Glycerin/Aqua/Coco-Glucoside/Caprylyl Glycol/Alcohol/Glaucine (Bodylift); Mixture of Hydrolyzed Celosia Cristata Flower/Seed Extract and Hydrolized Prunella Vulgaris Extract (BIOSCULPTINE); Mixture of Butylene glycol, water and nelumbo nucifera leaf extract (PRO-SVELTYL); Mixture of Water, propylene glycol and citrus aurantium amara (bitter orange) flower extract (REMODULINE); Mixture of Water, butylene glycol and Peumus boldus leaf extract (SLIMACTIVE); Cecrpia obtusa extract (SLIM FIT), theophylline, caffeine, theobromine, yohimbine, carnitine, Asiatica cantella, rutin, blend of Celosia cristata and Prunella vulgaris extracts; Nelumbo nucifera leaf extract, Cecropia obtusa extract, Peumus boldus leaf extract, citrus aurantium amara (bitter orange) flower extract, hesperetin laurate, Imperata cylindrica extract, and licorice extract.

Other active agents that have not been listed but which improve overall skin appearance and/or skin comfort and condition may be included in accordance with the present invention. Other non-limiting examples include hyaluronic acid and c-glycoside derivatives which reinforce skin barrier function (US20080226756); acyl-salicylates (preferably C3-C25 acyl salicylates) which induce heat shock response in cells thereby reducing cellular damages (WO03049692); and hydroxybenzoic acid, DEHA and DEHA salicylate for treating skin atrophy and disorders such as dandruff, acne and psoriasis (U.S. Pat. No. 6,284,750).

As used herein the terms “emulsifying agent” is meant to refer to ingredients capable of preventing the separation of immiscible substances in an emulsion, of helping to distribute evenly one substance in another, of improving texture, homogeneity, consistency and stability. In particular, they are meant to refer to at least one agent that breaks down oil into the water phase of a composition. Without being so limited, emulsifying agents that may be used in the compositions of the present invention include any emulsifying agent or combination of emulsifying agents having high resulting hydrophilic-lipophilic balance (HLB) of more than 11. In a more specific embodiment, the HLB is of between about 11 and about 16, in another more specific embodiment between about 12 and about 16, in another more specific embodiment between about 13 and about 16, in another more specific embodiment between about 14 and about 16, in another more specific embodiment between about 15 and about 16, and in another more specific embodiment is about 15.6. Such emulsifying agents or combinations of emulsifying agents are polyethylene sorbitan esters such as, but not limited to polysorbate 40; propylene glycol esters; glycerol ethylene glycol; polyethylene esters such as, but not limited to a combination of Laureth 12 (Polyethylene 600 glycol lauryl ethers) and laureth 23; PEG dilaurate-polyethylene glycol esters, cetearyl alcohol, glyceryl stearate, alkyl acrylate crosspolymer, stearic acid, emulsifying wax, sorbitan oleate, sorbitan stearate, polyethylene copolymer, sorbitan monopalmitate, polyoxyethylene sorbitan monoleate, polysorbate, polyethylene glycopolysorbate, triethanolamine, cyclopentasiloxane, dimethicone copolyol, PEG-20 stearate, PEG-23 stearate, PEG-30 dipolyhydroxystearate, sucrose distearate, PEG-100 stearate, sodium dioctylsulfosuccinate, polyacrylamide, isoparaffin, laureth-7, cetyl phosphate, DEA cetyl phosphate, glycol stearate, stearyl alcohol, cetyl alcohol, behentrimonium methosulfate and ceteareth-2, and combination thereof. The amount of emulsifying agent that may be used in accordance with the present invention is preferably less than about 2%, including but not limited to: less then about 1.95%, less then about 1.90%, less then about 1.85%, less then about 1.80%, less then about 1.75%, less then about 1.70%, less then about 1.65%, less then about 1.60%, less then about 1.55%, less then about 1.50%, less then about 1.45%, less then about 1.40%, less then about 1.35%, less then about 1.30%, less then about 1.25%, less then about 1.20%, less then about 1.15%, less then about 1.10%, less then about 1.05%, less then about 1%, less then about 0.9%, less ten about 0.8%, less then about 0.7%, less then about 0.6%, less then about 0.5%, less then about 0.4%, less then about 0.3%, less then about 0.2%, less then about 0.1% or less then about 0.05%.

In traditional compositions, once the formulation has absorbed the emulsifying agent concentration that is necessary to maintain the emulsion (break down the oil in the emulsion) some unused/free emulsifying agent may remain in the composition. This unused/free emulsifying agent may break down the lipid bilayers of the skin which might allow irritating materials to enter the skin barrier. Traditional topical compositions typically use from 2 to 7% w/w emulsifying agents. In specific embodiments, the present invention thus avoids the use of emulsifying agents altogether or reduces it to a minimum so as to prevent as much as possible the presence of unused/free emulsifying agent while maintaining the emulsion. Additional emulsifying agents that may be used in the present invention are listed in The International Cosmetic Ingredient Dictionary and Handbook, 12th Ed, 2008, U.S. Personal Care Products Council's. In specific embodiments, the compositions of the present invention advantageously comprise less than about 1.5% w/w emulsifying agent. In a more specific embodiment, it contains a maximum of about 1% w/w emulsifying agent. In another specific embodiment, it contains a maximum of about 0.9% w/w emulsifying agent. In another specific embodiment, it contains a maximum of about 0.8% w/w emulsifying agent. In another specific embodiment, it contains less than 0.8% w/w, less than 0.7% w/w, less than 0.6% w/w emulsifying agent, less than 0.5% w/w emulsifying agent, less than 0.4% w/w emulsifying agent, less than 0.3% w/w emulsifying agent, less than 0.2% w/w emulsifying agent, less than 0.1% w/w emulsifying agent. In another specific embodiment, it contains no emulsifying agent.

As used herein the term “carbomer” is used to refer to a carbomer per se, sodium carbomer, potassium carbomer, calcium potassium carbomer, an carbomer derivative, or a combination thereof. Carbomer is a polymer of acrylic acid cross-linked with a polyfunctional compound, hence, a poly (acrylic acid) or polyacrylate.

As used herein the terms “Acryloyldimethyltaurate derivative” are used to refer to a polymer (e.g., copolymer, crosspolymer) of the Acryloyldimethyltaurate family or a mixture of polymers from this family. In a more specific embodiment, the Acryloyldimethyltaurate derivative is an “ammonium Acryloyldimethyltaurate derivative”. Without being so limited, this family includes ammonium acryloyldimethyltaurate/vinyl pyrrolidone copolymer (e.g., Aristoflex AVC); ammonium acryloyldimethyltaurate/Beheneth-25 Methacrylate Crosspolymer (e.g., Aristoflex HMB); Caprylic/Capric Triglyceride (and) ammonium acryloyldimethyltaurate/VP Copolymer (and) Trilaureth-4 Phosphate (and) Polyglyceryl-2-Sesquiisostearate (e.g., Aristoflex AVL); Ammonium Acryloyldimethyltaurate/Steareth-25 Methacrylate Crosspolymer (e.g., Aristoflex HMS), and mixtures thereof, etc.

As used herein, the term “active agent” is meant to refer to an ingredient that has at least one anti-aging activity or at least one activity against a skin condition or disorder described herein. In a specific embodiment, the compositions of the present invention comprise about 20% to about 90% of active agents w/w of the total composition. In another specific embodiment, the compositions of the present invention comprise about 25% to about 90% of active agents w/w of the total composition. In another specific embodiment, the compositions of the present invention comprise about 30% to about 90% of active agents w/w of the total composition. In another specific embodiment, the compositions of the present invention comprise about 35% to about 90% of active agents w/w of the total composition. In another specific embodiment, the compositions of the present invention comprise about 40% to about 90% of active agents w/w of the total composition. In an other specific embodiment, the compositions of the present invention comprise about 45% to about 90% of active agents w/w of the total composition. In another specific embodiment, the compositions of the present invention comprise about 50% to about 90% of active agents w/w of the total composition. In another specific embodiment, the compositions of the present invention comprise at least about 20% (21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%) of active agents w/w of the total composition.

As used herein, the term “isolated” in the expression “isolated active agent” means altered “by the hand of man” from its natural state (i.e. if it occurs in nature, it has been changed or removed from its original environment) or it has been synthesized in a non-natural environment (e.g., artificially synthesized). These terms do not require absolute purity (such as a homogeneous preparation) but instead represents an indication that it is relatively more pure than in the natural environment. For example, an active agent naturally present in natural plant extracts (i.e. fruit, vegetable, leguminous, flower, and/or spice extracts), in algae extracts, microorganisms extracts such as yeast extracts and their derivatives, ferments, proteolytic hydrolysates, peptides or animal derivative extracts is not “isolated”, but the same active agent separated (e.g., about 90-95% pure at least) from the coexisting materials of its natural state is “isolated” as this term is employed herein. As used herein, a natural extract constitutes a single active agent.

Topical compositions of the present invention may take diverse forms such as solutions, suspensions, lotions, tinctures, gels, creams, sprays, emulsions, droplets, milks, sticks, ointments or liposomes (at least a portion of the multitarget formulation being present in liposomes) to provide a plurality of different topical formulations for the compositions of the present invention. Applications of the compositions of the present invention include topically applicable cosmetic compositions. Non-limitative examples of such topically applicable compositions include skin care cream, cleansing cream, skin care lotion, skin care gel, skin care foam, sun care composition, make-up removal cream, make-up removal lotion, foundation cream, liquid foundation, bath and shower preparation, deodorant composition, antiperspirant composition, shaving products composition, after-shave gel or lotion, beauty aids composition, depilatory cream, soap composition, hand cleaner composition, cleansing bar, baby care, hair care, shampoo, setting lotion, treatment lotion, hair cream, hair gel, coloring composition, restructuring composition, permanent composition, anti-hair loss composition, or any other composition which is adapted for the use in a topical cosmetic regimen.

Without being so limited, the compositions of the present invention may include, in addition to anti-aging agents targeting one or more of mechanisms described above, other types of agents possessing activities beneficial to the skin. Hence without being so limited, the compositions of the present invention may comprise anaesthesic agent, anti-acne agent, anti-aging agent, antibacterial agent, anticellulite agent, antifungal agent, anti-inflammatory agent, anti-irritation agent, anti-oxidant agent, antiparasitic agent, antipollution agent, antipruritic agent, anti-rosacea agent, anti-seborrhea agent, anti-stress agent, anti-telangiectasia agent, antiviral agent, anti-wrinkle agent, baby care agent, bath and body agent, calming agent, cleansing agent, collagen synthesis agent, DNA protection and repair agent, elastase inhibitory agent, exfoliant agent, facial peeling agent, firming agent, foot care agent, free radical scavenging agent, glycosaminoglycan synthesis agent, anti-glycation agent, immune function modulator agent, keratolytic agent, lift agent, make-up remover agent, melanogenesis stimulator agent, matrix metalloproteinase inhibitory agent, moisturizing agent, oil absorbing agent, osmoregulator agent, anti-photoaging agent, protecting agent, protein repair agent, proteasome stimulator agent, rejuvenating agent, regenerating agent, restructuring agent, sensitive skin agent, shaving product agent, sirtuin stimulator agent, skin defense enhancer agent, skin lightening agent, skin clarifier agent, skin repair agent, slimming agent, smoothing agent, softening agent, soothing agent, sun care agent, sunless tanning agent, tensing agents and whitening agent, or any other agent adapted for use in a cosmetic regimen that comprises topical application of said cosmetic composition.

The topical compositions of the present invention may further comprise additional excipients such as buffering agent, carrier agent, chelating agent, conditioning agent, coloring agent, detackifying agent, emollient agent, film-forming agent, foaming agent, humectant agent, lactylate agent, lipophilic agent, neutralizing agent, oil agent, opacifier agent, preservative agent, solubilizing agent, solvent agent, stabilizing agent, emulsifying agent, thickening agent, viscosity increasing agent, water absorbing agent, light diffracting agent and wetting agent. See also The International Cosmetic Ingredient Dictionary and Handbook, 12th Ed, 2008, U.S. Personal Care Products Council's for other excipients.

The terms “buffering agents” or “buffer” refer to salts of bases/acids, compatible with the nature of the skin and with its pH. Sodium acetate is an example of a frequently used buffering agent. The pH of the compositions of the present invention as desirably as close as possible to the pH of the skin. Without being so limited, the compositions of the present invention have a pH ranging from about 4.5 to about 6.5, in a more specific embodiment between from about 4.75 to about 5.25, and in another more specific embodiment from about 5.3 to about 6.5.

The terms “carrier agents” as used herein are meant to refer to ingredients capable of aiding the application of the active agent. Isohexadecane is an example of a frequently used carrier.

The terms “chelating agents” as used herein are meant to refer to ingredients capable of binding mono and divalent cations. Maximize efficacy and longevity of the material. Without being so limited, it includes at least one of tetrasodium EDTA and disodium EDTA, EDTA and trisodium EDTA, and combinations thereof.

The terms “conditioning agents” as used herein are meant to refer to ingredients with lubricating action and hydrating effect. Without being so limited, it includes at least one of cetrimonium chloride, dicetyldimonium chloride, trideceth-12, quaternium-Z7, quaternium-18, polyquaternium-10, behentrimonium methosulfate, cetearyl alcohol, stearamidopropyl dimethylamine, trimethylsilylamodimethicone, isolaureth-6, octoxynol-4, dimethicone, dimethiconol, cyclopentasiloxane, pareth-7, pareth-9, linoleic acid, glycerin and combinations thereof.

The term “solvent” as used herein is meant to refer to non-aqueous or aqueous ingredients able to solubilize other agents. Without being so limited, aqueous solvents include water. Without being so limited non-aqueous solvents include at least one of propylene glycol, polyethylene glycol, glycerol, dimethylacetamide, N-methylpyrrolidone and combinations thereof.

The terms “detackifying agents” and “lubricating agents” are used interchangeably and are meant to refer herein to ingredients capable of adsorbing onto tacky materials and reduce their tendency to adhere or are capable of adding slipperiness and of reducing friction to improve application on the skin. Without being so limited, it includes at least one of cyclopentasiloxane, dimethicone, dimethicone copolyol and vinyl dimethicone, phenyl trimethicone, isopropyl esters, isostearate esters, dimethyl sebacate and dipropyl sebacate; mixture of phenyl trimethicone and polysilicone 11; mixture of dimethicone and polysilicone-11; mixture of cyclomethicone and polysilicone-11; mixture of cyclomethicone/cyclopentasiloxane; and HDI/Trimethylol Hexyllactone Crosspolymer and combinations thereof.

The term “fragrance” as used herein is meant to refer to an ingredient that produces or masks an odour. Fragrances appropriate for use in topical compositions are well known in the art and include artificial or natural fragrances such as essential oils.

The terms “anticaking agent” as used herein are meant to refer to ingredients capable of adsorbing excess moisture or of coating particles and making them water repellent. This term is usually reserved for solid product. Some anticaking agents are soluble in water; others are soluble in alcohols or other organic solvents. Without being so limited, it includes at least one of HDI/Trimethylol Hexyllactone Crosspolymer, aluminum starch octenylsuccinate and combinations thereof.

The terms “emollient agents” as used herein are meant to refer to ingredients with lubricating action and hydrating effect. Without being so limited, it includes at least one of isopropyl palmitate, sunflower seed oil, mineral oil, stearyl stearate, isopropyl myristate, lanolin, caprylic, capric triglyceride, cyclopentasiloxane, dimethicone, vinyl dimethicone, bis-phenylpropyl dimethicone, alkyl dimethicone, sorbitan stearate, sucrose distearate, myristyl alcohol, myristyl lactate, cetyl acetate, dicaprylyl ether, floraester-20, maleated soybean oil, cyclomethicone, squalane, shea butter, hydrogenated coconut oil, isopropyl palmitate, diisostearoyl trimethylolpropane siloxy silicate and alkyl benzoate and combinations thereof.

The terms “film forming agents” as used herein are meant to refer to ingredients capable of forming a dimensionally stable and continuous film to minimize the formula tackiness. Without being so limited, it includes at least one of wheat protein, eicosene copolymer, perfluoromethylisopropyl ether, PVP (polyvinylpirrolidone), diisostearoyl trimethylolpropane siloxy silicate, trimethylsiloxysilicate, dimethicone, vinyl dimethicone and cyclopentasiloxane and combinations thereof.

The terms “foaming agents” as used herein are meant to refer to ingredients capable of regulating the amount of air in a product. Without being so limited, it includes at least one of lauramide DEA and cocamide MEA, disodium laureth sulfosuccinate, disodium N-octadecyl sulfosuccinamate, ammonium lauryl sulphate, triethanolamine lauryl sulfate, sodium lauryl sulphate, sodium 2-ethylhexylsulfate and combinations thereof.

The terms “humectant agents” as used herein are meant to refer to ingredients capable of maintaining constant humidity and retaining moisture. Without being so limited, it includes at least one of glycerine, PEG-8, butylene glycol, propylene glycol and combinations thereof.

The terms “neutralizing agents” as used herein are meant to refer to ingredients capable of changing the acid-alkaline balance. Without being so limited, it includes at least one of triethanolamine, sodium hydroxide and combinations thereof.

The terms “opacifier agents” as used herein are meant to refer to ingredients capable of changing the look of a clear or translucent product to a creamier or pearlier one. Without being so limited, it includes at least one of glyceryl stearate, PEG-100 stearate and combinations thereof.

The terms “preservative” or “preservative agent” as used herein are meant to refer to any ingredient capable of retarding or preventing microbial (gram negative and/or positive) yeast, fungi, or chemical spoilage, and protecting against discoloration and loss of activity. Without being so limited, it includes at least one of DMDM hydantoin, methylparaben, propylparaben, phenoxyethanol, ethylparaben, butylparaben, imidazolidinyl urea, diazolidinyl urea, symdiol-68, symdiol-68T, cosmocil-CQ, quaternium-8, quaternium-14, quaternium-15, propylene glycol, dehydroacetic acid and its derivatives, methylchloroisothiazolinone, methylisothiazolinone, 1,2-hexanediol, germaben and combinations thereof. A combination of preservatives could be useful to protect again gram positive bacteria, gram negative bacteria, fungi, and/or yeast.

The terms “solubilizing agents” as used herein are meant to refer to ingredients capable of allowing incompatible ingredients to become part of a homogeneous solution. Without being so limited, it includes at least one of polysorbate, ceteareth, steareth, PEG and combinations thereof.

The terms “stabilizing agents” as used herein are meant to refer to ingredients capable of maintaining physical and chemical properties during and after processing, preventing or limiting changes in the physical properties of a substance during product life. Without being so limited, it includes at least one of polyethylene, sodium chloride, stearyl alcohol, xanthan gum, tetrasodium EDTA, carbopol and its derivatives, dimethicone copolyol, and combinations thereof.

The term “sunscreen” as used herein is meant to refer to ingredients that protect skin from the effects of the sun's rays. Sunscreens act by absorbing ultraviolet radiation or by reflecting the incident light. Without being so limited, it includes at least one chemical sunscreen such as octinoxate (UVB), benzophenone-3 (UVB), Octyl salicylate (UVB), Octyl methoxycinnamate, Octisalate, Octicrylene, Parsol 1789 (avobenzone) (UVA) and/or at least one physical sunscreen such as zinc oxide (UVB)), and titanium dioxide (UVA), and combinations thereof.

As used herein, the terms “thickening agents” are meant to refer to ingredients capable of absorbing water to impart body and/or improve the consistency or texture, increase the viscosity of the external phase of the emulsion and/or stabilize an emulsion. Without being so limited, it includes at least one of stearic acid, magnesium aluminum silicate, carbomer, alkyl acrylate crosspolymer, polyacrylamide, isoparaffin, laureth-7, cetyl alcohol, xanthan gum, alkyl dimethicone, hydroxyethylcellulose, glyceryl stearate, pentaerythrityl tetrastearate, stearyl alcohol and polyquaternium-10, glycerol, poly(ethylene glycol) (PEG), propylene glycol, polyvinylpyrolidone, dextran and combinations thereof.

As used herein, the terms “viscosity increasing agent” are meant to refer to ingredients capable of controlling the degree of fluidity and the internal resistance to flow exhibited by a fluid. Without being so limited, viscosity-increasing agent that may be used in the compositions of the present invention include at least one of sodium carbomer, acryloyldimethyltaurate/vinyl pyrrolidone copolymer, magnesium aluminum silicate, caprylyl glycol, myristyl alcohol, Nylon-12 and combinations thereof. In specific embodiments, compositions of the present invention typically have a viscosity ranging between about 8,000 to about 50,000.

As used herein, the terms “water absorbing” agents are ingredients capable of absorbing the product's water to maintain the moisture. Without being so limited, it includes at least one of carboxyvinyl polymer, acrylic copolymer, polyacrylamide, polysaccharides, natural gum, clay, modified clay, metallic salt, fatty acid and combinations thereof.

As used herein, the terms “light-diffracting agent” are meant to refer to ingredients capable of emitting or diffusing visible light and therefore reduce the appearance of wrinkles. Without being so limited, they include nylon-12.

As used herein, the terms “wetting agents” are meant to refer to ingredients capable of reducing the surface tension of the water for better penetration or spread over the surface. Without being so limited, it includes at least one of caprylate, caprylyl glycol, glyceryl caprate, polyglyceryl-2 caprate, polyglyceryl-6, polyglyceryl-3 laurate and TEA-laureth sulfate and combinations thereof.

As used herein the terms “colouring agent” are meant to refer to ingredients capable of colouring compositions. Without being so limited, such agent may be pigments such as but not limited to at least one member of the Mica series, coloured titanium dioxide, iron oxide and combinations thereof.

As used herein the terms “anti-browning agent” are meant to refer to ingredients capable of preventing browning of a formulation. Without being so limited it includes at least one of sodium metabisulfite, disodium pyrosulfite, disodium disulfite, sodium pyrosulfite and combinations thereof.

As used herein the terms “skin aging sign” is meant to refer to fine lines, wrinkles, inflammation, redness, telangiectasia, skin sagging, excess sebum, enlarged pores, dark circles, loss of skin firmness, brown spot, increased skin thickness, sun damage, hyper pigmented skin, dull skin, loss of skin elasticity and collagen content, bags under eyes, lentigines, melasmas, disturbance of sebum production, dehydration, loss of skin comfort, and skin devitalization (reduced metabolic activity).

As used herein the terms “skin condition or disorder” is meant to refer to rosacea, acne-rosacea, spider veins, skin flushing, acne, pimples, dermatitis, rashes, irregular skin tone, cellulite, clogged pores, and blotches.

As used herein, the term “reducing” in the expression “reducing skin aging sign” or “reducing skin condition or disorder” is meant to refer to a reduction of a pre-existing aging skin sign, or skin condition or disorder, respectively. It encompasses complete or partial correction/treatment of the aging sign or skin condition or disorder, respectively. As used herein, the term “preventing” in the expression “preventing skin aging sign” or “preventing skin condition or disorder” is meant to refer to a delay in the initiation of, or a complete or partial prevention of a skin aging sign, or skin condition or disorder, respectively.

As used herein the terms “effective amount” as it relates to a composition of the present invention is an amount that effectively prevents or reduces a skin aging sign or a skin condition or disorder of the subject. It typically constitutes an amount sufficient to cover the skin that is to be treated and the application rate is typically once or twice a day. The effective amount may vary depending on the form of the composition (e.g., gel, cream, serum, etc.) and the type of skin of the subject.

The compositions of the present invention may be packaged in any suitable manner, including but not limited to, a jar, a bottle, a tube, a stick, a roller-ball applicator, an aerosol spray device, etc., in the conventional manner. For instance, the active agents and the topically-acceptable vehicle may be provided in larger quantities from which the needed amount could be withdrawn using various measuring devices, such as a measuring spoon or cup for solids, or a calibrated vial or dropper for liquids. The compositions of the present invention may be spread onto a substrate and then subsequently packaged. Suitable substrates include dressings, including film dressings, and bandages. In an embodiment, the kit or package may comprise instructions for use/application, e.g., instructions for preventing or reducing a skin condition or a skin aging sign.

As used herein, the term “about” when used in relation to ranges apply to both ends of the range. It is used to reflect the relative precision of the equipment and process used to obtain and to characterize the compositions of the present invention.

The articles “a,” “an” and “the” are used herein to refer to one or to more than one (i.e., to at least one) of the grammatical object of the article.

The term “including” and “comprising” are used herein to mean, and re used interchangeably with, the phrases “including but not limited to” and “comprising but not limited to”.

The term “such as” is used herein to mean, and is used interchangeably with, the phrase “such as but not limited to”.

As used herein the term “subject” is meant to refer to any mammal including human, mice, rat, dog, cat, pig, cow, monkey, horse, etc. In a particular embodiment, it refers to a human.

The present invention is illustrated in further details by the following non-limiting examples.

### Example 1

Topical multi-target anti-age formulation

| Ingredient (trademark) | Function(s) | Group # | % W/w |
|---|---|---|---|
| Water | Moisturizer | 1 | 65.05 |
| Methylparaben | Preservative | 1 | 0.25 |
| Disodium EDTA (dissolvine Na2) | Chelating agent | 1 | 0.1 |
| Sodium carbomer (PNC-430) | Emulsion stabilizing agent, viscosity | 2 | 1 |
| | increasing agent | | |
| Ammonium Acryloyldimethyltaurate/VP copolymer | Viscosity increasing agent | 3 | 0.5 |
| (Aristoflex AVC) | | | |
| Octinoxate | Active sunscreen | 4 | 4 |
| Dimethicone, polysilicone-11 (Gransil DMG-6) | Detackifying agent | 4 | 4.5 |
| Phenyl trimethicone, polysilicone 11 (Gransil PM | Detackifying agent | 4 | 3.75 |
| gel) | | | |
| Squalane HQ (olive oil derived) | Hydrating agent, emollient, anti-oxidant | 4 | 1 |
| Tocopheryl Acetate (Vitamin E acetate) | Hydrating agent | 4 | 0.1 |
| Aluminum Benzoate (cosmetic astringent), mica, | Surface modifier, UV light absorber, | 4 | 0.07 |
| Disodium Distyrybiphenyl Disulfonate (surface | cosmetic astringent | | |
| modifier, UV light absorber), Aluminum Chloride | | | |
| (Covazur Z03) | | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer (BPD- | Detackifying agent, humectant agents | 5 | 1 |
| 500/plastic powder D) | | | |
| Cyclomethicone, polysilicone-11 (Gransil GCM) | Detackifying agent | 6 | 4.5 |
| Cyclomethicone/cyclopentasiloxane (Dow corning | Detackifying agent | 7 | 2 |
| 345 fluid) | | | |
| Glycosaminoglycans (MRTEX complex) | Anti-mmps; skin matrix integrity, anti- | 7 | 3 |
| | inflammatory | | |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese chloride | Anti-oxidant, oxygenation, DNA self-repair | 7 | 0.03 |
| (Euk-134) | | | |
| Fragrance (Juniper Breeze) | Produces or masks odours | 7 | 0.15 |
| Glycerin, butylene glycol, water, carbomer (emulsion | Collagen synthesis | 7 | 3 |
| stabilizing agent), polysorbate-20, palmitoyl | | | |
| pentapeptide-4 (Matrixyl 3000) | | | |
| Propylene Glycol (humectant, solvent), Commiphora | Hydrating agent | 7 | 0.1 |
| Myrrha Extract, Equisetum Arvense Extract, Triticum | | | |
| Vulgare Germ extract, Humulus lupulus Extract | | | |
| (330-Regederme HS) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

1. In a stainless steel, jacketed kettle equipment with variable, high speed propeller agitation (2,400 RPMs) or turbine mixer and sweep agitation, ingredients of group 1 (water and preservatives) as identified in the Table above were combined under adequate shear heated to 80-82 C. Top and bottom of tank were observed to insure solubility of methylparaben prior to proceeding. Mixture was then cooled to 76-78° C.

2. Ingredients of group 2 were then added to the mixture to transform the mixture into a gel. Agitation was increased up to 2,400 RPMs when useful.

3. Ingredients of group 3 were then added. Agitation was increased up to 2,400 RPMs when useful.

4. In a separate kettle with a propeller type agitation. the ingredients of group 4 were combined under agitation and heated to 80-82° C.

5. At 80-82 C., ingredients of group 4 were then slowly added to the mixture of ingredients of group 1, 2 and 3, under adequate shear agitation. Adjustments were made when useful in cases where batch became heavy after the addition of the gums (ingredients of groups 2 and 3).

6. Temperature and agitation were maintained until gel was well formed and uniform. The mixture was then cooled to 70° C. At that temperature, the ingredients of group 5 were slowly sifted (plastic powder) into the batch. For instance, when the batch appeared too liquid, a further amount of Aristoflex™ AVC was added after the emulsion was formed with ingredients of groups 1, 2 and 4 at approximately 70 degrees centigrade to increase the viscosity of the batch.

7. The mixture was then cooled to 50° C. At that temperature, the ingredients of group 6 (Gel) were added to the batch. When useful, sweep agitation was used after incorporation of ingredients of that group. The mixture was then cooled to 35-40° C.

8. At 35-40° C. combined premixed ingredients of group 7 were added each by each to the batch very slowly, while adjusting energy if necessary.

9. The mixture was cooled to 25° C. and mixing was continued until the mixture was uniform.

### Specifications

The pH of the composition was of 6.26+/−0.25 with a range of 6.01-6.51. The viscosity calculated on a Brookfield LVT Model DV I at 25 degrees Centigrade/1 minute, spindle #4 at 3 RPMs, dial reading at 64% was of 113,000 cps with a range of between about 100,000 to 125,000 cps. The color was off white to slightly beige. The odor was characteristic to slightly fruity. The appearance was that of a moderately viscous cream with a smooth texture. The specific gravity (density) was of 1.008+/−0.02 on a stainless steel grease pychnometer at 25 degrees centigrade with a range of 0.988-1.028.

### Example 2

Topical multi-target anti-age formulation with added retinol and creatine

| Ingredient (trademark) | Function(s) | Group # | % W/w |
|---|---|---|---|
| Water | Moisturizer | 1 | 65.21 |
| Methylparaben | Preservative | 1 | 0.25 |
| Disodium EDTA (dissolvine Na2) | Chelating agent | 1 | 0.1 |
| Sodium carbomer (PNC-430) | Emulsion stabilizing agent, viscosity | 2 | 1 |
| | increasing agent | | |
| Ammonium Acryloyldimethyltaurate/VP copolymer | Viscosity increasing agent | 3 | 0.5 |
| (Aristoflex AVC) | | | |
| Octinoxate | Active sunscreen | 4 | 4 |
| Dimethicone, polysilicone-11 (gransil DMG-6) | Skin conditioning, skin protectant | 4 | 4.4 |
| Phenyl trimethicone, polysilicone 11 (Gransil PM gel) | Detackifying agent | 4 | 3.65 |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium Distyrybiphenyl | Surface modifier, UV light absorber, | 4 | 0.1 |
| Disulfonate, Aluminum Chloride (Covazur Z03) | cosmetic astringent | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer (BPD- | Hydrating agent | 5 | 1 |
| 500/plastic powder D) | | | |
| Cyclomethicone, polysilicone-11 (Gransil GCM) | Detackifying agent | 6 | 4.4 |
| Cyclomethicone/cyclopentasiloxane (Dow corning | Detackifying agent | 7 | 2 |
| 345 fluid) | | | |
| Glycosaminoglycans (MRTEX complex) | Anti-mmps; skin matrix integrity, anti- | 7 | 3 |
| | inflammatory | | |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese chloride | Anti-oxidant, oxygenation, DNA self-repair | 7 | 0.03 |
| (Euk-134) | | | |
| Retinol (retinol 50c liquid + polysorbate 20) | Cellular renewal | 7 | 0.1 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer (emulsion | Collagen synthesis | 7 | 3 |
| stabilizing agent), polysorbate-20, palmitoyl | | | |
| pentapeptide-4 (Matrixyl 3000) | | | |
| Propylene Glycol (humectant, solvent), Commiphora | Hydrating agent | 7 | 0.1 |
| Myrrha Extract, Equisetum Arvense Extract, Triticum | | | |
| Vulgare Germ extract, Humulus lupulus Extract | | | |
| (330-Regederme HS) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

The composition was prepared by following the steps described in Example 1 above.

### Specifications

The pH of the composition was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Accelerated Stability Testing

The stability of the product was tested with an accelerated aging process. It was subjected to 4° C., 25° C. and 45° C. during 3 months. Measures and observations were taken at time 0, 1 month, 2 months and 3 months. The following parameters were measures: pH, viscosity, colour, organoleptic observations, organoleptic perception on the skin and appearance and signs of separation. Observations of the appearance were made following sharp variations of temperature and after freeze-thaw cycles.

The pH remained stable at all temperatures. Viscosity varied but no liquefaction or excessive thickening was observed. The fragrance was stable although its intensity progressively weakened over time. The fragrance started to change at 45° C. at 3 months, which could be equated, to about 2 years at 25° C. The colour was unstable. It darkened and the browning rate correlated to the temperature. The cream darkened by 2 No Pantone within two weeks and then gained 1 No Pantone per month at 45° C. Light had little effect on browning rate. It was estimated that the cream would become dark brown within two years %. The appearance of the cream also changed. A syneresis was observed at 25° C., and it increased with temperature changes: freeze-thaw cycles and heat. After three months at 45° C., syneresis was observed with a loss of homogeneity.

### Clinical Trial

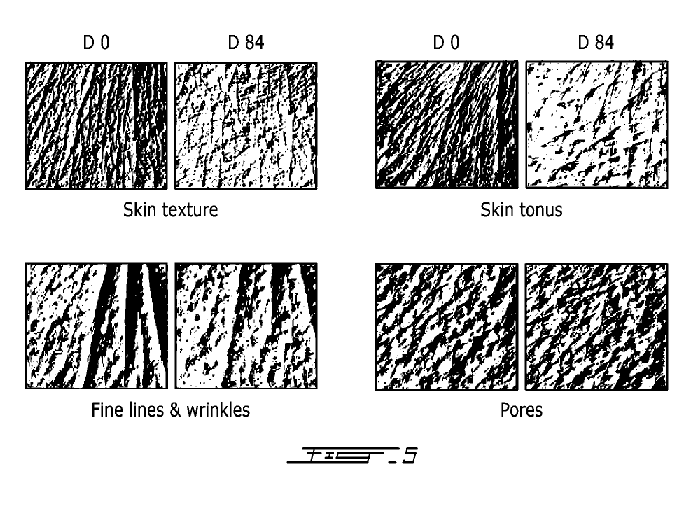

The efficacy of the composition described in Example 2 was assessed in a monocentric open-ended study conducted on 20 healthy female volunteers aged 35 to 62. The treatment involved twice daily applications of the serum to the eye-contour, face and neck area for a period of twelve weeks. A hydrating gel was combined with the serum starting on the third week in the study. Measurements of skin hydration with Corneometer™; wrinkle profile by the aid of silicone imprints from the eye contour zone, and digital photographs were obtained for each volunteer before (D=0) and after treatment (D=7, D=28, D=56 and D=84). The results indicated that the serum has anti-age properties, it improved the wrinkle parameters, the tone, texture, uniformity, thickness and general appearance of the skin and this despite a dehydrating effect, which may have overshadowed the true anti-wrinkle and anti-aging potential benefits of the cream. It was found for instance that the mean number of the wrinkles reduced from 97 to 91 after 84 days of treatment with the test serum. The total surface area for the wrinkles also reduced from 17.97 to 15.26 and the total length reduced from 69.15 to 59.38. See FIG. 5 for an example of results from this study.

### Example 3

Topical multi-target anti-age formulation with reduced retinol concentration to reduce irritation

| Ingredient (trademark) | Function(s) | Group # | % W/w |
|---|---|---|---|
| Water | Moisturizer | 1 | 65.26 |
| Methylparaben | Preservative | 1 | 0.25 |
| Disodium EDTA (dissolvine Na2) | Chelating agent | 1 | 0.1 |
| Sodium carbomer (PNC-430) | Emulsion stabilizing agent, viscosity | 2 | 1 |
| | increasing agent | | |
| Ammonium Acryloyldimethyltaurate/VP copolymer | Viscosity increasing agent | 3 | 0.5 |
| (Aristoflex AVC) | | | |
| Octinoxate | Active sunscreen | 4 | 4 |
| Dimethicone, polysilicone-11 (gransil DMG-6) | Skin conditioning, skin protectant | 4 | 4.4 |
| Phenyl trimethicone, polysilicone 11 (Gransil PM gel) | Detackifying agent | 4 | 3.65 |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium Distyrybiphenyl | Surface modifier, UV light absorber, | 4 | 0.1 |
| Disulfonate, Aluminum Chloride (Covazur Z03) | cosmetic astringent | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer (BPD- | Detackifying agent | 5 | 1 |
| 500/plastic powder D) | | | |
| Cyclomethicone, polysilicone-11 (Gransil GCM) | Detackifying agent | 6 | 4.4 |
| Cyclomethicone/cyclopentasiloxane (Dow corning | Skin conditioning, Detackifying agent | 7 | 2 |
| 345 fluid) | | | |
| Glycosaminoglycans (MRTEX complex) | Anti-mmps; skin matrix integrity, anti- | 7 | 3 |
| | inflammatory | | |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese chloride | Anti-oxidant, oxygenation, DNA self-repair | 7 | 0.03 |
| Euk-134) | | | |
| Retinol (retinol 50c liquid + polysorbate 20) | Cellular renewal | 7 | 0.05 |
| creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer (emulsion | Collagen synthesis | 7 | 3 |
| stabilizing agent), polysorbate-20, palmitoyl | | | |
| pentapeptide-4 (Matrixyl 3000) | | | |
| Propylene Glycol (humectant, solvent), Commiphora | Hydrating agent | 7 | 0.1 |
| Myrrha Extract, Equisetum Arvense Extract, Triticum | | | |
| Vulgare Germ extract, Humulus lupulus Extract | | | |
| (330-Regederme HS) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Example 4

Topical multi-target anti-age formulation with added controlled delivery
system and reduced retinol concentration to reduce irritation

| Ingredient (trademark) | Function(s) | Group # | % W/w |
|---|---|---|---|
| Water | Moisturizer | 1 | 63.26 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer-15) | Controlled delivery system | 1 | 2 |
| Sodium carbomer (PNC-430) | Emulsion stabilizing agent, viscosity | 2 | 1 |
| | increasing agent | | |
| Ammonium Acryloyldimethyltaurate/VP copolymer | Viscosity increasing agent | 3 | 0.5 |
| (Aristoflex AVC) | | | |
| Octinoxate | Active sunscreen | 4 | 4 |
| Dimethicone, polysilicone-11 (gransil DMG-6) | Skin conditioning, skin protectant | 4 | 4.4 |
| Phenyl trimethicone, polysilicone 11 (Gransil PM gel) | Detackifying agent | 4 | 3.65 |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium Distyrybiphenyl | surface modifier, UV light absorber, | 4 | 0.1 |
| Disulfonate, Aluminum Chloride (Covazur Z03) | cosmetic astringent | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer (BPD- | Detackifying agent | 5 | 1 |
| 500/plastic powder D) | | | |
| Cyclomethicone, polysilicone-11 (Gransil GCM) | Detackifying agent | 6 | 4.4 |
| Cyclomethicone/cyclopentasiloxane (Dow corning | Detackifying agent | 7 | 2 |
| 345 fluid) | | | |
| Glycosaminoglycans (MRTEX complex) | Anti-mmps; skin matrix integrity, anti- | 7 | 3 |
| | inflammatory | | |
| Hyaluronic Acid, water (hyasol BT 1%) | Hydrating agent | 7 | 2 |
| Epilobium Angustifolium extract | anti-irritation agent | 7 | 2 |
| Ethylbisiminomethylguaiacol manganese chloride | Anti-oxidant, oxygenation, DNA self-repair | 7 | 0.03 |
| (Euk-134) | | | |
| Retinol (retinol 50c liquid + polysorbate 20) | Cellular renewal | 7 | 0.05 |
| Creatine (TegoCosmo c-100) | ATP stimulation | 7 | 0.01 |
| Fragrance (Juniper Breeze) | Produce or masks odours | 7 | 0.15 |
| Glycerin, butylene glycol, water, carbomer (emulsion | Collagen synthesis | 7 | 3 |
| stabilizing agent), polysorbate-20, palmitoyl | | | |
| pentapeptide-4 (Matrixyl 3000) | | | |
| Propylene Glycol (humectant, solvent), Commiphora | Hydrating agent | 7 | 0.1 |
| Myrrha Extract, Equisetum Arvense Extract, Triticum | | | |
| Vulgare Germ extract, Humulus lupulus Extract | | | |
| (330-Regederme HS) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Example 5

Topical multi-target anti-age formulation with reduced Ethylbisiminomethylguaiacol
manganese chloride to obtain a whiter composition

| Ingredient (trademark) | Function(s) | Group # | % W/w |
|---|---|---|---|
| Water | Moisturizer | 1 | 65.225 |
| Methylparaben | Preservative | 1 | 0.25 |
| Disodium EDTA (dissolvine Na2) | Chelating agent | 1 | 0.1 |
| Sodium carbomer (PNC-430) | Emulsion stabilizing agent, viscosity | 2 | 1 |
| | increasing agent | | |
| Ammonium Acryloyldimethyltaurate/VP copolymer | Viscosity increasing agent | 3 | 0.5 |
| (Aristoflex AVC) | | | |
| Octinoxate | Active sunscreen | 4 | 4 |
| Dimethicone, polysilicone-11 (gransil DMG-6) | Skin conditioning, skin protectant | 4 | 4.4 |
| Phenyl trimethicone, polysilicone 11 (Gransil PM | Detackifying agent | 4 | 3.65 |
| gel) | | | |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium | Surface modifier, UV light absorber, | 4 | 0.1 |
| Distyrybiphenyl Disulfonate, Aluminum Chloride | cosmetic astringent | | |
| (Covazur Z03) | | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer (BPD- | Detackifying agent | 5 | 1 |
| 500/plastic powder D) | | | |
| Cyclomethicone, polysilicone-11 (Gransil GCM) | Detackifying agent | 6 | 4.4 |
| Cyclomethicone/cyclopentasiloxane (Dow corning | Skin conditioning, Detackifying agent | 7 | 2 |
| 345 fluid) | | | |
| Glycosaminoglycans (MRTEX complex) | Anti-mmps; skin matrix integrity, anti- | 7 | 3 |
| | inflammatory | | |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese chloride | Anti-oxidant, oxygenation, DNA self-repair | 7 | 0.015 |
| (Euk-134) | | | |
| Retinol (retinol 50c liquid + polysorbate 20) | Cellular renewal | 7 | 0.1 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer (emulsion | Collagen synthesis | 7 | 3 |
| stabilizing agent), polysorbate-20, palmitoyl | | | |
| pentapeptide-4 (Matrixyl 3000) | | | |
| Propylene Glycol (humectant, solvent), Commiphora | Hydrating agent | 7 | 0.1 |
| Myrrha Extract, Equisetum Arvense Extract, Triticum | | | |
| Vulgare Germ extract, Humulus lupulus Extract | | | |
| (330-Regederme HS) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Example 6

Topical multi-target anti-age formulation with added controlled delivery system
reduced Ethylbisiminomethylguaiacol manganese chloride to reduce irritation

| Ingredient (trademark) | Function(s) | Group # | % W/w |
|---|---|---|---|
| Water | Moisturizer | 1 | 63.225 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Delivery system to avoid irritation and inflammation | 1 | 2 |
| 15) | | | |
| Sodium carbomer (PNC-430) | Emulsion stabilizing agent, viscosity increasing | 2 | 1 |
| | agent | | |
| Ammonium Acryloyldimethyltaurate/VP | Viscosity increasing agent | 3 | 0.5 |
| copolymer (Aristoflex AVC) | | | |
| Octinoxate | Active sunscreen | 4 | 4 |
| Dimethicone, polysilicone-11 (gransil DMG- | Skin conditioning, skin protectant | 4 | 4.4 |
| 6) | | | |
| Phenyl trimethicone, polysilicone 11 | Detackifying agent | 4 | 3.65 |
| (Gransil PM gel) | | | |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer | Detackifying agent | 5 | 1 |
| (BPD-500/plastic powder D) | | | |
| Cyclomethicone, polysilicone-11 (Gransil | Detackifying agent | 6 | 4.4 |
| GCM) | | | |
| Cyclomethicone/cyclopentasiloxane (Dow | Skin conditioning, Detackifying agent | 7 | 2 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Epilobium Angustifolium extract | anti-irritation agent | 7 | 2 |
| Ethylbisiminomethylguaiacol manganese | Anti-oxidant, oxygenation, DNA self-repair | 7 | 0.015 |
| chloride (Euk-134) | | | |
| Retinol (retinol 50c liquid + polysorbate 20) | Cellular renewal | 7 | 0.1 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Example 7

Topical multi-target anti-age formulation with reduced retinol to reduce irritation

| Ingredient (trademark) | Function(s) | Group # | % W/w |
|---|---|---|---|
| Water | Moisturizer | 1 | 65.285 |
| Methylparaben | Preservative | 1 | 0.25 |
| Disodium EDTA (dissolvine Na2) | Chelating agent | 1 | 0.1 |
| Sodium carbomer (PNC-430) | Emulsion stabilizing agent, viscosity increasing | 2 | 1 |
| | agent | | |
| Ammonium Acryloyldimethyltaurate/VP | Viscosity increasing agent | 3 | 0.5 |
| copolymer (Aristoflex AVC) | | | |
| Octinoxate | Active sunscreen | 4 | 4 |
| Dimethicone, polysilicone-11 (gransil DMG- | Skin conditioning, skin protectant | 4 | 4.4 |
| 6) | | | |
| Phenyl trimethicone, polysilicone 11 (Gransil | Detackifying agent | 4 | 3.65 |
| PM gel) | | | |
| Squalane HQ (olive oil derived) | Hydrating agent, emollient | 4 | 1 |
| Tocopheryl Acetate (Vitamin E acetate) | Hydrating agent | 4 | 0.1 |
| Aluminum Benzoate, mica, Disodium | surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 0.8 |
| Propylparaben | Preservative | 4 | 0.1 |
| HDI/Trimethylol Hexyllactone Crosspolymer | Detackifying agent | 5 | 1 |
| (BPD-500/plastic powder D) | | | |
| Cyclomethicone, polysilicone-11 (Gransil | Detackifying agent | 6 | 4.4 |
| GCM) | | | |
| Cyclomethicone/cyclopentasiloxane (Dow | Detackifying agent | 7 | 2 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese | Anti-oxidant, oxygenation, DNA self-repair | 7 | 0.03 |
| chloride (Euk-134) | | | |
| Retinol (retinol 50c liquid + polysorbate 20) | Cellular renewal | 7 | 0.025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | preservative | 7 | 1 |
| Capryly/glycol, Potassium Sorbate, Water, | | | |
| Hexylene Glycol (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Example 8

Topical multi-target anti-age formulation with reduced retinol to decrease retinol to 0.0025% w/w to reduce irritation

| Ingredient (trademark) | Function(s) | Group # | % W/w |
|---|---|---|---|
| Water | Moisturizer | 1 | 63.3075 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 1 |
| | agent | | |
| Ammonium Acryloyldimethyltaurate/VP | Viscosity increasing agent | 3 | 0.5 |
| copolymer (Aristoflex AVC) | | | |
| Dimethicone, polysilicone-11 (gransil DMG- | Skin conditioning, skin protectant | 4 | 4.4 |
| 6) | | | |
| Phenyl trimethicone, polysilicone 11 (Gransil | Detackifying agent | 4 | 3.65 |
| PM gel) | | | |
| Squalane HQ (olive oil derived) | Hydrating agent, emollient | 4 | 3 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 0.8 |
| Propylparaben | Preservative | 4 | 0.1 |
| Caprylic/capric triglyceride (crodamol GTCC) | Hydrating agent | 4 | 2 |
| HDI/Trimethylol Hexyllactone Crosspolymer | Detackifying agent | 5 | 1 |
| (BPD-500/plastic powder D) | | | |
| Cyclomethicone, polysilicone-11 (Gransil | Detackifying agent | 6 | 4.4 |
| GCM) | | | |
| Cyclomethicone/cyclopentasiloxane (Dow | Detackifying agent | 7 | 2 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Epilobium Angustifolium extract | Anti-irritation agent | 7 | 2 |
| Ethylbisiminomethylguaiacol manganese | Anti-oxidant, oxygenation, DNA self-repair | 7 | 0.03 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| | | | 100 |

### Manufacturing Process

The composition was prepared by following the steps described in Example 1 above.

### Specifications

The pH was of 6.19+/−0.25 with a range of 5.94-6.44. The viscosity calculated on a Brookfield LVT Model DV I at 25 degrees Centigrade/1 minute, spindle #4 at 3 RPMs, dial reading at 66% was of 132,000 cps with a range of between about 125,000 to 140,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Accelerated Stability Testing

The testing was performed as described in Example 2 above.

The pH remained stable at all temperatures. Viscosity varied slightly but no liquefaction or excessive thickening was observed and no viscosity change was observed upon application on the skin. The fragrance was stable although its intensity progressively weakened over time. The fragrance started to change at 45° C. at 3 months, which could be equated, to about 2 years at 25° C. The colour at the surface of the cream darkened and the browning rate correlated to the temperature and light slightly accelerated browning. The colour remained stable however within the cream. It was estimated that the cream would become dark brown within two years %. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered stable with an expected life span of more than 2 years %.

### Example 9

Topical multi-target anti-age formulation with added hydrating agents (Moist and LNST)
to reduce skin dryness, and decreased sodium carbomer concentration to reduce viscosity

| Ingredient (trademark) | Function(s) | Group # | % W/w |
|---|---|---|---|
| Water | Moisturizer | 1 | 51.6675 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system | 1 | 2 |
| 15) | | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.4 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Alpha bisabolol racemic (bisabolol) | Anti-irritation agent, anti-inflammatory and | 4 | 0.02 |
| | antibacterial agent | | |
| Dipalmitoyl hydroxyproline (Sepilift DPHP) | Fibroblast relaxation (reduce expression wrinkles), | 4 | 1 |
| | anti MMP, anti-elastase, synthesis ofTIMP2, | | |
| | stimulates lamins, anti-oxidant, anti-inflammatory | | |
| Lignoceryl Erucate (crodamol LGE) | Skin conditioning | 4 | 3 |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Skin conditioning, Detackifying agent | 7 | 1 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.03 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | Hydrating agent | | |
| succinic triglyceride (LNST 98) | | | |
| Dipotassium Glycyrrhizate (Net DG) | Anti-inflammatory agent, Hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.35+/−0.25 with a range of 6.10-6.60. The viscosity calculated on a Brookfield LVT Model DV I at 25 degrees Centigrade/1 minute, spindle #4 at 6 RPMs, dial reading at 54% was of 54,100 cps with a range of between about 48,000 to 58,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.982-1.014.

### Accelerated Stability Testing

The testing was performed as described in Example 2 above.

The pH remained stable at all temperatures with a slight but acceptable decrease at 45° C. Viscosity decreased slightly but no liquefaction or excessive thickening was observed and no viscosity change was observed upon application on the skin. The decrease was of about 1000 cps per month at room temperature but stabilization at 8000 cps was expected within about 6 months to two years.

The fragrance was stable although its intensity progressively weakened over time. The fragrance started to change at 45° C. at 3 months, which could be equated, to about 2 years at 25° C. The colour at the surface of the cream darkened and the browning rate correlated to the temperature. The tone remained within beige tones however. It was estimated that the cream would become dark beige within two years ½. Emulsion remained stable at all temperatures without separation or syneresis between 4 and 45° C. The cream handled well sharp temperature changes. The cream showed a few water drops at the surface of the jars after freeze-thaw cycles but the emulsion did not separate. This cream was considered medium stable with an expected life span of about 1 year ½. It could reach about 3 years with a tolerance to darkening.

### Example 10

Topical multi-target anti-age formulation without Dipalmitoyl hydroxyproline

| Ingredient (trademark) | Function(s) | Group # | % w/w |
|---|---|---|---|
| Water | Moisturizer | 1 | 52.6675 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.4 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Alpha bisabolol racemic (bisabolol) | Anti-irritation agent, anti-inflammatory and | 4 | 0.02 |
| | antibacterial agent | | |
| Lignoceryl Erucate (crodamol LGE) | Skin conditioning | 4 | 3 |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.03 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | Hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 11

Topical multi-target anti-age formulation with decreased Ethylbisiminomethylguaiacol
manganese chloride as causing brown spots in formulation

| Ingredient (trademark) | Function(s) | Group # | % W/w |
|---|---|---|---|
| Water | Moisturizer | 1 | 51.6875 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.4 |
| copolymer (Aristoflex AVC) | | | |
| Dimethicone, polysilicone-11 (Gransil DMG- | Detackifying agent, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Lignoceryl Erucate (crodamol LGE) | Skin conditioning | 4 | 3 |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 3 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, H | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Clinical Trial

The Japanese firm Medical Research International Inc. under the supervision of the investigator Dr. Matsukura performed an efficacy study with the composition of Examples 11 above and 24 below. The study was also meant to test the composition on sensitive skin. A monocentric open-ended study was conducted on 25 healthy Japanese volunteers (aged 26-62). Japanese skin is known as one of the most sensitive skin types in the world.

The treatment involved two daily applications of either the serum of Examples 2 or of Example 21 to the eye contour, face and neck areas for a period varying from 1 to 4.6 months according to each subject (per their age and skin condition). The volunteers received a 30 ml facial serum of either Example 2 or of Example 21 and were instructed to use 3-4 press pumps per application.

All subjects were required to pursue their usual hygiene regimen and to add the above-cited serum to their other usual beauty care products including cleansing lotion, tonic lotion, moisturizing cream, day or night cream, mask and others.

The following parameters were observed: overall skin comfort, lines, skin tone clarity, skin moisture, skin's cell network. Observation of the skin was performed using three methods that were adapted to the desired parameters as follows: 1) overall skin comfort: subject self assessment; lines: subject self assessment and investigator notes; skin tone clarity: subject self assessment, investigator notes and before and after photos as back-up support, skin moisture subject self assessment; skin's cell network: subject self assessment and investigator notes. Observations were done at day 0 for all subjects and at month 1, 2, 3 or 4.6 according to each subject.

The self assessment results showed that 1) 80% of volunteers liked the serum texture, application and scent while 20% moderately liked the serum texture, application and scent; 2) 93% found the product easy to use while 7% found it moderately easy to use; 3) 62% found improvement in their skin network (texture), 31% found moderate improvement in their skin network and 7% found little or no improvement in their skin network; 4) 64% found improvement in their skin tone (clarity), 18% found moderate improvement in their skin tone and 18% found little or no improvement in their skin tone; 5) 84% found improvement in their skin moisture, 11% found moderate improvement and 5% found little improvement; 6) 50% found improvement of their overall skin comfort; 37% found moderate improvement and 13% found little improvement in their skin comfort; 7) 59% found improvement in their lines in some area of their face while 41% found little or no improvement of their lines; 8) 72% found the product to be effective 11% found the product moderately effective and 17% found the product had little efficacy. More specifically the following skin aging sign and skin disorder and conditions were reduced or treated in at least one subject: inflammation, acne-rosacea, rashes, peripheral circulation, devitalization, sagging, dull skin tone, spots, unstable blood flow, fine line, wrinkles, irregular skin tone, dilated or clogged pores, rosacea, acne, blotches, excess sebum, seborrhea and sebum retention.

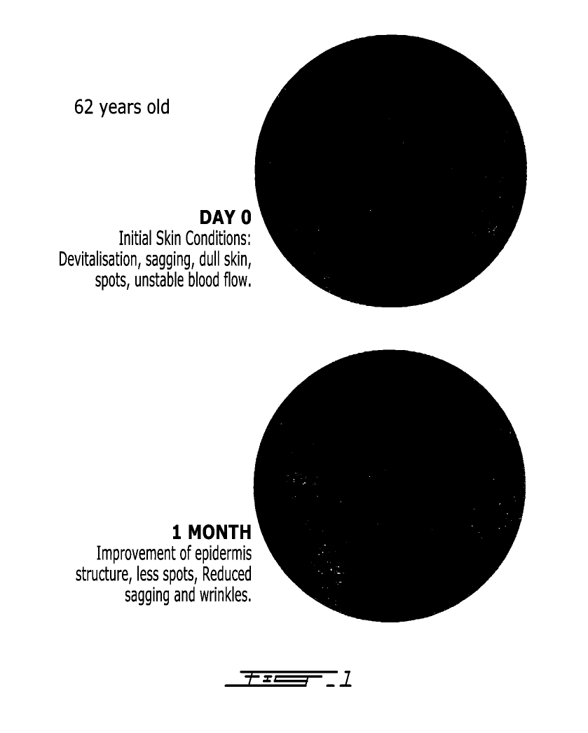

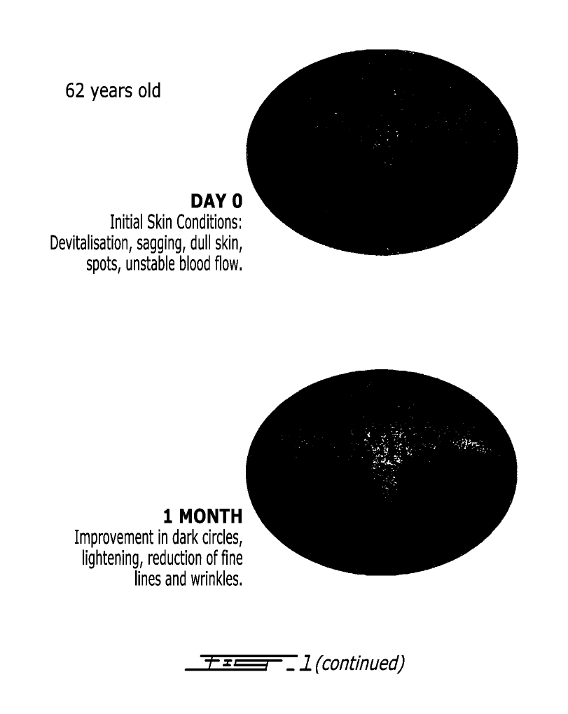

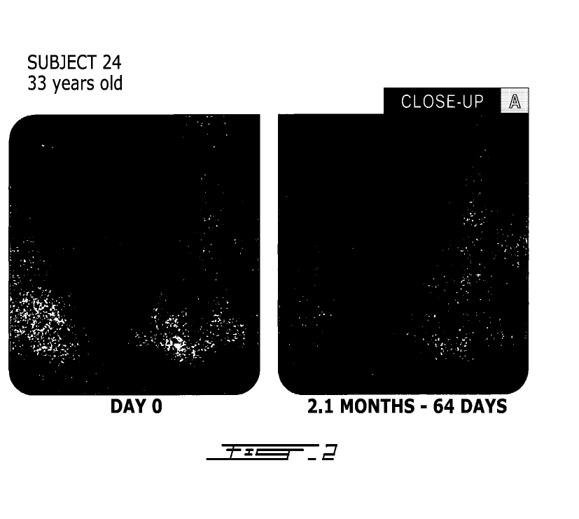

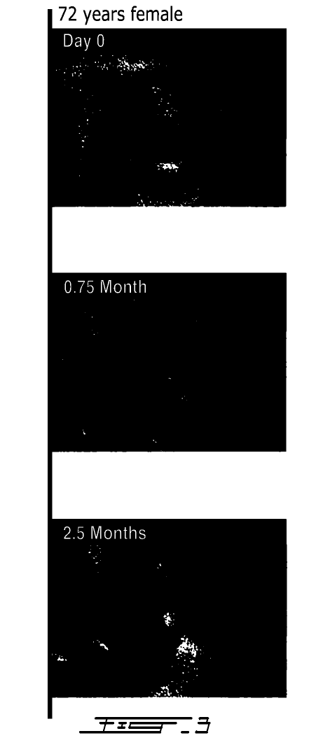

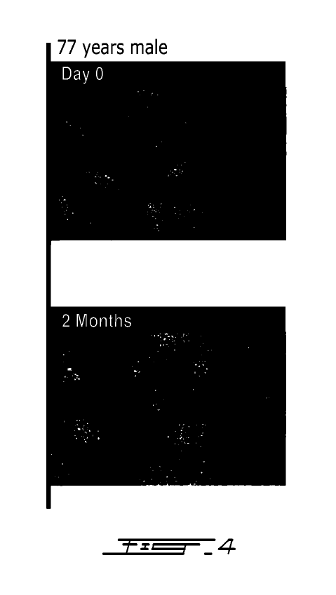

FIGS. 1 and 2 provide before and after pictures showing improvement of the skin. FIG. 3 presents a reduction over time of bags under eyes in a 72 years old women. FIG. 4 presents a reduction of sebum on the skin of a 77 years old man.

The report shows that the skin helps to improve the skin total balance, the appearance of the skin and the clarity (tone) of the skin. It lightens the skin and sometimes lightens brown spots when using the lighting formulation (composition of Example 21). Overall, the product has demonstrated its effectiveness in helping the peripheral microcirculation of lipids and blood, which is an important element in the elimination of toxins, sebum, and other impurities. It also plays a role toward improved microcapillary network thus reducing the risk of rosacea, blotches and other similar skin conditions associated with a disorganized micro-capillary network in the skin. Some subjects complained of skin irritation during the first few days of using the products, but the problem was soon settled. The report notes that most of the subjects were regular users of high-quality skincare and anti-aging facial treatments and that nevertheless, the regular application of the compositions of the present invention demonstrated significant skin improvements daily.

### Example 12

Topical multi-target anti-age formulation as in Example11 with scaled up process

| Ingredient (commercial name | | | |
|---|---|---|---|
| (corporation)) | Function(s) | Group # | % W/w |
| Water (Aqua) | Moisturizer | 1 (8%), 5 | 52.6875 |
| | | (46.270%) | |
| Glycerin 99% (Jeen) | Hydrating agent, skin protectant | 1 | 3.0000 |
| Retinol (retinol 50c liquid + polysorbate 20) | Cellular renewal | 1 | 0.0025 |
| Alteromonas Ferment Extract, Butylene | Anti-irritation agent, langherhans | 1 | 2.0000 |
| Glycol (Abyssine 657 (Atrium Lanatech)) | cell protection, skin matrix | | |
| | integrity | | |
| Imperata Cylindrica (Root Extract), Water, | Hydrating agent, osmosis | 1 | 3.0000 |
| Glycerin, PEG-8, Carbomer (Moist 24 | protection | | |
| (Sederma/Croda)) | | | |
| PEG-6 Isostearate, Hesperetin Laurate | Anti-elastase, vaso-regulatory, | 1 | 3.0000 |
| (Flavagrum PEG (Reference: ATNG946S) | anti-oxidant, anti-inflammatory, | | |
| (BASF)) | anti-puffiness | | |
| Propylene Glycol, Commiphora Myrrha | Hydrating agent | 1 | 0.1000 |
| Extract, Equisetum Arvense Extract, Triticum | | | |
| Vulgare (Wheat) Germ Extract, Humulus | | | |
| Lupulus (Hops) Extract (330-Regederme HS | | | |
| (Alban Muller)) | | | |
| Phenoxyethanol (preservative), | Preservative | 1 | 1.000 |
| Capryly/glycol, Potassium Sorbate, Water, | | | |
| Hexylene Glycol (Jeecide CAP-5) | | | |
| Dipotassium Glycyrrhizate (Net DG (Barnet)) | Anti-inflammatory agent, | 1 | 0.0200 |
| | Hydrating agent | | |
| Sesamum indicum seed oil, triticum vulgare | Anti-inflammatory agent, anti- | 1 | 2.0000 |
| germ oil, tocopheryl acetate, caprylic/capric | irritation agent, hydrating agent | | |
| succinic triglyceride (LNST 98) | | | |
| Hyaluronic Acid, water (hyasol BT 1%) | Hydrating agent | 1 | 2.0000 |
| (Centerchem)) | | | |
| Fragrance (Juniper Breeze #55472 | Produces or masks odours | 1 | 0.1500 |
| (Interome)) | | | |
| Glycosaminoglycans (MRTEX (Atrium)) | Anti-mmps; skin matrix integrity, | 1 | 2.0000 |
| | anti-inflammatory | | |
| Glycerin, butylene glycol, water, carbomer | Collagen synthesis | 1 | 3.0000 |
| (emulsion stabilizing agent), polysorbate-20, | | | |
| palmitoyl pentapeptide-4 (Matrixyl 3000) | | | |
| Creatine (TegoCosmo C-100 (Centerchem | ATP stimulation | 1 | 0.0100 |
| Inc.)) | | | |
| Cyclomethicone (Dow Corning 345 Fluid | Detackifying agent | 1 | 1.0000 |
| (Dow Corning)) | | | |
| Ethylbisiminomethylguaiacol Manganese | Anti-oxidant, oxygenation, DNA | 2 | 0.0100 |
| Chloride (EUK-134 (Atrium)) | self-repair | | |
| Sodium Carbomer (PNC-430 (3V Inc.)) | Emulsion stabilizing agent, | 3 (.3%), 6 | 0.8000 |
| | viscosity increasing agent | (.3%), 7 (.2%) | |
| Lignoceryl Erucate (crodamol LGE) | Skin conditioning | 4 | 3.0000 |
| Polysorbate-40 (Tween 40 (Uniquiema | Emulsifying agent | 4 | 1.0000 |
| America)) | | | |
| Tocopheryl Acetate (Vitamin E Acetate | Hydrating agent | 4 | 0.1000 |
| (Jeen)) | | | |
| Squalane (Squalane (Barnet)) | Hydrating agent, emollient | 4 | 4.0000 |
| Dipalmitoyl Hydroxyproline (Sepilift DPHP | Fibroblast relaxation (reduce | 4 | 1.0000 |
| (Seppic)) | expression wrinkles), anti MMP, | | |
| | anti-elastase, synthesis of TIMP2, | | |
| | stimulates lamins, anti-oxidant, | | |
| | anti-inflammatory | | |
| Aluminum Benzoate, Mica, Disodium | Surface modifier, UV light | 4 | 0.1000 |
| Distyrybiphenyl Disulfonate, Aluminum | absorber, cosmetic astringent | | |
| Chloride (Covazur Z03 (LCW)) | | | |
| Alpha bisabolol racemic (Bisabolol (Lipo)) | Anti-irritation agent, anti- | 4 | 0.0200 |
| | inflammatory and antibacterial | | |
| | agent | | |
| Phenyl Trimethicone (and) Polysilicone-11 | Detackifying agent | 4 | 3.2500 |
| (Gransil PM Gel (Grant Industries)) | | | |
| Propylparaben | Preservative | 4 | 0.1 |
| Dimethicone (and) Polysilicone-11 (Gransil | Detackifying agent, skin | 4 | 4.2000 |
| DMG-6 (Grant Industries)) | protectant | | |
| Disodium EDTA (Dissolvine Na2 (Akzo)) | Chelating agent | 5 | 0.1000 |
| PEG-8/SMDI Copolymer (Polyoprepolymer- | Controlled delivery system to | 5 | 2.0000 |
| 15 (Barnet)) | avoid irritation and inflammation | | |
| Ammonium Acryloyldimethyltaurate/VP | Viscosity increasing agent | 5 | 0.4000 |
| Copolymer (Aristoflex AVC (Clarient)) | | | |
| Methylparaben | Preservative | 5 | 0.25 |
| HDI/Trimethylol Hexyllactone Crosspolymer | Detackifying agent | 8 | 0.5000 |
| (BPD-500/Plastic Powder D (Kobo)) | | | |
| Cyclomethicone (and) Polysilicone-11 | Detackifying agent | 9 | 4.2000 |
| (Gransil GCM (Grant Industries)) | | | |
| | | | 100 |

### Manufacturing Process

1. In a stainless steel container, ingredients of group 1 as identified in the Table above were mixed together with a Greaves™ homogenizer. The mixture was agitated alternating the speed and direction between 5-6 FOR and 3-4 REV. The agitation was continued until a homogenous mixture was obtained.

2. While continuing the agitation described in step 1, the ingredient of group 2 (ethylbisiminomethylguaiacol) was sifted on the mixture with a weighting boat. The mixing time of steps 1 and 2 totalized about 40 minutes.

3. A first portion of the ingredients of group 3 (sodium carbomer) was slowly sifted (over about 20 minutes) in the mixture of step 2, while agitating with the Greaves™ homogenizer at 5+/−1 FOR. Mixing continued with the Greaves™ homogenizer and manually with a spatula until a homogenous mixture was obtained.

4. In a separate stainless steel container, ingredients of group 4 were mixed together with a Baldor™ agitator adjusted at 60 and heated to 80-82° C. Mixing continued until step 7.

5. The remaining amount of water was poured into a small Lee™ tank, and heated to 80-82° C. While agitating with the Leeson™ agitator and the Greaves™ homogenizer adjusted at 4-5, ingredients of group 5, except for the Ammonium Acryloyldimethyltaurate/VP copolymer, were added to the water. The Ammonium Acryloyldimethyltaurate/VP copolymer was then added slowly while frequently mixing with a spatula.

6. While agitating with the Leeson™ agitator, the Greaves™ homogenizer adjusted at 3-4 REV and while frequently manually agitating, the ingredient of group 6 (Sodium carbomer) was added to the mixture of step 5. Mixing was continued until a homogenous mixture was obtained. The mixing time of steps 5 and 6 totalized about 35 minutes.

7. When the temperature of the oily phase (step 4) reached about 80-82° C. and that of the water phase of step 6 reached about 76-78° C., the mixture of step 4 was combined to the mixture of step 6 while agitating with the Leeson™ agitator, the Greaves™ homogenizer adjusted at 4-5 FOR and manually with a spatula. The mixture was mixed until a complete emulsion was obtained.

8. While maintaining the agitation, the ingredient of group 7 (remaining amount of Sodium carbomer) was added slowly (over about 15 minutes) to the mixture of step 7. While the temperature was maintained at about 76-78° C., the Greaves™ homogenizer was adjusted at 4 REV, the mixture was agitated for a maximum of 15 minutes until a smooth homogenous mixture was obtained. The valve was drained twice during agitation (i.e. bottom portion of tank was drained and poured on top of batch to mix well).

9. While maintaining agitation, the mixture of step 8 was cooled down to about 68-70° C. When this temperature was reached while maintaining agitation by adjusting the Greaves™ homogenizer at 4-5 REV, the ingredient of group 8 was sifted into the mixture.

10. While maintaining agitation, the mixture of step 9 was cooled down to about 48-50° C. When this temperature was reached, the ingredient of group 9 was added. Mixing was continued for about 20 minutes until a homogenous mixture was obtained. The wall of the tank was manually agitated and the valve was drained during agitation.

11. While maintaining agitation, the mixture of group 10 was cooled down to about 45° C. When this temperature was reached, the mixture of step 3 was added while maintaining agitation. Mixing continued for about 10 minutes until a homogenous mixture was obtained. The wall of the tank was manually agitated and the valve was drained twice during agitation.

12. The Greaves™ homogenizer was stopped. Manual and Leeson agitations of the mixture of step 11 continued for about 15 minutes or until the mixture was cooled down to about 30+/−2 C.

This scaled up process is appropriate for use for the various formulation of the present invention herein.

### Accelerated Stability Testing

Upon reception, the samples were stored at 25° C. and 45° C. The appearance, odour, colour were measured at 2 weeks, 1, 2 and 3 months. Appearance: smooth, homogenous emulsion, no brown specks and no sign of separation were observed at 25 or 45° C. The pH was stable at both temperatures throughout the test.

The viscosity at 25° C. after 2 weeks dropped by 1500 cps, down to 11750 cps, and after 4 weeks had dropped by 2250 cps to 11000 cps. Thereafter the viscosity slowly decreased to 10250 cps during the second and third months. This is not significant since viscosity fluctuates over time.

Viscosity at 45° C. after 4 weeks had dropped by 5250 cps down to 8000 cps. The product is rated fairly stable without signs of emulsion separation. This product is expected to have a shelf life of about 2 years at room temperature.

### Clinical Trial

The safety and efficiency of the composition of Example 12 was tested on 124 volunteers of female gender in France mainland, 45 to 65 years old with every type of skin. The volunteers were instructed to use the skin care twice a day during 4 weeks by pushing 4 times on the pump for each application, to not used any other skin care on their face and to otherwise not change their cosmetic habits.

Some distinctive points have been identified by analyzing the data collected in the questionnaires of the 114 respondents and by comparing them to the corresponding data on the face skin care database of the Consumer studies firm containing data from 10842 skin care cases. This composition was established to be 1) very efficient in moisturizing the skin, namely significantly better in close-ended question than corresponding data in the database; 2) very efficient in leaving the skin comfortable, namely significantly better in open-ended question than corresponding data in the database; and 3) very efficient in reducing the wrinkles, namely significantly better in open-ended question than corresponding data in the database. The report also established that the safety profile of the composition was very good since the rate of discomfort was significantly lower than that in the database and the rate of tolerance was in accordance with that in the database.

### Example 13

Topical multi-target anti-age formulation with added viscosity increasing agent,
replaced lignoceryl by caprylic/capric triglyceride

| Ingredient (trademark) | Function(s) | Group # | % W/w |
|---|---|---|---|
| Water | Moisturizer | 1 | 52.5375 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.55 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduces expression wrinkles), | 4 | 1 |
| | anti MMP, anti-elastase, synthesis of TIMP2, | | |
| | stimulates lamins, anti-oxidant, anti-inflammatory | | |
| Caprylic/capric triglyceride (crodamol GTCC) | Hydrating agent | 4 | 3 |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Skin conditioning, Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 14

Topical multi-target anti-age formulation with added active hydrogenated polyisobutene,
anemarrhenae asphodeloides root extract (volufiline) 1% w/w, increased concentration
of viscosity agent

| Ingredient (trademark) | Function(s) | Group # | % W/w |
|---|---|---|---|
| Water | Moisturizer | 1 | 51.4875 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.6 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, anemarrhenae | Improves lipid content of skin and reduce wrinkle, | 4 | 1 |
| asphodeloides root extract (Volufiline) | cellular renewal | | |
| HDI/Trimethylol Hexyllactone Crosspolymer | Anticaking agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

At 4 and 25° C., this product's stability was good. At 45° C., the cream had become yellowish and caused a separation of the emulsion. The shelf life of this product is expected to be around 1.5 years at 25° C.

### Example 15

Topical multi-target anti-age formulation with added active hydrogenated
polyisobutene, anemarrhenae asphodeloides root extract (Volufiline ™) 3% w/w,
increased concentration of viscosity agent

| Ingredient (trademark) | Function(s) | Group # | % W/w |
|---|---|---|---|
| Water | Moisturizer | 1 | 49.4875 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.6 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, anemarrhenae | Improves lipid content of skin and reduce wrinkle, | 4 | 3 |
| asphodeloides root extract (Volufiline) | cellular renewal | | |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

This emulsion separated after 1 month at all temperatures.

### Example 16

Topical multi-target anti-age formulation with added active hydrogenated polyisobutene,
anemarrhenae asphodeloides root extract (Volufiline ™) 5% w/w, increased
concentration of viscosity agent

| Ingredient (trademark) | Function(s) | Group # | % W/w |
|---|---|---|---|
| Water | Moisturizer | 1 | 47.4875 |
| Glycerin | Hydrating agent, skin conditioning, skin protectant | 1 | 3 |
| Methylparaben | Preservative | 1 | 0.25 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.6 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Propylparaben | Preservative | 4 | 0.1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, anemarrhenae | Improves lipid content of skin and reduce wrinkle, | 4 | 5 |
| asphodeloides root extract (Volufiline) | cellular renewal | | |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

This emulsion separated after 1 month at all temperatures.

### Example 17

Deep Wrinkles-integral Corrective concentrate
Topical multi-target anti-age formulation without paraben or phenoxyethanol

| Ingredient (trademark) | Function(s) | Group # | % W/w |
|---|---|---|---|
| Water | Moisturizer | 1 | 51.6375 |
| Glycerin | Hydrating agent, skin conditioning, skin protectant | 1 | 3 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.6 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | UV light absorber | 4 | 0.1 |
| Distyrybiphenyl Disulfonate, Aluminum | | | |
| Chloride (Covazur Z03) | | | |
| Polysorbate-40 (Tween 40) | Emulsifying agent | 4 | 1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, anemarrhenae | Improves lipid content of skin and reduce wrinkle, | 4 | 1 |
| asphodeloides root extract (Volufiline) | cellular renewal | | |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produce or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| Sodium metabisulfite, disodium pyrosulfite, | Anti-browning agent | 7 | 0.2 |
| disodium disulfite, sodium pyrosulfite | | | |
| 1,2-hexanediol, caprylyl glycol | Preservative | 7 | 1 |
| (Symdiol 68) | | | |
| | | | 100 |

### Manufacturing Process

Steps 1 to 8 described in Example 1 above were performed.

9. The batch was then run through an inline homogenizer, insert drop-in homogenizer or a high sheer turbine-mixing unit to improve the appearance of the emulsion and compensate for water lost during processing (i.e. addition of 3 to 5% extra water during step 1 to compensate water loss during manufacturing as is standard in the industry). If the emulsion became slightly inverted after the final phase addition, the mixture was homogenized to obtain a smooth and even texture.

10. The mixture was cooled down to 25° C. and mixing was continued until the mixture was uniform.

### Specifications

The pH of the composition was 6.27+/−0.25 with a range of 6.50 to 6.02.

The viscosity calculated on a Brookfield LVT Model DV I at 25 degrees Centigrade/1 minute, spindle #4 at 6 RPMs, dial reading at 45% was of 45,200 cps with a range of about 42,000 to 52,000 cps. The color was off white to slightly beige. The odor was characteristic to slightly fruity. The appearance was that of a moderately viscous cream with a smooth texture. The specific gravity was of 1.002+/−0.02 on a stainless steel grease pychnometer at 25 degrees centigrade with a range of 0.982-1.022.

### Example 18

Topical multi-target anti-age formulation without paraben and hydrogenated
polyisobutene, anemarrhenae asphodeloides root extract

| Ingredient (trademark) | Function(s) | Group # | % W/w |
|---|---|---|---|
| Water | Moisturizer | 1 | 52.6375 |
| Glycerin | Hydrating agent, skin conditioning, skin protectant | 1 | 3 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.6 |
| copolymer (Aristoflex AVC) | | | |
| Dimethicone, polysilicone-11 (gransil DMG- | Skin conditioning, skin protectant | 4 | 4.2 |
| 6) | | | |
| Phenyl trimethicone, polysilicone 11 (Gransil | Detackifying agent | 4 | 3.25 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.1 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| HDI/Trimethylol Hexyllactone Crosspolymer | Detackifying agent | 5 | 0.5 |
| (BPD-500/plastic powder D) | | | |
| Cyclomethicone, polysilicone-11 (Gransil | Detackifying agent | 6 | 4.2 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produce or masks odours | 7 | 0.15 |
| Glycerin, butylene glycol, water, carbomer | Collagen synthesis | 7 | 3 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Phenoxyethanol (preservative), | Preservative | 7 | 1 |
| Capryly/glycol, Potassium Sorbate, Water, | | | |
| Hexylene Glycol (Jeecide CAP-5) | | | |
| Sesamum indicum seed oil, triticum vulgare | Anti-inflammatory agent, anti-irritation agent, | 7 | 2 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 2 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 3 |
| (Flavagrum PEG) | inflammatory, anti-puffiness | | |
| Sodium metabisulfite, disodium pyrosulfite, | Anti-browning agent | 7 | 0.2 |
| disodium disulfite, sodium pyrosulfite | | | |
| | | | 100 |

### Manufacturing Process

The composition was prepared by following the steps described in Example 17 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 19

Anti-wrinkles and firming-integral corrective serum
Topical multi-target anti-age formulation without paraben or phenoxyethanol,
with added actives

| Ingredient (trademark) | Function(s) | Group # | % W/w |
|---|---|---|---|
| Water | Moisturizer | 1 | 49.6275 |
| Glycerin | Hydrating agent, skin conditioning, skin protectant | 1 | 3 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| PVP polyvinylpyrrolidone | Film forming agents | 1 | 0.2 |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.55 |
| copolymer (Aristoflex AVC) | | | |
| Dimethicone, polysilicone-11 (gransil DMG- | Skin conditioning, skin protectant | 4 | 2.1 |
| 6) | | | |
| Phenyl trimethicone, polysilicone 11 (Gransil | Detackifying agent | 4 | 1.6 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.15 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Caprylic/capric triglyceride (crodamol | Hydrating agent | 4 | 2 |
| GTCC) | | | |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, | Improves lipid content of skin and reduce wrinkle, | 4 | 1 |
| anemarrhenae asphodeloides root extract | cellular renewal | | |
| (Volufiline) | | | |
| Nylon-12 (Orgasol 2002 (Lipo)) | Light diffracting agent | 5 | 0.5 |
| Cyclomethicone, polysilicone-11 (Gransil | Detackifying agent | 6 | 2.1 |
| GCM) | | | |
| Cyclomethicone/cyclopentasiloxane (Dow | Skin conditioning, Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Fragrance (Juniper Breeze) | Produces or masks odours | 7 | 0.15 |
| Glycerin, butylene glycol, water, carbomer | Collagen synthesis | 7 | 1 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Sesamum indicum seed oil, triticum vulgare | Anti-inflammatory agent, anti-irritation agent, | 7 | 1 |
| germ oil, tocopheryl acetate, caprylic/capric | Hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 1 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 1 |
| (Flavagrum PEG) | inflammatory, anti-puffiness | | |
| Ceramide 3, 6 II, 1, Phytosphingosine, | Hydrating agent, Inhibition of tyrosinase activity | 7 | 3 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 7 | 1 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman | Improves skin barrier function, anti-oxidant | 7 | 0.05 |
| 6) | | | |
| Water, butylene glycol, dextran, palmitoyl | Anti-inflammatory | 7 | 0.5 |
| tripeptide-8 (Neutrazen) | | | |
| Ascophyllum nodosum extract, sorbitol, | Cellular renewal | 7 | 0.5 |
| water (Homeo age) | | | |
| Fucus serratus extract, glycerol (Homeo | Improves skin barrier function | 7 | 0.5 |
| shield) | | | |
| Ascophyllum nodosum extract, sorbitol, | Cellular renewal | 7 | 0.5 |
| water (Homeo soothe) | | | |
| Ubiquinone (coenzyme Q10) | Cellular renewal | 7 | 0.01 |
| Water, dextran, acetyl tetrapeptide-2 | Reinforcing the cutaneous immune defences and | 7 | 1 |
| (Thymulen 4 PS 100) | cellular renewal | | |
| 1,2-hexanediol, caprylyl glycol (Symdiol 68) | Hydrating agent, preservative | 7 | 1 |
| Water, acetyl octapeptide-3 (snap-8 | Cellular renewal | 8 | 1 |
| solution) | | | |
| Sodium DNA from Sturgeon (HDPR, | Inhibition of tyrosinase activity, hydrating agent, | 8 | 0.5 |
| Javanech) | Cellular renewal, anti-aging, anti-wrinkle | | |
| Salix Alba bark extract (NAB willow bark | Cellular renewal | 8 | 5 |
| extract) | | | |
| Sorbitol, yeast extract (Dryline) | Hydrating agent | 9 | 0.1 |
| Water, glycerin, rumex occidentalis extract, | Inhibition of tyrosinase | 10 | 1 |
| rumex crispus root extract, rumex | | | |
| pseudonatronatus, rumex steonophyllus, | | | |
| rumex wallichii extract (tyrostat) | | | |
| Sodium metabisulfite, disodium pyrosulfite, | Anti-browning agent | 11 | 0.2 |
| disodium disulfite, sodium pyrosulfite | | | |
| | | | 100 |

### Manufacturing Process

The composition was prepared following steps 1 to 9 described in Example 17 above.

10. The ingredients of group # 8 were then pre-mixed in a different kettle under a high energy/propeller mixer, until these ingredients were completely dissolved and they were combined to the mixture of groups 1-7. When the ingredients of group 8 were added to the batch, the top and bottom were observed to ensure that the batch was well formed and uniform.

11. The ingredients of groups 9, 10 and 11 were then added each by each to the batch while cooling batch.

12. The mixture was cooled down to 25° C. and mixing was continued until uniform.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 20

Multi-target Anti-wrinkle Eye-Integral corrective serum
Topical anti-age formulation without paraben or phenoxyethanol, with anti-ingredients increased and
stimulating ingredients reduced

| Ingredient (trademark) | Function(s) | Group # | % W/w |
|---|---|---|---|
| Water | Moisturizer | 1 | 45.6475 |
| Glycerin | Hydrating agent, skin conditioning, skin protectant | 1 | 3 |
| Disodium EDTA (dissolvine Na2) | Chelating agent | 1 | 0.1 |
| PEG-8/SMDI Copolymer (polyoprepolymer- | Controlled delivery system to avoid irritation and | 1 | 2 |
| 15) | inflammation | | |
| Sodium carbomer (PNC-430) | Emulsion stabilizing agent, viscosity increasing | 2 | 0.8 |
| | agent | | |
| Ammonium Acryloyldimethyltaurate/VP | Viscosity increasing agent | 3 | 0.55 |
| copolymer (Aristoflex AVC) | | | |
| Dimethicone, polysilicone-11 (gransil DMG- | Skin conditioning, skin protectant | 4 | 2.1 |
| 6) | | | |
| Phenyl trimethicone, polysilicone 11 (Gransil | Detackifying agent | 4 | 1.5 |
| PM gel) | | | |
| Squalane HQ (olive oil derived) | Hydrating agent, emollient | 4 | 4 |
| Tocopheryl Acetate (Vitamin E acetate) | Hydrating agent | 4 | 0.1 |
| Aluminum Benzoate, mica, Disodium | Surface modifier, UV light absorber, cosmetic | 4 | 0.15 |
| Distyrybiphenyl Disulfonate, Aluminum | astringent | | |
| Chloride (Covazur Z03) | | | |
| Polysorbate-40 (Tween 40) | Emulsifying agent | 4 | 1 |
| Caprylic/capric triglyceride (crodamol GTCC) | Hydrating agent | 4 | 3 |
| Alpha bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Hydrogenated polyisobutene, anemarrhenae | Improves lipid content of skin and reduce wrinkle, | 4 | 0.01 |
| asphodeloides root extract (Volufiline) | cellular renewal | | |
| Nylon-12 (Orgasol 2002 (Lipo)) | Light diffracting agent | 5 | 2 |
| Cyclomethicone, polysilicone-11 (Gransil | Detackifying agent | 6 | 2.1 |
| GCM) | | | |
| Cyclomethicone/cyclopentasiloxane (Dow | Detackifying agent | 7 | 1 |
| corning 345 fluid) | | | |
| Glycosaminoglycans (MRTEX complex) | Anti-mmps; skin matrix integrity, anti-inflammatory | 7 | 2 |
| Hyaluronic Acid, water (hyasol BT 1%) | Hydrating agent | 7 | 2 |
| Ethylbisiminomethylguaiacol manganese | Anti-oxidant, oxygenation, DNA self-repair | 7 | 0.01 |
| chloride (Euk-134) | | | |
| Retinol (retinol 50c liquid + polysorbate 20) | Cellular renewal | 7 | 0.0025 |
| Creatine (TegoCosmo c-100) | ATP stimulation | 7 | 0.01 |
| Glycerin, butylene glycol, water, carbomer | Collagen synthesis | 7 | 0.01 |
| (emulsion stabilizing agent), polysorbate-20, | | | |
| palmitoyl pentapeptide-4 (Matrixyl 3000) | | | |
| Propylene Glycol (humectant, solvent), | Hydrating agent | 7 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Sesamum indicum seed oil, triticum vulgare | Anti-inflammatory agent, anti-irritation agent, | 7 | 0.03 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 7 | 0.02 |
| Alteromonas ferment extract, butylene glycol | Anti-irritation agent, langherhans cell protection, | 7 | 0.02 |
| (Abyssine 657) | skin matrix integrity | | |
| Imperata Cylindrica root extract, water, | Hydrating agent, osmosis protection | 7 | 3 |
| Glycerin, PEG-8, Carbomer (Moist 24) | | | |
| PEG-6 isostearate, hesperetin laurate | Anti-elastase, vaso-regulatory, anti-oxidant, anti- | 7 | 0.75 |
| (Flavagrum PEG) | inflammatory, anti-puffiness | | |
| Ceramide 3, 6 II, 1, Phytosphingosine, | Hydrating agent, Inhibition of tyrosinase activity | 7 | 5 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 7 | 0.01 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman | Improves skin barrier function | 7 | 0.05 |
| 6) | | | |
| Water, butylene glycol, dextran, palmitoyl | Anti-inflammatory | 7 | 0.05 |
| tripeptide-8 (Neutrazen) | | | |
| Ascophyllum nodosum extract, sorbitol, | Cellular renewal | 7 | 0.05 |
| water (Homeo age) | | | |
| Fucus serratus extract, glycerol (Homeo | Improves skin barrier function | 7 | 0.05 |
| shield) | | | |
| Ascophyllum nodosum extract, sorbitol, | Cellular renewal | 7 | 0.05 |
| water (Homeo soothe) | | | |
| Ubiquinone (coenzyme Q10) | Cellular renewal | 7 | 0.01 |
| Water, dextran, acetyl tetrapeptide-2 | Reinforcing the cutaneous immune defences, | 7 | 0.05 |
| (Thymulen 4 PS 100) | cellular renewal | | |
| Water, sorbitol, ascophyllum nodosum | Skin Matrix integrity (anti-angiogenesis) | 7 | 2 |
| extract, asparagopsis armata extract | | | |
| (aldavine) | | | |
| Water, butylene glycol, Ahnfeltia Concinna | Hydrating agent, cellular renewal | 7 | 2 |
| Extract (APT) | | | |
| Hydrolyzed Rice Bran Protein, Glycine Soja | Anti-puffiness | 7 | 2 |
| protein, Oxido reductases (Regu-age) | | | |
| Mehyldihydrojasminate (Hedione) | Produces or masks odours | 7 | 0.25 |
| 1,2-hexanediol, caprylyl glycol (Symdiol 68) | Hydrating agent, preservative | 7 | 1 |
| Water, acetyl octapeptide-3 (snap-8 | Cellular renewal | 8 | 2 |
| solution) | | | |
| Sodium DNA from Sturgeon (HDPR, | Inhibition of tyrosinase activity, hydrating agent, | 8 | 1 |
| Javanech) | Cellular renewal, anti-aging, anti-wrinkle | | |
| Salix Alba bark extract (NAB willow bark | Cellular renewal | 8 | 5 |
| extract) | | | |
| Sodium metabisulfite, disodium pyrosulfite, | Anti-browning agent | 9 | 0.2 |
| disodium disulfite, sodium pyrosulfite | | | |
| Citrus Dulcis Auritrusantium (Orange0 Peel | Produces or masks odours | 10 | 1.2 |
| Oil (+limonene) as allergene for EU | | | |
| | | | 100 |

### Manufacturing Process

The composition was prepared following steps described in Example 19 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 21

Topical anti-age formulation with whitening agent with active sunscreen

| Ingredient (commercial name (corporation)) | Function(s) | Group # | % W/w |
|---|---|---|---|
| Water (Aqua) | Moisturizer | 1 | 67.309 |
| Disodium EDTA (Dissolvine Na2 (Akzo)) | Chelating agent | 1 | 0.1000 |
| Phenoxyethanol (preservative), Capryly/glycol, | Preservative | 1 | 1 |
| Potassium Sorbate, Water, Hexylene Glycol | | | |
| (Jeecide CAP-5) | | | |
| Sodium Carbomer (PNC-430 (3V Inc.)) | Emulsion stabilizing agent, viscosity | 2 | 1.0000 |
| | increasing agent | | |
| Ammonium Acryloyldimethyltaurate/VP | Viscosity increasing agent | 3 | 0.5000 |
| Copolymer (Aristoflex AVC (Clarient)) | | | |
| Octinoxate | Active sunscreen | 4 | 4 |
| Polysorbate-40 (Tween 40 (Uniquiema America)) | Emulsifying agent | 4 | 0.8000 |
| Tocopheryl Acetate (Vitamin E Acetate (Jeen)) | Hydrating agent | 4 | 0.1000 |
| Squalane (Squalane (Barnet)) | Hydrating agent, emollient | 4 | 1.0000 |
| Aluminum Benzoate, Mica, Disodium | Surface modifier, UV light absorber, | 4 | 0.1000 |
| Distyrybiphenyl Disulfonate, Aluminum Chloride | cosmetic astringent | | |
| (Covazur Z03 (LCW)) | | | |
| Phenyl Trimethicone (and) Polysilicone-11 | Detackifying agent | 4 | 3.6500 |
| (Gransil PM Gel (Grant Industries)) | | | |
| Propylparaben | Preservative | 4 | 0.1 |
| Dimethicone (and) Polysilicone-11 (Gransil DMG- | Detackifying agent, skin protectant | 4 | 4.4000 |
| 6 (Grant Industries)) | | | |
| HDI/Trimethylol Hexyllactone Crosspolymer (BPD- | Detackifying agent | 5 | 1.0000 |
| 500/Plastic Powder D (Kobo)) | | | |
| Methylparaben | Preservative | 4 | 0.25 |
| Cyclomethicone (and) Polysilicone-11 (Gransil | Detackifying agent | 6 | 4.4000 |
| GCM (Grant Industries)) | | | |
| Epilobium Angustifolium extract | Anti-irritation agent | 7 | 2 |
| Hyaluronic Acid, water (hyasol BT 1%) | Hydrating agent | 7 | 2.0000 |
| (Centerchem)) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 3.0000 |
| | inflammatory | | |
| Sodium Ascorbyl Phosphate | Skin matrix integrity, collagen synthesis, | 7 | 0.0010 |
| | inhibition of tyrosinase activity, anti- | | |
| | oxidant | | |
| Creatine (TegoCosmo C-100 (Centerchem Inc.)) | ATP stimulation | 7 | 0.0100 |
| Cyclomethicone (Dow Corning 345 Fluid (Dow | Detackifying agent | 7 | 2.0000 |
| Corning)) | | | |
| Ethylbisiminomethylguaiacol Manganese Chloride | Anti-oxidant, oxygenation, DNA self-repair | 7 | 0.0300 |
| (EUK-134 (Atrium)) | | | |
| Rumex Occidentalis Extract, Ascorbic acid | Inhibition of tyrosinase | 7 | 1.0000 |
| (Tyrostat (Atrium)) | | | |
| Propylene Glycol, Commiphora Myrrha Extract, | Hydrating agent | 7 | 0.1000 |
| Equisetum Arvense Extract, Triticum Vulgare | | | |
| (Wheat) Germ Extract, Humulus Lupulus (Hops) | | | |
| Extract (330-Regederme HS (Alban Muller)) | | | |
| | | | 100 |

### Manufacturing Process

The composition was prepared following steps described in Example 1 above.

### Specifications

The pH of the composition was of 6.26+/−0.25 with a range of 6.01-6.51. The viscosity calculated on a Brookfield LVT Model DV I at 25 degrees Centigrade/1 minute, spindle #4 at 3 RPMs, dial reading at 58% was of 116,000 cps with a range of between about 112,000 and 130,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Accelerated Stability Testing

Testing and results were as reported in Example 2 above.

### Example 22

Topical anti-age formulation with whitening agent with increased squalane, added PEG-8/SMDI Copolymer
and caprylic/capric triglyceride

| Ingredient (commercial name (corporation)) | Function(s) | Group # | % W/w |
|---|---|---|---|
| Water (Aqua) | Moisturizer | 1 | 65.309 |
| Disodium EDTA (Dissolvine Na2 (Akzo)) | Chelating agent | 1 | 0.1000 |
| PEG-8/SMDI Copolymer (Polyoprepolymer-15 | Controlled delivery system to avoid | 1 | 2.0000 |
| | irritation and inflammation | | |
| (Barnet)) | | | |
| Phenoxyethanol (preservative), Capryly/glycol, | Preservative | 1 | 1 |
| Potassium Sorbate, Water, Hexylene Glycol | | | |
| (Jeecide CAP-5) | | | |
| Sodium Carbomer (PNC-430 (3V Inc.)) | Emulsion stabilizing agent, viscosity | 2 | 1.0000 |
| | increasing agent | | |
| Ammonium Acryloyldimethyltaurate/VP | Viscosity increasing agent | 3 | 0.5000 |
| Copolymer (Aristoflex AVC (Clarient)) | | | |
| Caprylic/Capric Triglyceride (Crodamol GTCC | Hydrating agent | 4 | 2.0000 |
| (Croda)) | | | |
| Polysorbate-40 (Tween 40 (Uniquiema | Emulsifying agent | 4 | 0.8000 |
| America)) | | | |
| Tocopheryl Acetate (Vitamin E Acetate (Jeen)) | Hydrating agent | 4 | 0.1000 |
| Squalane (Squalane (Barnet)) | Hydrating agent, emollient | 4 | 3.0000 |
| Aluminum Benzoate, Mica, Disodium | Surface modifier, UV light absorber, | 4 | 0.1000 |
| Distyrybiphenyl Disulfonate, Aluminum Chloride | cosmetic astringent | | |
| (Covazur Z03 (LCW)) | | | |
| Phenyl Trimethicone (and) Polysilicone-11 | Detackifying agent | 4 | 3.6500 |
| (Gransil PM Gel (Grant Industries)) | | | |
| Propylparaben | Preservative | 4 | 0.1 |
| Dimethicone (and) Polysilicone-11 (Gransil | Detackifying agent, skin protectant | 4 | 4.4000 |
| DMG-6 (Grant Industries)) | | | |
| HDI/Trimethylol Hexyllactone Crosspolymer | Detackifying agent | 5 | 1.0000 |
| (BPD-500/Plastic Powder D (Kobo)) | | | |
| Methylparaben | Preservative | 4 | 0.25 |
| Cyclomethicone (and) Polysilicone-11 (Gransil | Skin conditioning, detackifying agent | 6 | 4.4000 |
| GCM (Grant Industries)) | | | |
| Epilobium Angustifolium extract | Anti-irritation agent | 7 | 2 |
| Hyaluronic Acid, water (hyasol BT 1%) | Hydrating agent | 7 | 2.0000 |
| (Centerchem)) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 3.0000 |
| | inflammatory | | |
| Sodium Ascorbyl Phosphate | Skin matrix integrity, collagen synthesis, | 7 | 0.0010 |
| | inhibition of tyrosinase activity, anti- | | |
| | oxidant | | |
| Creatine (TegoCosmo C-100 (Centerchem | ATP stimulation | 7 | 0.0100 |
| Inc.)) | | | |
| Cyclomethicone (Dow Corning 345 Fluid (Dow | Detackifying agent | 7 | 2.0000 |
| Corning)) | | | |
| Ethylbisiminomethylguaiacol Manganese | Anti-oxidant, oxygenation, DNA self- | 7 | 0.0300 |
| Chloride (EUK-134 (Atrium)) | repair | | |
| Rumex Occidentalis Extract, Ascorbic acid | Inhibition of tyrosinase | 7 | 1.0000 |
| (Tyrostat (Atrium)) | | | |
| Propylene Glycol, Commiphora Myrrha Extract, | Hydrating agent | 7 | 0.1000 |
| Equisetum Arvense Extract, Triticum Vulgare | | | |
| (Wheat) Germ Extract, Humulus Lupulus (Hops) | | | |
| Extract (330-Regederme HS (Alban Muller)) | | | |
| | | | 100 |

### Manufacturing Process

The composition was prepared by following the steps described in Example 1 above.

### Specifications

The pH was of 6.26+/−0.25 with a range of 6.01-6.51. The viscosity calculated on a Brookfield LVT Model DV I at 25 degrees Centigrade/1 minute, spindle #4 at 3 RPMs, dial reading at 58% was of 116,000 cps with a range of between about 112,000 to 130,000 cps. The color was off white to slightly beige. The odor was characteristic to slightly fruity. The appearance was that of a moderately viscous cream with a smooth texture. The specific gravity was of 1.006+/−0.02 a stainless steel grease pychnometer at 25 degrees centigrade with a range of 0.986-1.026.

### Accelerated Stability Testing

The testing was performed as described in Example 2 above.

The pH remained stable at all temperatures. Viscosity varied slightly but no liquefaction or excessive thickening was observed and no viscosity change was observed upon application on the skin. The fragrance was stable although its intensity progressively weakened over time. The fragrance started to change at 45° C. at 3 months, which could be equated, to about 2 years at 25° C. The colour at the surface of the cream darkened and the browning rate correlated to the temperature and light slightly accelerated browning. It was estimated that the cream would become medium brown within two years %. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered weakly stable with an expected life span of not more than 2 years.

### Example 23

Topical anti-age formulation with whitening agent, with added anti-aging agents, reduced dimethicone and
polysilicone-11; reduced cyclomethicone/cyclopentasiloxane, reduced Cyclomethicone and polysilicone-11,
increased polysorbate 40, increased squalane, reduced Aristoflex, sodium carbomer, BPD-50, added
glycerin, removed Epilobium angustifolium extract

| Ingredient (commercial name (corporation)) | Function(s) | Group # | % W/w |
|---|---|---|---|
| Water (Aqua) | Moisturizer | 1 | 53.669 |
| Glycerin 99% (Jeen) | Hydrating agent, skin conditioning, skin | 1 | 3.0000 |
| | protectant | | |
| Disodium EDTA (Dissolvine Na2 (Akzo)) | Chelating agent | 1 | 0.1000 |
| PEG-8/SMDI Copolymer (Polyoprepolymer-15 | Controlled delivery system to avoid | 1 | 2.0000 |
| (Barnet)) | irritation and inflammation | | |
| Phenoxyethanol (preservative), Capryly/glycol, | Preservative | 1 | 1.000 |
| Potassium Sorbate, Water, Hexylene Glycol | | | |
| (Jeecide CAP-5) | | | |
| Sodium Carbomer (PNC-430 (3V Inc.)) | Emulsion stabilizing agent, viscosity | 2 | 0.8000 |
| | increasing agent | | |
| Ammonium Acryloyldimethyltaurate/VP | Viscosity increasing agent | 3 | 0.4000 |
| Copolymer (Aristoflex AVC (Clarient)) | | | |
| Lignoceryl Erucate (crodamol LGE) | Skin conditioning | 4 | 3.0000 |
| Polysorbate-40 (Tween 40 (Uniquiema America)) | Emulsifying agent | 4 | 1.0000 |
| Tocopheryl Acetate (Vitamin E Acetate (Jeen)) | Hydrating agent | 4 | 0.1000 |
| Squalane (Squalane (Barnet)) | Hydrating agent, emollient | 4 | 4.0000 |
| Dipalmitoyl Hydroxyproline (Sepilift DPHP | Fibroblast relaxation (reduces expression | 4 | 1.0000 |
| (Seppic)) | wrinkles), anti MMP, anti-elastase, | | |
| | synthesis of TIMP2, stimulates lamins, | | |
| | anti-oxidant, anti-inflammatory | | |
| Aluminum Benzoate, Mica, Disodium | Surface modifier, UV light absorber, | 4 | 0.1000 |
| Distyrybiphenyl Disulfonate, Aluminum Chloride | cosmetic astringent | | |
| (Covazur Z03 (LCW)) | | | |
| Alpha bisabolol racemic (Bisabolol (Lipo)) | Anti-irritation agent, anti-inflammatory | 4 | 0.0200 |
| | and antibacterial agent | | |
| Phenyl Trimethicone (and) Polysilicone-11 | Detackifying agent | 4 | 3.2500 |
| (Gransil PM Gel (Grant Industries)) | | | |
| Propylparaben | Preservative | 4 | 0.1 |
| Dimethicone (and) Polysilicone-11 (Gransil DMG- | Detackifying agent, skin protectant | 4 | 4.2000 |
| 6 (Grant Industries)) | | | |
| HDI/Trimethylol Hexyllactone Crosspolymer | Detackifying agent | 5 | 0.5000 |
| (BPD-500/Plastic Powder D (Kobo)) | | | |
| Methylparaben | Preservative | 4 | 0.25 |
| Cyclomethicone (and) Polysilicone-11 (Gransil | Detackifying agent | 6 | 4.2000 |
| GCM (Grant Industries)) | | | |
| Sesamum indicum seed oil, triticum vulgare germ | Anti-inflammatory agent, anti-irritation | 7 | 2.0000 |
| oil, tocopheryl acetate, caprylic/capric succinic | agent, hydrating agent | | |
| triglyceride (LNST 98) | | | |
| Dipotassium Glycyrrhizate (Net DG (Barnet)) | Anti-inflammatory agent, hydrating agent | 7 | 0.0200 |
| Hyaluronic Acid, water (hyasol BT 1%) | Hydrating agent | 7 | 2.0000 |
| (Centerchem) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 3.0000 |
| | inflammatory | | |
| Sodium Ascorbyl Phosphate | Skin matrix integrity, collagen synthesis, | 7 | 0.0010 |
| | inhibition of tyrosinase activity, anti- | | |
| | oxidant | | |
| Creatine (TegoCosmo C-100 (Centerchem Inc.)) | ATP stimulation | 7 | 0.0100 |
| Cyclomethicone (Dow Corning 345 Fluid (Dow | Skin conditioning, Detackifying agent | 7 | 1.0000 |
| Corning)) | | | |
| Ethylbisiminomethylguaiacol Manganese Chloride | Anti-oxidant, oxygenation, DNA self- | 7 | 0.0300 |
| (EUK-134 (Atrium)) | repair | | |
| Rumex Occidentalis Extract, Ascorbic acid | Inhibition of tyrosinase | 7 | 1.0000 |
| (Tyrostat (Atrium)) | | | |
| Alteromonas Ferment Extract, Butylene Glycol | Anti-irritation agent, langherhans cell | 7 | 2.0000 |
| (Abyssine 657 (Atrium Lanatech)) | protection, skin matrix integrity | | |
| Imperata Cylindrica (Root Extract), Water, | Hydrating agent, osmosis protection | 7 | 3.0000 |
| Glycerin, PEG-8, Carbomer (Moist 24 | | | |
| (Sederma/Croda)) | | | |
| PEG-6 Isostearate, Hesperetin Laurate | Anti-elastase, vaso-regulatory, anti- | 7 | 3.0000 |
| (Flavagrum PEG (Reference: ATNG946S) | oxidant, anti-inflammatory, anti-puffiness | | |
| (BASF)) | | | |
| Propylene Glycol, Commiphora Myrrha Extract, | Hydrating agent | 7 | 0.1000 |
| Equisetum Arvense Extract, Triticum Vulgare | | | |
| (Wheat) Germ Extract, Humulus Lupulus (Hops) | | | |
| Extract (330-Regederme HS (Alban Muller)) | | | |
| | | | 100 |

### Manufacturing Process

The composition was prepared by following the steps described in Example 1 above.

### Specifications

The pH was of 6.25+/−0.25 with a range of 6.00-6.50. The viscosity calculated on a Brookfield LVT Model DV I at 25 degrees Centigrade/1 minute, spindle #4 at 6 RPMs, dial reading at 68% was of 62,700 cps with a range of between about 55,000 to 65,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Accelerated Stability Testing

The testing was performed as described in Example 2 above.

The pH remained stable at all temperatures with a slight but acceptable decrease at 45° C. Viscosity decreased slightly but no liquefaction or excessive thickening was observed and no viscosity change was observed upon application on the skin. The decrease was of about 1000 cps per month at room temperature but stabilization at 8000 cps was expected within about 6 months to two years.

The fragrance was stable although its intensity progressively weakened over time. The fragrance started to change at 45° C. at 3 months, which could be equated, to about 2 years at 25° C. The colour at the surface of the cream darkened and the browning rate correlated to the temperature. Light did not accelerate browning. The tone became brown at 45° C. It was estimated that the cream would become beige within one years and brown witan two years at 20° C. Emulsion remained stable at all temperatures without separation or syneresis between 4 and 45° C. The cream showed a few water drops at the surface of the jars after freeze-thaw cycles but the emulsion did not separate. This cream was considered medium stable with an expected life span of about 1 year. It could reach about 2 years ½ with a tolerance to darkening.

### Example 24

Topical anti-age formulation with whitening agent, without Dipalmitoyl hydroxyproline

| Ingredient (commercial name (corporation)) | Function(s) | Group # | % W/w |
|---|---|---|---|
| Water (Aqua) | Moisturizer | 1 | 54.669 |
| Glycerin 99% (Jeen) | Hydrating agent, skin conditioning, | 1 | 3.0000 |
| | skin protectant | | |
| Disodium EDTA (Dissolvine Na2 (Akzo)) | Chelating agent | 1 | 0.1000 |
| PEG-8/SMDI Copolymer (Polyoprepolymer-15 | Controlled delivery system to avoid | 1 | 2.0000 |
| (Barnet)) | irritation and inflammation | | |
| Phenoxyethanol (preservative), Capryly/glycol, | Preservative | 1 | 1.0000 |
| Potassium Sorbate, Water, Hexylene Glycol | | | |
| (Jeecide CAP-5) | | | |
| Sodium Carbomer (PNC-430 (3V Inc.)) | Emulsion stabilizing agent, viscosity | 2 | 0.8000 |
| | increasing agent | | |
| Ammonium Acryloyldimethyltaurate/VP Copolymer | Viscosity increasing agent | 3 | 0.4000 |
| (Aristoflex AVC (Clarient)) | | | |
| Lignoceryl Erucate (crodamol LGE) | Skin conditioning | 4 | 3.0000 |
| Polysorbate-40 (Tween 40 (Uniquiema America)) | Emulsifying agent | 4 | 1.0000 |
| Tocopheryl Acetate (Vitamin E Acetate (Jeen)) | Hydrating agent | 4 | 0.1000 |
| Squalane (Squalane (Barnet)) | Hydrating agent, emollient | 4 | 4.0000 |
| Aluminum Benzoate, Mica, Disodium | Surface modifier, UV light absorber, | 4 | 0.1000 |
| Distyrybiphenyl Disulfonate, Aluminum Chloride | cosmetic astringent | | |
| (Covazur Z03 (LCW)) | | | |
| Alpha bisabolol racemic (Bisabolol (Lipo)) | Anti-irritation agent, anti-inflammatory | 4 | 0.0200 |
| | and antibacterial agent | | |
| Phenyl Trimethicone (and) Polysilicone-11 (Gransil | Detackifying agent | 4 | 3.2500 |
| PM Gel (Grant Industries)) | | | |
| Propylparaben | Preservative | 4 | 0.1 |
| Dimethicone (and) Polysilicone-11 (Gransil DMG-6 | Detackifying agent, skin protectant | 4 | 4.2000 |
| (Grant Industries)) | | | |
| HDI/Trimethylol Hexyllactone Crosspolymer (BPD- | Detackifying agent | 5 | 0.5000 |
| 500/Plastic Powder D (Kobo)) | | | |
| Methylparaben | Preservative | 4 | 0.25 |
| Cyclomethicone (and) Polysilicone-11 (Gransil | Detackifying agent | 6 | 4.2000 |
| GCM (Grant Industries)) | | | |
| Sesamum indicum seed oil, triticum vulgare germ | Anti-inflammatory agent, anti-irritation | 7 | 2.0000 |
| oil, tocopheryl acetate, caprylic/capric succinic | agent, hydrating agent | | |
| triglyceride (LNST 98) | | | |
| Dipotassium Glycyrrhizate (Net DG (Barnet)) | Anti-inflammatory agent, hydrating | 7 | 0.0200 |
| | agent | | |
| Hyaluronic Acid, water (hyasol BT | Hydrating agent | 7 | 2.0000 |
| 1%)(Centerchem) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 3.0000 |
| | inflammatory | | |
| Sodium Ascorbyl Phosphate | Skin matrix integrity, collagen | 7 | 0.0010 |
| | synthesis, inhibition of tyrosinase | | |
| | activity, anti-oxidant | | |
| Creatine (TegoCosmo C-100 (Centerchem Inc.)) | ATP stimulation | 7 | 0.0100 |
| Cyclomethicone (Dow Corning 345 Fluid (Dow | Detackifying agent | 7 | 1.0000 |
| Corning)) | | | |
| Ethylbisiminomethylguaiacol Manganese Chloride | Anti-oxidant, oxygenation, DNA self- | 7 | 0.0300 |
| (EUK-134 (Atrium)) | repair | | |
| Rumex Occidentalis Extract, Ascorbic acid | Inhibition of tyrosinase | 7 | 1.0000 |
| (Tyrostat (Atrium)) | | | |
| Alteromonas Ferment Extract, Butylene Glycol | Anti-irritation agent, langherhans cell | 7 | 2.0000 |
| (Abyssine 657 (Atrium Lanatech)) | protection, skin matrix integrity | | |
| Imperata Cylindrica (Root Extract), Water, Glycerin, | Hydrating agent, osmosis protection | 7 | 3.0000 |
| PEG-8, Carbomer (Moist 24 (Sederma/Croda)) | | | |
| PEG-6 Isostearate, Hesperetin Laurate (Flavagrum | Anti-elastase, vaso-regulatory, anti- | 7 | 3.0000 |
| PEG (Reference: ATNG946S) (BASF)) | oxidant, anti-inflammatory, anti- | | |
| | puffiness | | |
| Propylene Glycol, Commiphora Myrrha Extract, | Hydrating agent | 7 | 0.1000 |
| Equisetum Arvense Extract, Triticum Vulgare | | | |
| (Wheat) Germ Extract, Humulus Lupulus (Hops) | | | |
| Extract (330-Regederme HS (Alban Muller)) | | | |
| | | | 100 |

### Manufacturing Process

The composition was prepared by following the steps described in Example 1 above.

### Specifications

The pH was of 6.25+/−0.25 with a range of 6.00-6.50. The viscosity calculated on a Brookfield LVT Model DV I at 25 degrees Centigrade/1 minute, spindle #4 at 6 RPMs, dial reading at 35% was of 35,400 cps with a range of between about 32,000 to 42,000 cps. The color was off white to slightly beige. The odor was characteristic to slightly fruity. The appearance was that of a moderately viscous cream with a smooth texture. The specific gravity was of 1.001+/−0.02 a stainless steel grease pychnometer at 25 degrees centigrade with a range of 0.981-1.021.

### Example 25

Topical anti-age formulation with whitening agent, with dipalmitoyl hydroxyproline, reduced
Ethylbisiminomethylguaiacol Manganese Chloride to remove brown spots, reduced glycosaminoglycans
with scaled up process

| Ingredient (commercial name (corporation)) | Function(s) | Group # | % W/w |
|---|---|---|---|
| Water (Aqua) | Moisturizer | 1 (8%), 5 | 54.6890 |
| | | (46.68 | |
| | | 90%) | |
| Glycerin 99% (Jeen) | Hydrating agent, skin conditioning, | 1 | 3.0000 |
| | skin protectant | | |
| Rumex Occidentalis Extract, Ascorbic acid | Inhibition of tyrosinase | 1 | 1.0000 |
| (Tyrostat (Atrium)) | | | |
| Alteromonas Ferment Extract, Butylene Glycol | Anti-irritation agent, langherhans cell | 1 | 2.0000 |
| (Abyssine 657 (Atrium Lanatech)) | protection, skin matrix integrity | | |
| Imperata Cylindrica (Root Extract), Water, | Hydrating agent, osmosis protection | 1 | 3.0000 |
| Glycerin, PEG-8, Carbomer (Moist 24 | | | |
| (Sederma/Croda)) | | | |
| PEG-6 Isostearate, Hesperetin Laurate | Anti-elastase, vaso-regulatory, anti- | 1 | 3.0000 |
| (Flavagrum PEG (Reference: ATNG946S) | oxidant, anti-inflammatory, anti- | | |
| (BASF)) | puffiness | | |
| Propylene Glycol, Commiphora Myrrha | Hydrating agent | 1 | 0.1000 |
| Extract, Equisetum Arvense Extract, Triticum | | | |
| Vulgare (Wheat) Germ Extract, Humulus | | | |
| Lupulus (Hops) Extract (330-Regederme HS | | | |
| (Alban Muller)) | | | |
| Phenoxyethanol (preservative), Capryly/glycol, | Preservative | 1 | 1 |
| Potassium Sorbate, Water, Hexylene Glycol | | | |
| (Jeecide CAP-5) | | | |
| Dipotassium Glycyrrhizate (Net DG (Barnet)) | Anti-inflammatory agent, hydrating | 1 | 0.0200 |
| | agent | | |
| Caprylic/Capric Triglyceride (Crodamol GTCC | Hydrating agent | 1 | 2.0000 |
| (Croda)) | | | |
| Hyaluronic Acid, water (hyasol BT 1%) | Hydrating agent | 1 | 2.0000 |
| (Centerchem) | | | |
| Fragrance (Juniper Breeze #55472 (Interome)) | Produces or masks odours | 1 | 0.1500 |
| Glycosaminoglycans (MRTEX (Atrium)) | Anti-mmps; skin matrix integrity, anti- | 1 | 2.0000 |
| | inflammatory | | |
| Sodium Ascorbyl Phosphate | Skin matrix integrity, collagen | 1 | 0.0010 |
| | synthesis, inhibition of tyrosinase | | |
| | activity, anti-oxidant | | |
| Creatine (TegoCosmo C-100 (Centerchem | ATP stimulation | 1 | 0.0100 |
| Inc.)) | | | |
| Cyclomethicone (Dow Corning 345 Fluid (Dow | Detackifying agent | 1 | 1.0000 |
| Corning)) | | | |
| Ethylbisiminomethylguaiacol Manganese | Anti-oxidant, oxygenation, DNA self- | 2 | 0.0100 |
| Chloride (EUK-134 (Atrium)) | repair | | |
| Sodium Carbomer (PNC-430 (3V Inc.)) | Emulsion stabilizing agent, viscosity | 3 | 0.8000 |
| | increasing agent | (.3%), 6 | |
| | | (.3%), | |
| | | 7 (.2%) | |
| Lignoceryl Erucate (crodamol LGE) | Skin conditioning | 4 | 3.0000 |
| Polysorbate-40 (Tween 40 (Uniquiema | Emulsifying agent | 4 | 1.0000 |
| America)) | | | |
| Tocopheryl Acetate (Vitamin E Acetate (Jeen)) | Hydrating agent | 4 | 0.1000 |
| Squalane (Squalane (Barnet)) | Hydrating agent, emollient | 4 | 4.0000 |
| Dipalmitoyl Hydroxyproline (Sepilift DPHP | Fibroblast relaxation (reduce | 4 | 1.0000 |
| (Seppic)) | expression wrinkles), anti MMP, anti- | | |
| | elastase, synthesis ofTIMP2, | | |
| | stimulates lamins, anti-oxidant, anti- | | |
| | inflammatory | | |
| Aluminum Benzoate, Mica, Disodium | Surface modifier, UV light absorber, | 4 | 0.1000 |
| Distyrybiphenyl Disulfonate, Aluminum | cosmetic astringent | | |
| Chloride (Covazur Z03 (LCW)) | | | |
| Alpha bisabolol racemic (Bisabolol (Lipo)) | Anti-irritation agent, anti-inflammatory | 4 | 0.0200 |
| | and antibacterial agent | | |
| Phenyl Trimethicone (and) Polysilicone-11 | Detackifying agent | 4 | 3.2500 |
| (Gransil PM Gel (Grant Industries)) | | | |
| Propylparaben | Preservative | 4 | 0.1 |
| Dimethicone (and) Polysilicone-11 (Gransil | Detackifying agent, skin protectant | 4 | 4.2000 |
| DMG-6 (Grant Industries)) | | | |
| Disodium EDTA (Dissolvine Na2 (Akzo)) | Chelating agent | 5 | 0.1000 |
| PEG-8/SMDI Copolymer (Polyoprepolymer-15 | Controlled delivery system to avoid | 5 | 2.0000 |
| (Barnet)) | irritation and inflammation | | |
| Ammonium Acryloyldimethyltaurate/VP | Viscosity increasing agent | 5 | 0.4000 |
| Copolymer (Aristoflex AVC (Clarient)) | | | |
| Methylparaben | Preservative | 5 | 0.25 |
| HDI/Trimethylol Hexyllactone Crosspolymer | Detackifying agent | 8 | 0.5000 |
| (BPD-500/Plastic Powder D (Kobo)) | | | |
| Cyclomethicone (and) Polysilicone-11 (Gransil | Detackifying agent | 9 | 4.2000 |
| GCM (Grant Industries)) | | | |
| | | | 100 |

### Manufacturing Process

The composition was prepared following process steps described in Example 12 above.

### Specifications

As described in Example 23 above.

### Accelerated Stability Testing

Upon reception, the samples were stored at 25° C. and 45° C. The appearance, odour, colour were measured at 2 weeks, 1, 2 and 3 months. Appearance: smooth, homogenous emulsion, no brown specks and no sign of separation were observed at 25 or 45° C. The pH was stable at both temperatures throughout the test.

The viscosity at 25° C. after 4 weeks had dropped by 2250 cps to 10500 cps. Thereafter the viscosity slowly decreased to 9000 cps during the second and third months. This is not significant since viscosity fluctuates over time.

Viscosity at 45° C. after 4 weeks had dropped by 6000 cps down to 7000 cps. Thereafter the viscosity slowly decreased to 6250 cps during the second and third months. The product is rated fairly stable without signs of emulsion separation. This product is expected to have a shelf life of about 2 years at room temperature.

### Example 26

Topical anti-age formulation with whitening agent, with Crodamol GTCC instead of lignoceryl and increased
Ammonium Acryloyldimethyltaurate/VP Copolymer

| Ingredient (commercial name (corporation)) | Function(s) | Group # | % W/w |
|---|---|---|---|
| Water (Aqua) | Moisturizer | 1 | 54.539 |
| Glycerin 99% (Jeen) | Hydrating agent, skin conditioning, skin | 1 | 3.0000 |
| | protectant | | |
| Disodium EDTA (Dissolvine Na2 (Akzo)) | Chelating agent | 1 | 0.1000 |
| Methylparaben | Preservative | 1 | 0.25 |
| PEG-8/SMDI Copolymer (Polyoprepolymer-15 | Controlled delivery system to avoid | 1 | 2.0000 |
| (Barnet)) | irritation and inflammation | | |
| Sodium Carbomer (PNC-430 (3V Inc.)) | Emulsion stabilizing agent, viscosity | 2 | 0.8000 |
| | increasing agent | | |
| Ammonium Acryloyldimethyltaurate/VP | Viscosity increasing agent | 3 | 0.5500 |
| Copolymer (Aristoflex AVC (Clarient)) | | | |
| Caprylic/Capric Triglyceride (Crodamol GTCC | Hydrating agent | 4 | 3.0000 |
| (Croda)) | | | |
| Polysorbate-40 (Tween 40 (Uniquiema | Emulsifying agent | 4 | 1.0000 |
| America)) | | | |
| Tocopheryl Acetate (Vitamin E Acetate (Jeen)) | Hydrating agent | 4 | 0.1000 |
| Squalane (Squalane (Barnet)) | Hydrating agent, emollient | 4 | 4.0000 |
| Dipalmitoyl Hydroxyproline (Sepilift DPHP | Fibroblast relaxation (reduce expression | 4 | 1.0000 |
| (Seppic)) | wrinkles), anti MMP, anti-elastase, | | |
| | synthesis of TIMP2, stimulates lamins, anti- | | |
| | oxidant, anti-inflammatory | | |
| Aluminum Benzoate, Mica, Disodium | Surface modifier, UV light absorber, | 4 | 0.1000 |
| Distyrybiphenyl Disulfonate, Aluminum | cosmetic astringent | | |
| Chloride (Covazur Z03 (LCW)) | | | |
| Propylparaben | Preservative | 4 | 0.1 |
| Alpha bisabolol racemic (Bisabolol (Lipo)) | Anti-irritation agent, anti-inflammatory and | 4 | 0.0200 |
| | antibacterial agent | | |
| Phenyl Trimethicone (and) Polysilicone-11 | Detackifying agent | 4 | 3.2500 |
| (Gransil PM Gel (Grant Industries)) | | | |
| Dimethicone (and) Polysilicone-11 (Gransil | Detackifying agent, skin protectant | 4 | 4.2000 |
| DMG-6 (Grant Industries)) | | | |
| HDI/Trimethylol Hexyllactone Crosspolymer | Detackifying agent | 5 | 0.5000 |
| (BPD-500/Plastic Powder D (Kobo)) | | | |
| Cyclomethicone (and) Polysilicone-11 (Gransil | Detackifying agent | 6 | 4.2000 |
| GCM (Grant Industries)) | | | |
| Dipotassium Glycyrrhizate (Net DG (Barnet)) | Anti-inflammatory agent, hydrating agent | 7 | 0.0200 |
| Hyaluronic Acid, water (hyasol BT 1%) | Hydrating agent | 7 | 2.0000 |
| (Centerchem) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 2.0000 |
| | inflammatory | | |
| Sodium Ascorbyl Phosphate | Skin matrix integrity, collagen synthesis, | 7 | 0.0010 |
| | inhibition of tyrosinase activity, anti-oxidant | | |
| Creatine (TegoCosmo C-100 (Centerchem | ATP stimulation | 7 | 0.0100 |
| Inc.)) | | | |
| Cyclomethicone (Dow Corning 345 Fluid (Dow | Detackifying agent | 7 | 1.0000 |
| Corning)) | | | |
| Ethylbisiminomethylguaiacol Manganese | Anti-oxidant, oxygenation, DNA self-repair | 7 | 0.0100 |
| Chloride (EUK-134 (Atrium)) | | | |
| Rumex Occidentalis Extract, Ascorbic acid | Inhibition of tyrosinase | 7 | 1.0000 |
| (Tyrostat (Atrium)) | | | |
| Sesamum indicum seed oil, triticum vulgare | Anti-inflammatory agent, anti-irritation | 7 | 2.0000 |
| germ oil, tocopheryl acetate, caprylic/capric | agent, hydrating agent | | |
| succinic triglyceride (LNST 98) | | | |
| Alteromonas Ferment Extract, Butylene Glycol | Anti-irritation agent, langherhans cell | 7 | 2.0000 |
| (Abyssine 657 (Atrium Lanatech)) | protection, skin matrix integrity | | |
| Imperata Cylindrica (Root Extract), Water, | Hydrating agent, osmosis protection | 7 | 3.0000 |
| Glycerin, PEG-8, Carbomer (Moist 24 | | | |
| (Sederma/Croda)) | | | |
| PEG-6 Isostearate, Hesperetin Laurate | Anti-elastase, vaso-regulatory, anti-oxidant, | 7 | 3.0000 |
| (Flavagrum PEG (Reference: ATNG946S) | anti-inflammatory, anti-puffiness | | |
| (BASF)) | | | |
| Propylene Glycol, Commiphora Myrrha | Hydrating agent | 7 | 0.1000 |
| Extract, Equisetum Arvense Extract, Triticum | | | |
| Vulgare (Wheat) Germ Extract, Humulus | | | |
| Lupulus (Hops) Extract (330-Regederme HS | | | |
| (Alban Muller)) | | | |
| Phenoxyethanol (preservative), Capryly/glycol, | Preservative | 7 | 1 |
| Potassium Sorbate, Water, Hexylene Glycol | | | |
| (Jeecide CAP-5) | | | |
| | | | 100 |

### Manufacturing Process

The composition was prepared by following the steps described in Example 1 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 27

First wrinkles-integral prevention serum
Topical anti-age formulation with whitening agent, without paraben or
phenoxyethanol with scaled up process

| Ingredient (commercial name (corporation)) | Function(s) | Group # | % W/w |
|---|---|---|---|
| Water (Aqua) | Moisturizer | 1 | 54.639 |
| Glycerin 99% (Jeen) | Hydrating agent, skin conditioning, | 1 | 3.0000 |
| | skin protectant | | |
| Disodium EDTA (Dissolvine Na2 (Akzo)) | Chelating agent | 1 | 0.1000 |
| PEG-8/SMDI Copolymer (Polyoprepolymer-15 | Controlled delivery system | 1 | 2.0000 |
| (Barnet)) | | | |
| Sodium Carbomer (PNC-430 (3V Inc.)) | Emulsion stabilizing agent, viscosity | 2 | 0.8000 |
| | increasing agent | | |
| Ammonium Acryloyldimethyltaurate/VP Copolymer | Viscosity increasing agent | 3 | 0.6000 |
| (Aristoflex AVC (Clarient)) | | | |
| Caprylic/Capric Triglyceride (Crodamol GTCC | Hydrating agent | 4 | 3.0000 |
| (Croda)) | | | |
| Polysorbate-40 (Tween 40 (Uniquiema America)) | Emulsifying agent | 4 | 1.0000 |
| Tocopheryl Acetate (Vitamin E Acetate (Jeen)) | Hydrating agent | 4 | 0.1000 |
| Squalane (Squalane (Barnet)) | Hydrating agent, emollient | 4 | 4.0000 |
| Dipalmitoyl Hydroxyproline (Sepilift DPHP (Seppic)) | Fibroblast relaxation (reduce | 4 | 1.0000 |
| | expression wrinkles), anti MMP, anti- | | |
| | elastase, synthesis of TIMP2, | | |
| | stimulates lamins, anti-oxidant, anti- | | |
| | inflammatory | | |
| Aluminum Benzoate, Mica, Disodium | Surface modifier, UV light absorber, | 4 | 0.1000 |
| Distyrybiphenyl Disulfonate, Aluminum Chloride | cosmetic astringent | | |
| (Covazur Z03 (LCW)) | | | |
| Alpha bisabolol racemic (Bisabolol (Lipo)) | Anti-irritation agent, anti-inflammatory | 4 | 0.0200 |
| | and antibacterial agent | | |
| Phenyl Trimethicone (and) Polysilicone-11 (Gransil | Detackifying agent | 4 | 3.2500 |
| PM Gel (Grant Industries)) | | | |
| Dimethicone (and) Polysilicone-11 (Gransil DMG-6 | Detackifying agent, skin protectant | 4 | 4.2000 |
| (Grant Industries)) | | | |
| HDI/Trimethylol Hexyllactone Crosspolymer (BPD- | Detackifying agent | 5 | 0.5000 |
| 500/Plastic Powder D (Kobo)) | | | |
| Cyclomethicone (and) Polysilicone-11 (Gransil | Detackifying agent | 6 | 4.2000 |
| GCM (Grant Industries)) | | | |
| Dipotassium Glycyrrhizate (Net DG (Barnet)) | Anti-inflammatory active, hydrating | 7 | 0.0200 |
| | agent | | |
| Hyaluronic Acid, water (hyasol BT | Hydrating agent | 7 | 2.0000 |
| 1%)(Centerchem) | | | |
| Fragrance (Juniper Breeze #55472 (Interome)) | Produces or masks odours | 7 | 0.1500 |
| Glycosaminoglycans (MRTEX (Atrium)) | Anti-mmps; skin matrix integrity, anti- | 7 | 2.0000 |
| | inflammatory | | |
| Sodium Ascorbyl Phosphate | Skin matrix integrity, collagen | 7 | 0.0010 |
| | synthesis, inhibition of tyrosinase | | |
| | activity, anti-oxidant | | |
| Creatine (TegoCosmo C-100 (Centerchem Inc.)) | ATP stimulation | 7 | 0.0100 |
| Cyclomethicone (Dow Corning 345 Fluid (Dow | Detackifying agent | 7 | 1.0000 |
| Corning)) | | | |
| Ethylbisiminomethylguaiacol Manganese Chloride | Anti-oxidant, oxygenation, DNA self- | 7 | 0.0100 |
| (EUK-134 (Atrium)) | repair | | |
| Rumex Occidentalis Extract, Ascorbic acid | Inhibition of tyrosinase | 7 | 1.0000 |
| (Tyrostat (Atrium)) | | | |
| Sesamum indicum seed oil, triticum vulgare germe | Anti-inflammatory active, anti-irritation | 7 | 2.0000 |
| oil, tocopheryl acetate, caprylic/capric succinic | agent, hydrating agent | | |
| triglyceride (LNST 98 (Atrium/Lanatech)) | | | |
| Alteromonas Ferment Extract, Butylene Glycol | Anti-irritation agent, langherhans cell | 7 | 2.0000 |
| (Abyssine 657 (Atrium Lanatech)) | protection, skin matrix integrity | | |
| Imperata Cylindrica (Root Extract), Water, Glycerin, | Hydrating agent, osmosis protection | 7 | 3.0000 |
| PEG-8, Carbomer (Moist 24 (Sederma/Croda)) | | | |
| PEG-6 Isostearate, Hesperetin Laurate (Flavagrum | Anti-elastase, vaso-regulatory, anti- | 7 | 3.0000 |
| PEG (Reference: ATNG946S) (BASF)) | oxidant, anti-inflammatory, anti- | | |
| | puffiness | | |
| Propylene Glycol, Commiphora Myrrha Extract, | Hydrating agent | 7 | 0.1000 |
| Equisetum Arvense Extract, Triticum Vulgare | | | |
| (Wheat) Germ Extract, Humulus Lupulus (Hops) | | | |
| Extract (330-Regederme HS (Alban Muller)) | | | |
| 1,2-Hexanediol (and) Caprylyl glycol (Symdiol 68 | Hydrating agent, preservative | 7 | 1.0000 |
| (Symrise)) | | | |
| Sodium Metabisulfite (Sodium Metabisulfite | Anti-browning agent | 7 | 0.2000 |
| (Spectrum)) | | | |
| | | | 100 |

### Manufacturing Process

The composition was prepared following steps described in Example 17 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 66% was of 45,200 cps with a range of between about 42,000-52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 28

Anti-cellulite formulation

| Ingredient | Commercial name (corporation) | Group # | % W/w |
|---|---|---|---|
| Water (Aqua) | Water | 1 | 53.9800 |
| Glycerin | Glycerin 99% (Jeen) | 1 | 3.0000 |
| Disodium EDTA | Dissolvine Na2 (Akzo) | 1 | 0.1000 |
| Glycosaminoglycans | MDI Complex | 1 | 1.0000 |
| PEG-8/SMDI Copolymer (polyoprepolymer-15) | Delivery system | 1 | 2.0000 |
| Sodium Carbomer | PNC-430 (3V Inc.) | 2 | 0.8000 |
| Ammonium Acryloyldimethyltaurate/VP | Aristoflex AVC (Clarient) | 3 | 0.5500 |
| Copolymer | | | |
| Caprylic/Capric Triglyceride | Crodamol GTCC (Croda) | 4 | 2.0000 |
| Polysorbate-40 | Tween 40 (Uniquiema America) | 4 | 1.0000 |
| Tocopheryl Acetate | Vitamin E Acetate (Jeen) | 4 | 0.1000 |
| Squalane | Squalane (Barnet) | 4 | 4.0000 |
| Alpha bisabolol racemic | Bisabolol (Lipo) | 4 | 0.0200 |
| Fragrance | Juniper Breeze #55472 (Interome) | 7 | 0.1500 |
| Sesamum indicum seed oil, triticum vulgare | LNST 98 (Atrium/Lanatech) | 7 | 1.0000 |
| germe oil, tocopheryl acetate, caprylic/capric | | | |
| succinic triglyceride | | | |
| Alteromonas Ferment Extract, Butylene Glycol | Abyssine 657 (Atrium Lanatech) | 7 | 1.0000 |
| Imperata Cylindrica (Root Extract), Water, | Moist 24 (Sederma/Croda) | 7 | 3.0000 |
| Glycerin, PEG-8, Carbomer | | | |
| 1,2-Hexanediol, Caprylyl glycol | Symdiol 68 (Symrise) | 7 | 1.0000 |
| Water (Aqua), Pseudoalteromonas Ferment | Trylagen PCB (code: PD100) | 7 | 1.0000 |
| Extract, Hydrolyzed Wheat Protein, Hydrolyzed | (Centerchem) | | |
| Soy Protein, Tripeptide-10 Citrullinr, Triprptide- | | | |
| 1, Lecithin, Xanthan Gum, | | | |
| Carbomer, Triethanolamine | | | |
| Salix Nigra (Willow) Bark Extract | Willow Bark Extract | 8 | 5.0000 |
| Glaucine | Bodyfit | 8 | 3.0000 |
| Liex Paraguariensis | Unisilm | 8 | 3.0000 |
| | Pro Sveltyl Pearlchem | 8 | 3.0000 |
| | Liporeductyl Centerchem | 8 | 3.0000 |
| Palmitoyl Pentapeptide-4 | Matrixyl | 8 | 1.0000 |
| Peg 6 Isostearate | Flavagrum PEG | 8 | 1.000 |
| Carnitine | | 8 | 0.500 |
| Caffeine | | 8 | 0.500 |
| CoEnzyme A | | 8 | 0.500 |
| Esculin | | 8 | 0.500 |
| Sambacus Nigra Flower Extract | | 8 | 0.500 |
| Glycoprotein Panax Ginseng root extract | | 8 | 0.500 |
| Hydrolized Citrus Aurantium Dulcis Fruit Extract | Orange Extract (Silab) | 8 | 0.500 |
| Equisetum Arvense (horsetail extract) | | 8 | 0.500 |
| Bupleurum Salcatium Extract | | 8 | 0.500 |
| Unicarria Tomentosa Extract | | 8 | 0.500 |
| Sodium Metabisulphite | Sodium Matabisulfite (Spectrum) | 9 | 0.2000 |
| Sorbitol and Yeast extract (Faex in Europe) | Dryline (Atrium) | 10 | 0.1000 |
| | | | 100 |

### Manufacturing Process

The composition was prepared following steps described in Example 19 above.

### Specifications

The pH was of 6.27+/−0.25 with a range of 6.50-6.02. The viscosity calculated on a Brookfield LVT Model DV I at 25 degrees Centigrade/1 minute, spindle #4 at 6 RPMs, dial reading at 45% was of 45,200 cps with a range of between about 42,000 to 52,000 cps. The color was off white to slightly beige. The odor was characteristic to slightly fruity. The appearance was that of a moderately viscous cream with a smooth texture. The specific gravity was of 1.002+/−0.02 a stainless steel grease pychnometer at 25 degrees centigrade with a range of 0.982-1.022.

### Example 29

Formula 1B-R16-FF: Anti-aging Face Gel

| Ingredient (trademark) | Function(s) | Group # | % W/w |
|---|---|---|---|
| Water | Moisturizer | 13 | 79.530 |
| Glycerin | Hydrating agent, skin conditioning, skin protectant | 2 | 3 |
| Sodium carbomer (PNC-430) | Emulsion stabilizing agent, viscosity increasing | 4 | 0.8 |
| | agent | | |
| Ammonium Acryloyldimethyltaurate/VP | Viscosity increasing agent | 4 | 1.5 |
| copolymer (Aristoflex AVC) | | | |
| Squalane HQ (olive oil derived) | Hydrating agent, emollient | 5 | 4 |
| Tocopheryl Acetate (Vitamin E acetate) | Hydrating agent | 5 | 0.1 |
| Polysorbate-40 (Tween 40) | Emulsifying agent | 5 | 1.1 |
| Alpha bisabolol racemic (bisabolol) | Anti-irritation agent, anti-inflammatory and | 5 | 0.02 |
| | antibacterial agent | | |
| Dipalmitoyl hydroxyproline (sepilift DPHP) | Fibroblast relaxation (reduce expression wrinkles), | 5 | 1 |
| | anti MMP, anti-elastase, synthesis of TIMP2, | | |
| | stimulates lamins, anti-oxidant, anti-inflammatory | | |
| Glycosaminoglycans (MRTEX complex) | Anti-mmps; skin matrix integrity, anti-inflammatory | 1 | 2 |
| Hyaluronic Acid, water (hyasol BT 1%) | Hydrating agent | 1 | 2 |
| Ethylbisiminomethylguaiacol manganese | Anti-oxidant, oxygenation, DNA self-repair | 2 | 0.01 |
| chloride (Euk-134) | | | |
| Creatine (TegoCosmo c-100) | ATP stimulation | 1 | 0.01 |
| Propylene Glycol (humectant, solvent), | Hydrating agent | 1 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Sesamum indicum seed oil, triticum vulgare | Anti-inflammatory active, anti-irritation agent, | 1 | 0.03 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory active, hydrating agent | 1 | 0.02 |
| Alteromonas ferment extract, butylene glycol | Anti-irritation agent, langherhans cell protection, | 1 | 0.02 |
| (Abyssine 657) | skin matrix integrity | | |
| Imperata Cylindrica root extract, water, | Hydrating agent, osmosis protection | 1 | 3 |
| Glycerin, PEG-8, Carbomer (Moist 24) | | | |
| PEG-6 isostearate, hesperetin laurate | Anti-elastase, vaso-regulatory, anti-oxidant, anti- | 1 | 0.75 |
| (Flavagrum PEG) | inflammatory, anti-puffiness | | |
| 1,2-hexanediol, caprylyl glycol (Symdiol 68) | Hydrating agent, preservative | 1 | 1 |
| Water (and) glycerin (and) rumex (Tyrostat- | Inhibition of tyrosinase | 3 | 0.010 |
| 11) | | | |
| | | | 100 |

Manufacturing Process

1. In a stainless steel container, the ingredients of group 1 as identified in the Table above were mixed together with a Greaves™ homogenizer set at 5±1 or a Greaves 2™ homogenizer at 1800±200 rpm. The mixture was homogenized until a uniform mixture was obtained.

2. In a stainless steel container, Glycerin was added and the Ethylbisiminomethylguaiacol was sifted (ingredients of group 2). The mixture was agitated with the manual homogenizer for 5 minutes then added to the mixture of ingredients of group 1. The resulting mixture of the ingredients of groups 1 and 2 was agitated for a minimum of 20 minutes with a Greaves™ homogenizer set at 5±1 or a Greaves 2™ homogenizer at 1800±200 rpm. The sample was inspected visually to ensure it was free of undissolved brown particles.

3. In a small Lee™ tank, the water was heated to 75±5° C. Then, while agitating with the Greaves™ homogenizer set at 5±1 or the Greaves 2™ homogenizer at 1800±200 rpm, part of the Water (and) glycerin (and) rumex (Tyrostat-11) (group 3) was added. The mixture was then agitated for a minimum of 15 minutes or until a homogenous mixture was obtained.

4. While maintaining the agitation with the Greaves™ homogenizer set at 5±1 or the Greaves 2™ homogenizer at 1800±200 rpm and the temperature of the mixture at 80±2° C., ingredients of the group 4 were added as follows: the Sodium carbomer was slowly added to the mixture step 3. The mixture was then mixed well for a minimum of 15 minutes without exceeding 20 minutes and then the Ammonium acryloyldimet./VP copolymer was slowly added. The mixture was agitated until a homogenous mixture was obtained.

5. In a Coulter tank or in a stainless steel container, the ingredients of the group 5 were added as follows: the Polysorbate 40 and the Squalane HQ were added and mixed for 5 minutes. Then, Vitamin E, Alpha bisabolol racemic, and Dipalmitoyl hydroxyproline were added. The mixture was agitated with a Greaves™ homogenizer set at 5±1 or a Greaves 2™ homogenizer at 1800±200 rpm until a uniform mixture was obtained.

6. When the temperature of the oily phase (step 5) reached 80±2° C. and that of the aqueous phase (step 4) reaches 80±2° C., the mixture of step 5 was transferred into the mixture of step 4, while agitating. Then, the agitation was continued for a minimum of 15 minutes with the Greaves™ homogenizer set at 5±1 or the Greaves 2™ homogenizer at 1800±200 rpm.

7. The mixture of step 6 was cooled down to 48±2° C. while maintaining the agitation with the Greaves™ set at 3±1 or the Greaves 2™ set at 1400±200 rpm until a homogenous mixture was obtained.

8. The mixture of step 7 was slowly added to the mixture of step 2 while agitating with the Greaves™ homogenizer set at 5±1 or a Greaves 2™ homogenizer at 1800±200 rpm. Then, the agitation was maintained for a minimum of 10 minutes without exceeding 20 minutes or until a smooth and homogenous mixture was obtained. A sample of the mixture was inspected visually to ensure that it was free of undissolved particles.

9. The mixture was then cooled down to 25±2° C. A sample of the mixture was inspected a visually to ensure that it was free of undissolved particles.

### Specifications

The pH was of 6.1 with typical values between 5.0 and 6.5. The viscosity calculated on a Brookfield™ LVT Model DV I at 25 degrees Centigrade/1 minute, spindle #4 at 6 RPMs, dial reading at 45% was of 34,000 cps with a range of between about 30,000 to 45,000 cps. The color was off white. The odor was characteristic to slightly fruity. The appearance was that of a white translucent gel.

### Accelerated Stability Testing

The stability of the product was tested with an accelerated aging process. It was subjected to 4° C., 25° C. and 40° C. during 3 months. Measures and observations were taken at time 0, 1 month, 2 months and 3 months. The following parameters were measured: pH, viscosity, colour, organoleptic observations, organoleptic perception on the skin and appearance and signs of separation. Observations of the appearance were made following sharp variations of temperature and after freeze-thaw cycles.

The pH remained fairly stable at all temperatures with a slight but acceptable decrease at 40° C. Viscosity decreased slightly but no liquefaction or excessive thickening was observed and no viscosity change was observed upon application on the skin. The decrease was of about 3,000 cps per month at room temperature but stabilization at 8,000 cps was expected within about 6 months to two years.

The fragrance and the colour were stable. The preparation remained stable at all temperatures without separation or syneresis. The gel handled well sharp temperature changes. It could however only resist to one freeze-thaw cycle. With subsequent cycles, its texture was firmer and showed signs of syneresis. This gel was considered fairly stable with an expected life span of about 2 years.

### Example 30

Formula 1A-R16-FF: Anti-aging Face Cream (without emulsifying agent, with different stabilizing agent,
with different Ammonium Acryloyldimethyltaurate)

| Ingredient (trademark) | Function(s) | Group # | % W/w |
|---|---|---|---|
| Water | Moisturizer | 13 | 80.730 |
| Glycerin | Hydrating agent, skin conditioning, skin protectant | 3 | 3 |
| Potassium carbomer | Emulsion stabilizing agent, viscosity increasing | 5 | 1 |
| | agent | | |
| Ammonium | Viscosity increasing agent | 5 | 1.2 |
| Acryloyldimethyltaurate/Beheneth-25 | | | |
| Methacrylate Crosspolymer (Aristoflex | | | |
| HMB) | | | |
| Squalane HQ (olive oil derived) | Hydrating agent, emollient | 6 | 4 |
| Tocopheryl Acetate (Vitamin E acetate) | Hydrating agent | 6 | 0.1 |
| Glycerin, water, Centella asiatica extract, | Cellular renewal, anti-irritation | 2 | 0.1 |
| Carica papaya extract, Iris florentina | | | |
| extract (Botamix Regenerating) | | | |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Glycosaminoglycans (MRTEX complex) | Anti-mmps; skin matrix integrity, anti-inflammatory | 2 | 2 |
| Hyaluronic Acid, water (hyasol BT 1%) | Hydrating agent | 2 | 2 |
| Ethylbisiminomethylguaiacol manganese | Anti-oxidant, oxygenation, DNA self-repair | 2 | 0.01 |
| chloride (Euk-134) | | | |
| Creatine (TegoCosmo c-100) | ATP stimulation | 2 | 0.01 |
| Sesamum indicum seed oil, triticum vulgare | Anti-inflammatory active, anti-irritation agent, | 2 | 0.03 |
| germ oil, tocopheryl acetate, caprylic/capric | hydrating agent | | |
| succinic triglyceride (LNST PF) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Water, Glycerin, Hesperidin Methyl | Anti-elastase, vaso-regulatory, anti-oxidant, anti- | 2 | 0.75 |
| Chalcone, Steareth 20, Dipeptide-2, | inflammatory, anti-puffiness | | |
| Palmitoyl Tetrapeptide-7 (Eyeliss) | | | |
| 1,2-hexanediol, caprylyl glycol (Symdiol 68) | Hydrating agent, preservative | 2 | 1 |
| Water (and) glycerin (and) rumex (Tyrostat-11) | Inhibition of tyrosinase | 4 | 0.010 |
| | | | 100 |

PF: paraben free

Manufacturing Process

1. In a stainless steel container, the ingredients of group 1 as identified in the Table above were mixed together with a Greaves™ homogenizer set at 5±1 or a Greaves 2™ homogenizer at 1800±200 rpm. The mixture was homogenized until a uniform mixture was obtained.

2. In a stainless steel container, ingredients of group 2 were added as follows: Glycerin was added and the Ethylbisiminomethylguaiacol was sifted (ingredients of group 2). The mixture was agitated with the manual homogenizer for 5 minutes then added to the mixture of ingredients of group 1. The mixture of ingredients of groups 1 and 2 was agitated for a minimum of 10 minutes with a Greaves™ homogenizer set at 5±1 or a Greaves 2™ homogenizer at 1800±200 rpm. The sample was inspected visually to ensure that it was free of undissolved brown particles.

3. In a stainless steel container, part of the water and the Tyrostat-11 (group 3) were added and this mixture was heated to 75±5° C. Then, while agitating with the manual homogenizer. The mixture was homogenized for a minimum of 15 minutes or until a homogenous mixture was obtained.

4. While maintaining the agitation with the Greaves™ homogenizer set at 5±1 or the Greaves 2™ homogenizer at 1800±200 rpm and the temperature of the mixture at 80±2° C., ingredients of group 4 were added as follows: the potassium carbomer was slowly added to the mixture step 3. The mixture was then mixed well for a minimum of 5 minutes and then the Ammonium acryloyldimet./Beheneth-25 Methacrylate Crosspolymer was slowly added. The mixture was agitated until a homogenous mixture was obtained.

5. In a Coulter tank or in a stainless steel container, ingredients of group 5 were added as follows: the Squalane HQ, Vitamin E, Alpha bisabolol racemic, and Dipalmitoyl hydroxyproline. The mixture was heated to 80+/−2° C. while agitating with a Greaves™ homogenizer set at 5±1 or a Greaves 2™ homogenizer at 1800±200 rpm until a uniform mixture was obtained.

6. When the temperature of the oily phase (step 5) reached 80±2° C. and that of the aqueous phase (step 4) reaches 80±2° C., the mixture of step 5 was transferred into the mixture of step 4, while agitating. Then, the agitation was continued for a minimum of 15 minutes with the Greaves™ homogenizer set at 5±1 or the Greaves 2™ homogenizer at 1800±200 rpm.

7. The homogenizer was stopped and the mixture of step 6 was cooled down to 48±2° C.

8. The mixture of step 7 was slowly added to the mixture of step 2 while agitating with the Greaves™ homogenizer set at 5±1 or a Greaves 2™ homogenizer at 1800±200 rpm. Then, the agitation was maintained for a minimum of 10 minutes without exceeding 20 minutes or until a smooth and homogenous mixture was obtained. A sample of the mixture was inspected visually to ensure that it was free of undissolved particles. The mixture was then cooled down to 25±2° C. A sample of the mixture was inspected a visually to ensure that it was free of undissolved particles.

### Specifications

The pH was of 6.0 with typical values between 5.0 and 6.5. The viscosity calculated on a Brookfield™ LVT Model DV I at 25 degrees Centigrade/1 minute, spindle #4 at 6 RPMs, dial reading at 45% was of 44,000 cps with a range of between about 40,000 to 55,000 cps. The color was off white to slightly beige. The odor was characteristic to slightly fruity. The appearance was that of a moderately viscous cream with smooth texture.

### Accelerated Stability Testing

The stability of the product was tested with an accelerated aging process. It was subjected to 4° C., 25° C. and 40° C. during 3 months. Measures and observations were taken at time 0, 1 month, 2 months and 3 months. The following parameters were measured: pH, viscosity, colour, organoleptic observations, organoleptic perception on the skin and appearance and signs of separation. Observations of the appearance were made following sharp variations of temperature and after freeze-thaw cycles.

The pH remained stable at all temperatures with a slight but acceptable decrease at 40° C. Viscosity decreased slightly but no liquefaction or excessive thickening was observed and no viscosity change was observed upon application on the skin. The decrease was of about 1,000 cps per month at room temperature but stabilization at about 8,000 cps was expected within about 6 months to two years.

The fragrance was stable. The colour at the surface of the cream darkened and the browning rate correlated to the temperature. Light did not accelerate browning. It was estimated that the cream would become beige within one year. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered fairly stable with an expected life span of about 2 years.

### Example 31

Formula 2A-R16: Anti-Aging Face Serum (without emulsifying agent and with different stabilizing agent)

| Ingredient (trademark) | Function(s) | Group # | % W/w |
|---|---|---|---|
| Water | Moisturizer | 19 | 46.717 |
| Glycerin | Hydrating agent, skin conditioning, skin protectant | 3 | 3 |
| PEG-8/SMDI Copolymer (polyoprepolymer- | Controlled delivery system to avoid irritation and | 4 | 2 |
| 15) | inflammation | | |
| Carbomer (Synthalen L) | Emulsion stabilizing agent, viscosity increasing | 5 | 0.8 |
| | agent | | |
| Ammonium Acryloyldimethyltaurate/VP | Viscosity increasing agent | 5 | 1.7 |
| copolymer (Aristoflex AVC) | | | |
| Dimethicone, polysilicone-11 (gransil DMG-6) | Skin conditioning, skin protectant | 7 | 2.1 |
| Phenyl trimethicone, polysilicone 11 (Gransil | Detackifying agent | 7 | 2 |
| PM gel) | | | |
| Squalane HQ (olive oil derived) (squalane) | Hydrating agent, emollient | 6 | 4 |
| Tocopheryl Acetate (Vitamin E acetate) | Hydrating agent | 6 | 0.1 |
| Aluminum Benzoate, mica, Disodium | Surface modifier, UV light absorber, cosmetic | 6 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 6 | 3 |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 8 | 2.1 |
| GCM) | | | |
| Glycosaminoglycans (MDI complex PF | Mmps inhibitor; skin matrix integrity, anti- | 2 | 5 |
| paraben free) | inflammatory | | |
| Hyaluronic Acid, water (hyasol BT 1%) | Hydrating agent | 2 | 2 |
| Ethylbisiminomethylguaiacol manganese | Anti-oxidant, oxygenation, DNA self-repair | 3 | 0.01 |
| chloride (Euk-134) | | | |
| Retinol (Retinol 50c liquid + polysorbate 20) | Cellular renewal | 2 | 0.003 |
| Creatine (TegoCosmo c-100) | ATP stimulation | 2 | 0.01 |
| Glycerin, butylene glycol, water, carbomer | Collagen synthesis | 2 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 2 | 0.03 |
| germ oil, tocopheryl acetate, caprylic/capric | hydrating agent | | |
| succinic triglyceride (LNST PF (paraben | | | |
| free)) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Ceramide 3, 6 II, 1, Phytosphingosine, | Hydrating agent, Inhibition of tyrosinase activity | 2 | 5 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 2 | 1 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman 6) | Improves skin barrier function, anti-oxidant | 2 | 0.05 |
| Water, butylene glycol, dextran, palmitoyl | Anti-inflammatory | 2 | 2 |
| tripeptide-8 (Neutrazen) | | | |
| Ascophyllum nodosum extract, sorbitol, | Cellular renewal | 2 | 0.05 |
| water (Homeo age) | | | |
| Fucus serratus extract, glycerol (Homeo | Improves skin barrier function | 2 | 0.05 |
| shield) | | | |
| Ascophyllum nodosum extract, sorbitol, | Cellular renewal | 2 | 0.05 |
| water (Homeo soothe) | | | |
| Ubiquinone (coenzyme Q10) | Cellular renewal | 2 | 0.01 |
| Water, dextran, acetyl tetrapeptide-2 | Reinforcing the cutaneous immune defences and | 2 | 0.05 |
| (Thymulen 4BG) | cellular renewal | | |
| Water, sorbitol, ascophyllum nodosum | Skin Matrix integrity (anti-angiogenesis) | 2 | 2 |
| extract, asparagopsis armata extract | | | |
| (aldavine) | | | |
| Water, butylene glycol, Ahnfeltia Concinna | Hydrating agent, cellular renewal | 2 | 0.02 |
| Extract (APT) | | | |
| Hydrolyzed Rice Bran Protein, Glycine Soja | Anti-puffiness | 2 | 0.02 |
| protein, Oxido reductases (Regu-age) | | | |
| 1,2-hexanediol, caprylyl glycol (Symdiol 68) | Hydrating agent, preservative | 2 | 1 |
| Fragrance (Juniper Breeze #55472 | Produces or masks odours | 2 | 0.1500 |
| (Interome)) | | | |
| Water, acetyl octapeptide-3 (snap-8 solution C) | Cellular renewal | 1 | 2 |
| Sodium DNA from sturgeons (HDPR) | Inhibition of tyrosinase activity, hydrating agent, | 1 | 1 |
| | Cellular renewal, anti-aging, anti-wrinkle | | |
| Salix Alba bark extract (Carrubba willow | Cellular renewal | 1 | 5 |
| bark extract A9688) | | | |
| Glycerin, water, Centella asiatica extract, | Cellular renewal | 2 | 0.1 |
| Carica papaya extract, Iris florentina extract | | | |
| (Botamix Regenerating) | | | |
| Water (and) glycerin (and) rumex (Tyrostat-11) | Inhibition of tyrosinase | 2 | 0.010 |
| Water, Glycerin, Hesperidin Methyl | Anti-elastase, vaso-regulatory, anti-oxidant, anti- | 2 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil CQ) | Preservative | 7 | 0.5 |
| Dehydracetic Acid (Geogard 111 A) | Preservative | 9 | 0.15 |
| | | | 100 |

PF: paraben free

Manufacturing Process

1. In a stainless steel container, the ingredients of group 1 as identified in the Table above were mixed together with a manual homogenizer for about 10 minutes or until a uniform mixture was obtained. The mixture was allowed to stand until it was incorporated at a later step. The HDPR can be pre-hydrated separately by adding heat (<60° C.) and agitation in order to shorten this step.

2. In a coulter tank or a stainless steel container, while agitating using the Greaves™ Homogenizer set at 5±1 or the Greaves 2™ Homogenizer set at 1800±200 rpm, the ingredients of group 2 as identified the Table above were mixed together. The mixture was agitated until a uniform mixture was obtained.

3. In a stainless steel container, Glycerin was added and the Ethylbisiminomethylguaiacol was sprinkled (ingredients of group 3). The mixture was agitated with a manual homogenizer about 10 minutes or until a homogenous mixture was obtained.

4. The mixture of step 3 was transferred into the mixture of step 2 while continuing agitation for a minimum of 15 minutes. The sample was inspected visually to ensure that it was free of undissolved brown particles.

5. The mixture of step 4 was transferred into the mixture of step 1 while continuing agitation for a minimum of 15 minutes. (mixture of ingredients of groups 1, 2 and 3)

6. In a small Lee™ tank, water was heated to 75±5° C. Then, while continuing the agitation with a Greaves™ homogenizer set at 5±1 or a Greaves 2™ homogenizer set at 1800±200 rpm, the PEG-8/SMDI copolymer was added (ingredient of group 4). Agitating was continued for a minimum of 15 minutes or until a homogenous mixture was obtained.

7. While maintaining the temperature of the mixture of step 6 at 75±2° C. and agitating with a Greaves™ homogenizer set at 5±1 or a Greaves 2™ homogenizer at 1800±200 rpm, ingredients of group 5 were added as follows: the Carbomer (Synthalen L) was slowly added. The mixture was mixed well for a minimum of 10 minutes without exceeding 15 minutes then the Ammonium acryloyldimet/VP copolymer (Aristoflex AVC) was slowly added (ingredients of group 5). The mixture was mixed until a homogenous mixture was obtained. (mixture of ingredients of groups 4 and 5)

8. In a Coulter tank or in a stainless steel container, while agitating with the Leeson™ homogenizer set at 30±10 or the Leeson 2™ homogenizer set at 5±1 or the Baldor™ homogenizer set at 80±10, the ingredients of group 6 identified in the Table above were mixed and melted together at 80±2° C. Then using a manual homogenizer, the ingredients of group 7 identified in the Table above were added and the mixture was mixed until a uniform mixture was obtained. (mixture of ingredients of groups 6 and 7)

9. When the temperature of the oily phase (step 8) reached 80±2° C. and that of the aqueous phase (step 7) reached 75±2° C., the mixture of step 8 was transferred into the mixture of step 7, while agitating. Then, the agitation was continued for a minimum of 20 minutes with the Greaves™ homogenizer set at 5±1 or the Greaves 2™ homogenizer at 1800±200 rpm. (mixture of ingredients of groups 4-7)

10. The homogenization of the mixture of step 9 was continued with the Greaves™ homogenizer set at 4±1 or the Greaves 2™ homogenizer at 1600±200 rpm until a uniform mixture was obtained and then was cooled down to 48±2° C. (mixture of ingredients of groups 4-7)

11. Using the Greaves™ homogenizer set at 4±1 or the Greaves 2™ homogenizer set at 1600±200 rpm, the Cyclomethicone & polysilicone-11 (Gransil GCM) (ingredient of group 8) was added to the mixture of step 10. The mixture was agitated for a minimum of 10 minutes without exceeding 20 minutes or until a homogenous mixture was obtained. (mixture of ingredients of groups 4-8)

12. The mixture of step 11 was then cooled down to 38±2° C. while agitating with the Greaves™ homogenizer set at 4±1 or the Greaves 2™ set at 1600±200 rpm. (mixture of ingredients of groups 4-8)

13. In a stainless steel container, the water and the dehydracetic acid (Geogard 111 A) (ingredients of group 9) were poured and mixed with a spatula until a homogenous mixture was obtained. (mixture of ingredients of group 9).

14. The mixtures from steps 5 and 13 were transferred into the mixture of step 12 while agitating with the Greaves™ set at 5±1 or Greaves 2™ set at 1800±200 rpm. Then, agitation was continued for a minimum of 10 minutes without exceeding 20 minutes or until a smooth and homogenous mixture was obtained. The mixture was cooled down to 25±2° C. A sample of the mixture wa inspected visually to ensure thay it was free of brown or undissolved particles.

### Specifications

The pH was of 5.5 with typical values between 5.0 and 6.5. The viscosity calculated on a Brookfield™ LVT Model DV I at 25 degrees Centigrade/1 minute, spindle #4 at 6 RPMs, dial reading at 45% was of 41,000 cps with a range of between about 40,000 to 55,000 cps. The color was off white to slightly beige. The odor was characteristic to slightly fruity. The appearance was that of a moderately viscous cream with smooth texture.

### Accelerated Stability Testing

The stability of the product was tested with an accelerated aging process. It was subjected to 4° C., 25° C. and 40° C. during 3 months. Measures and observations were taken at time 0, 1 month, 2 months and 3 months. The following parameters were measured: pH, viscosity, colour, organoleptic observations, organoleptic perception on the skin and appearance and signs of separation. Observations of the appearance were made following sharp variations of temperature and after freeze-thaw cycles.

The pH remained stable at all temperatures with a slight but acceptable decrease at 40° C. Viscosity decreased slightly but no liquefaction or excessive thickening was observed and no viscosity change was observed upon application on the skin. The decrease was of about 1,000 cps per month at room temperature but stabilization at 8,000 cps was expected within about 6 months to two years.

The fragrance was stable although its intensity progressively weakened over time. The fragrance started to change at 40° C. at 3 months, which could be equated, to about 2 years at 25° C. The colour at the surface of the cream darkened and the browning rate correlated to the temperature. Light did not accelerate browning. It was estimated that the cream would become beige within one year. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered fairly stable with an expected life span of about 2 years.

### Example 32

Formula 3A-R16: Anti-Aging Face Serum (with different stabilizing agent and emulsifying agent 1)

| Ingredient (trademark) | Function(s) | Group # | % W/w |
|---|---|---|---|
| Water | Moisturizer | 19 | 45.217 |
| Glycerin | Hydrating agent, skin conditioning, skin protectant | 3 | 3 |
| PEG-8/SMDI Copolymer (polyoprepolymer- | Controlled delivery system to avoid irritation and | 4 | 2 |
| 15) | inflammation | | |
| Calcium potassium carbomer | Emulsion stabilizing agent, viscosity increasing | 5 | 0.8 |
| | agent | | |
| Ammonium Acryloyldimethyltaurate/VP | Viscosity increasing agent | 5 | 1.7 |
| copolymer (Aristoflex AVC) | | | |
| Dimethicone, polysilicone-11 (gransil DMG- | Skin conditioning, skin protectant | 7 | 2.1 |
| 6) | | | |
| Phenyl trimethicone, polysilicone 11 (Gransil | Detackifying agent | 7 | 2 |
| PM gel) | | | |
| Squalane HQ (olive oil derived) (squalane) | Hydrating agent, emollient | 6 | 4 |
| Tocopheryl Acetate (Vitamin E acetate) | Hydrating agent | 6 | 0.1 |
| Sorbitan monoleate (Tween 80) | Emulsifying agent | 6 | 1.5 |
| Aluminum Benzoate, mica, Disodium | Surface modifier, UV light absorber, cosmetic | 6 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 6 | 3 |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 8 | 2.1 |
| GCM) | | | |
| Glycosaminoglycans (MDI complex PF | Mmps inhibitor; skin matrix integrity, anti- | 2 | 5 |
| paraben free) | inflammatory | | |
| Hyaluronic Acid, water (hyasol BT 1%) | Hydrating agent | 2 | 2 |
| Ethylbisiminomethylguaiacol manganese | Anti-oxidant, oxygenation, DNA self-repair | 3 | 0.01 |
| chloride (Euk-134) | | | |
| Retinol (Retinol 50c liquid + polysorbate 20) | Cellular renewal | 2 | 0.003 |
| Creatine (TegoCosmo c-100) | ATP stimulation | 2 | 0.01 |
| Glycerin, butylene glycol, water, carbomer | Collagen synthesis | 2 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 2 | 0.03 |
| germ oil, tocopheryl acetate, caprylic/capric | hydrating agent | | |
| succinic triglyceride (LNST PF (without | | | |
| paraben)) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Ceramide 3, 6 II, 1, Phytosphingosine, | Hydrating agent, Inhibition of tyrosinase activity | 2 | 5 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 2 | 1 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman 6) | Improves skin barrier function, anti-oxidant | 2 | 0.05 |
| Water, butylene glycol, dextran, palmitoyl | Anti-inflammatory agent | 2 | 2 |
| tripeptide-8 (Neutrazen) | | | |
| Ascophyllum nodosum extract, sorbitol, | Cellular renewal | 2 | 0.05 |
| water (Homeo age) | | | |
| Fucus serratus extract, glycerol (Homeo | Improves skin barrier function | 2 | 0.05 |
| shield) | | | |
| Ascophyllum nodosum extract, sorbitol, | Cellular renewal | 2 | 0.05 |
| water (Homeo soothe) | | | |
| Ubiquinone (coenzyme Q10) | Cellular renewal | 2 | 0.01 |
| Water, dextran, acetyl tetrapeptide-2 | Reinforcing the cutaneous immune defences, | 2 | 0.05 |
| (Thymulen 4BG) | cellular renewal | | |
| Water, sorbitol, ascophyllum nodosum | Skin Matrix integrity (anti-angiogenesis) | 2 | 2 |
| extract, asparagopsis armata extract | | | |
| (aldavine) | | | |
| Water, butylene glycol, Ahnfeltia Concinna | Hydrating agent, cellular renewal | 2 | 0.02 |
| Extract (APT) | | | |
| Hydrolyzed Rice Bran Protein, Glycine Soja | Anti-puffiness | 2 | 0.02 |
| protein, Oxido reductases (Regu-age) | | | |
| 1,2-hexanediol, caprylyl glycol (Symdiol 68) | Hydrating agent, preservative | 2 | 1 |
| Fragrance (Juniper Breeze #55472 | Produces or masks odours | 2 | 0.1500 |
| (Interome)) | | | |
| Water, acetyl octapeptide-3 (snap-8 solution | Cellular renewal | 1 | 2 |
| C) | | | |
| Sodium DNA from sturgeons (HDPR) | Inhibition of tyrosinase activity, hydrating agent, | 1 | 1 |
| | Cellular renewal, anti-aging, anti-wrinkle | | |
| Salix Alba bark extract (Carrubba willow | Cellular renewal | 1 | 5 |
| bark extract A9688) | | | |
| Glycerin, water, Centella asiatica extract, | Cellular renewal | 2 | 0.1 |
| Carica papaya extract, Iris florentina extract | | | |
| (Botamix Regenerating) | | | |
| Water (and) glycerin (and) rumex (Tyrostat- | Inhibition of tyrosinase | 2 | 0.010 |
| 11) | | | |
| Water, Glycerin, Hesperidin Methyl | Anti-elastase, vaso-regulatory, anti-oxidant, anti- | 2 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil | Preservative | 7 | 0.5 |
| CQ) | | | |
| Dehydracetic Acid (Geogard 111 A) | Preservative | 9 | 0.15 |
| | | | 100 |

PF: paraben free

### Manufacturing Process

The composition was prepared by following the steps described in Example 31 above except that calcium potassium carbomer was added as emulsion stabilizing agent instead of carbomer.

### Specifications

The pH was of 5.4 with typical values between 5.0 and 6.5. The viscosity calculated on a Brookfield LVT Model DV I at 25 degrees Centigrade/1 minute, spindle #4 at 6 RPMs, dial reading at 45% was of 44,000 cps with a range of between about 40,000 to 55,000 cps. The color was off white to slightly beige. The odor was typical of the fragrance used. The appearance was that of a moderately viscous cream with smooth texture.

### Accelerated Stability Testing

Testing and results were as reported in Example 31 above.

### Example 33

Formula 3A-R16: Anti-Aging Face Serum (with different stabilizing agent and emulsifying agent 2)

| Ingredient (trademark) | Function(s) | Group # | % W/w |
|---|---|---|---|
| Water | Moisturizer | 19 | 45.217 |
| Glycerin | Hydrating agent, skin conditioning, skin protectant | 3 | 3 |
| PEG-8/SMDI Copolymer (polyoprepolymer- | Controlled delivery system to avoid irritation and | 4 | 2 |
| 15) | inflammation | | |
| Carbomer (Synthalen L) | Emulsion stabilizing agent, viscosity increasing | 5 | 0.8 |
| | agent | | |
| Ammonium Acryloyldimethyltaurate/VP | Viscosity increasing agent | 5 | 1.7 |
| copolymer (Aristoflex AVC) | | | |
| Dimethicone, polysilicone-11 (gransil DMG- | Skin conditioning, skin protectant | 7 | 2.1 |
| 6) | | | |
| Phenyl trimethicone, polysilicone 11 (Gransil | Detackifying agent | 7 | 2 |
| PM gel) | | | |
| Squalane HQ (olive oil derived) (squalane) | Hydrating agent, emollient | 6 | 4 |
| Tocopheryl Acetate (Vitamin E acetate) | Hydrating agent | 6 | 0.1 |
| PEG-20 Stearate (Lumulse 100-S) | Emulsifying agent | 6 | 1.5 |
| Aluminum Benzoate, mica, Disodium | Surface modifier, UV light absorber, cosmetic | 6 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 6 | 3 |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 8 | 2.1 |
| GCM) | | | |
| Glycosaminoglycans (MDI complex PF | Mmps inhibitor; skin matrix integrity, anti- | 2 | 5 |
| paraben free) | inflammatory | | |
| Hyaluronic Acid, water (hyasol BT 1%) | Hydrating agent | 2 | 2 |
| Ethylbisiminomethylguaiacol manganese | Anti-oxidant, oxygenation, DNA self-repair | 3 | 0.01 |
| chloride (Euk-134) | | | |
| Retinol (Retinol 50c liquid + polysorbate 20) | Cellular renewal | 2 | 0.003 |
| Creatine (TegoCosmo c-100) | ATP stimulation | 2 | 0.01 |
| Glycerin, butylene glycol, water, carbomer | Collagen synthesis | 2 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 2 | 0.03 |
| germ oil, tocopheryl acetate, caprylic/capric | hydrating agent | | |
| succinic triglyceride (LNST PF (without | | | |
| paraben)) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Ceramide 3, 6 II, 1, Phytosphingosine, | Hydrating agent, Inhibition of tyrosinase activity | 2 | 5 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 2 | 1 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman | Improves skin barrier function, anti-oxidant | 2 | 0.05 |
| 6) | | | |
| Water, butylene glycol, dextran, palmitoyl | Anti-inflammatory | 2 | 2 |
| tripeptide-8 (Neutrazen) | | | |
| Ascophyllum nodosum extract, sorbitol, | Cellular renewal | 2 | 0.05 |
| water (Homeo age) | | | |
| Fucus serratus extract, glycerol (Homeo | Improves skin barrier function | 2 | 0.05 |
| shield) | | | |
| Ascophyllum nodosum extract, sorbitol, | Cellular renewal | 2 | 0.05 |
| water (Homeo soothe) | | | |
| Ubiquinone (coenzyme Q10) | Cellular renewal | 2 | 0.01 |
| Water, dextran, acetyl tetrapeptide-2 | Reinforcing the cutaneous immune defences and | 2 | 0.05 |
| (Thymulen 4BG) | cellular renewal | | |
| Water, sorbitol, ascophyllum nodosum | Skin Matrix integrity (anti-angiogenesis) | 2 | 2 |
| extract, asparagopsis armata extract | | | |
| (aldavine) | | | |
| Water, butylene glycol, Ahnfeltia Concinna | Hydrating agent, cellular renewal | 2 | 0.02 |
| Extract (APT) | | | |
| Hydrolyzed Rice Bran Protein, Glycine Soja | Anti-puffiness | 2 | 0.02 |
| protein, Oxido reductases (Regu-age) | | | |
| 1,2-hexanediol, caprylyl glycol (Symdiol 68) | Hydrating agent, preservative | 2 | 1 |
| Fragrance (Juniper Breeze #55472 | Produces or masks odours | 2 | 0.1500 |
| (Interome)) | | | |
| Water, acetyl octapeptide-3 (snap-8 solution | Cellular renewal | 1 | 2 |
| C) | | | |
| Sodium DNA from sturgeons (HDPR) | Inhibition of tyrosinase activity, hydrating agent, | 1 | 1 |
| | Cellular renewal, anti-aging, anti-wrinkle | | |
| Salix Alba bark extract (Carrubba willow | Cellular renewal | 1 | 5 |
| bark extract A9688) | | | |
| Glycerin, water, Centella asiatica extract, | Cellular renewal, anti-irritation | 2 | 0.1 |
| Carica papaya extract, Iris florentina extract | | | |
| (Botamix Regenerating) | | | |
| Water (and) glycerin (and) rumex (Tyrostat- | Inhibition of tyrosinase | 2 | 0.010 |
| 11) | | | |
| Water, Glycerin, Hesperidin Methyl | Anti-elastase, vaso-regulatory, anti-oxidant, anti- | 2 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil | Preservative | 7 | 0.5 |
| CQ) | | | |
| Dehydracetic Acid (Geogard 111 A) | Preservative | 9 | 0.15 |
| | | | 100 |

PF: paraben free

### Manufacturing Process

The composition was prepared by following the steps described in Example 31 above.

### Specifications

The pH was of 5.5 with typical values between 5.0 and 6.5. The viscosity calculated on a Brookfield LVT Model DV I at 25 degrees Centigrade/1 minute, spindle #4 at 6 RPMs, dial reading at 45% was of 47,000 cps with a range of between about 40,000 to 55,000 cps. The color was off white to slightly beige. The odor was typical of the fragrance used. The appearance was that of a moderately viscous cream with smooth texture.

### Accelerated Stability Testing

Testing and results were as reported in Example 31 above.

### Example 34

Formula 4C-R16: Anti-Aging Face Serum (with different stabilizing agent and emulsifying agent)

| Ingredient (trademark) | Function(s) | Group # | % W/w |
|---|---|---|---|
| Water | Moisturizer | 19 | 45.117 |
| Glycerin | Hydrating agent, skin conditioning, skin protectant | 3 | 3 |
| PEG-8/SMDI Copolymer (polyoprepolymer- | Controlled delivery system to avoid irritation and | 4 | 2 |
| 15) | inflammation | | |
| Xanthan Gum (Keltrol HP) | Emulsion stabilizing agent, viscosity increasing | 5 | 1.2 |
| | agent | | |
| Ammonium Acryloyldimethyltaurate/VP | Viscosity increasing agent | 5 | 1.7 |
| copolymer (Aristoflex AVC) | | | |
| Dimethicone, polysilicone-11 (gransil DMG- | Skin conditioning, skin protectant | 7 | 2.1 |
| 6) | | | |
| Phenyl trimethicone, polysilicone 11 (Gransil | Detackifying agent | 7 | 2 |
| PM gel) | | | |
| Squalane HQ (olive oil derived) (squalane) | Hydrating agent, emollient | 6 | 4 |
| Tocopheryl Acetate (Vitamin E acetate) | Hydrating agent | 6 | 0.1 |
| Sorbitan monoleate (Tween 80) | Emulsifying agent | 6 | 1.2 |
| Aluminum Benzoate, mica, Disodium | Surface modifier, UV light absorber, cosmetic | 6 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 6 | 3 |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 8 | 2.1 |
| GCM) | | | |
| Glycosaminoglycans (MDI complex PF | Mmps inhibitor; skin matrix integrity, anti- | 2 | 5 |
| paraben free) | inflammatory | | |
| Hyaluronic Acid, water (hyasol BT 1%) | Hydrating agent | 2 | 2 |
| Ethylbisiminomethylguaiacol manganese | Anti-oxidant, oxygenation, DNA self-repair | 3 | 0.01 |
| chloride (Euk-134) | | | |
| Retinol (Retinol 50c liquid + polysorbate 20) | Cellular renewal | 2 | 0.003 |
| Creatine (TegoCosmo c-100) | ATP stimulation | 2 | 0.01 |
| Glycerin, butylene glycol, water, carbomer | Collagen synthesis | 2 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 2 | 0.03 |
| germ oil, tocopheryl acetate, caprylic/capric | hydrating agent | | |
| succinic triglyceride (LNST PF (without | | | |
| paraben)) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Ceramide 3, 6 II, 1, Phytosphingosine, | Hydrating agent, Inhibition of tyrosinase activity | 2 | 5 |
| Cholesterol, Sodium Lauroyl Lactylate, | | | |
| Carbomer, xanthan gum (sk-influx) | | | |
| Water, pseudoalteromonas ferment extract, | Skin Matrix integrity (anti-angiogenesis) | 2 | 1 |
| hydrolyzed wheat protein, hydrolized soy | | | |
| protein, tripeptide-10, citrulline, tripeptide-1, | | | |
| lecithin, xanthan gum, carbomer, | | | |
| triethanolamine (Trylagen PCB) | | | |
| Dimethylmethoxy Chromanol (Lipochroman | Improves skin barrier function, anti-oxidant | 2 | 0.05 |
| 6) | | | |
| Water, butylene glycol, dextran, palmitoyl | Anti-inflammatory | 2 | 2 |
| tripeptide-8 (Neutrazen) | | | |
| Ascophyllum nodosum extract, sorbitol, | Cellular renewal | 2 | 0.05 |
| water (Homeo age) | | | |
| Fucus serratus extract, glycerol (Homeo | Improves skin barrier function | 2 | 0.05 |
| shield) | | | |
| Ascophyllum nodosum extract, sorbitol, | Cellular renewal | 2 | 0.05 |
| water (Homeo soothe) | | | |
| Ubiquinone (coenzyme Q10) | Cellular renewal | 2 | 0.01 |
| Water, dextran, acetyl tetrapeptide-2 | Reinforcing the cutaneous immune defences, | 2 | 0.05 |
| (Thymulen 4BG) | cellular renewal | | |
| Water, sorbitol, ascophyllum nodosum | Skin Matrix integrity (anti-angiogenesis) | 2 | 2 |
| extract, asparagopsis armata extract | | | |
| (aldavine) | | | |
| Water, butylene glycol, Ahnfeltia Concinna | Hydrating agent, cellular renewal | 2 | 0.02 |
| Extract (APT) | | | |
| Hydrolyzed Rice Bran Protein, Glycine Soja | Anti-puffiness | 2 | 0.02 |
| protein, Oxido reductases (Regu-age) | | | |
| 1,2-hexanediol, caprylyl glycol (Symdiol 68) | Hydrating agent, preservative | 2 | 1 |
| Fragrance (Juniper Breeze #55472 | Produces or masks odours | 2 | 0.1500 |
| (Interome)) | | | |
| Water, acetyl octapeptide-3 (snap-8 solution | Cellular renewal | 1 | 2 |
| C) | | | |
| Sodium DNA from sturgeons (HDPR) | Inhibition of tyrosinase activity, hydrating agent, | 1 | 1 |
| | Cellular renewal, anti-aging, anti-wrinkle | | |
| Salix Alba bark extract (Carrubba willow | Cellular renewal | 1 | 5 |
| bark extract A9688) | | | |
| Glycerin, water, Centella asiatica extract, | Cellular renewal | 2 | 0.1 |
| Carica papaya extract, Iris florentina extract | | | |
| (Botamix Regenerating) | | | |
| Water (and) glycerin (and) rumex (Tyrostat-11) | Inhibition of tyrosinase | 2 | 0.010 |
| Water, Glycerin, Hesperidin Methyl | Anti-elastase, vaso-regulatory, anti-oxidant, anti- | 2 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil CQ) | Preservative | 7 | 0.5 |
| Dehydracetic Acid (Geogard 111 A) | Preservative | 9 | 0.15 |
| | | | 100 |

PF: paraben free

### Manufacturing Process

The composition was prepared by following the steps described in Example 31 above except xanthan gum was added as emulsion stabilizer instead of the carbomer.

### Specifications

The pH was of 5.6 with typical values between 5.0 and 6.5. The viscosity calculated on a Brookfield LVT Model DV I at 25 degrees Centigrade/1 minute, spindle #4 at 6 RPMs, dial reading at 45% was of 48,000 cps with a range of between about 40,000 to 55,000 cps. The color was off white to slightly beige. The odor was typical of the fragrance used. The appearance was that of a moderately viscous cream with smooth texture.

### Accelerated Stability Testing

Testing and results were as reported in Example 31 above.

### Example 35

Formula 7A-R16: Anti-Cellulite Gel

| Ingredient (trademark) | Function(s) | Group # | % W/w |
|---|---|---|---|
| Water | Moisturizer | 13 | 76.130 |
| Glycerin | Hydrating agent, skin conditioning, skin protectant | 2 | 3 |
| Sodium carbomer (PNC-430 (3V Inc.)) | Emulsion stabilizing agent, viscosity increasing | 3 | 0.9 |
| | agent | | |
| Ammonium Acryloyldimethyltaurate/VP | Viscosity increasing agent | 3 | 1.7 |
| copolymer (Aristoflex AVC) | | | |
| Squalane HQ (olive oil derived) | Hydrating agent, emollient | 4 | 4 |
| Tocopheryl Acetate (Vitamin E acetate) | Hydrating agent | 4 | 0.1 |
| Polysorbate-40 (Tween 40) | Emulsifying agent | 4 | 1.2 |
| Alpha Bisabolol racemic (bisabolol) | Anti-irritation agent, anti-inflammatory and | 4 | 0.02 |
| | antibacterial agent | | |
| Dipalmitoyl hydroxyproline (sepilift DPHP) | Fibroblast relaxation (reduce expression wrinkles), | 4 | 1 |
| | anti MMP, anti-elastase, synthesis of TIMP2, | | |
| | stimulates lamins, anti-oxidant, anti-inflammatory | | |
| Glycosaminoglycans (MRTEX complex) | Anti-mmps; skin matrix integrity, anti-inflammatory | 1 | 2 |
| Hyaluronic Acid, water (hyasol BT 1%) | Hydrating agent | 1 | 2 |
| Ethylbisiminomethylguaiacol manganese | Anti-oxidant, oxygenation, DNA self-repair | 2 | 0.01 |
| chloride (Euk-134) | | | |
| Creatine (TegoCosmo c-100) | ATP stimulation | 1 | 0.01 |
| Propylene Glycol (humectant, solvent), | Hydrating agent | 1 | 0.1 |
| Commiphora Myrrha Extract, Equisetum | | | |
| Arvense Extract, Triticum Vulgare Germ | | | |
| extract, Humulus lupulus Extract (330- | | | |
| Regederme HS) | | | |
| Sesamum indicum seed oil, triticum vulgare | Anti-inflammatory agent, anti-irritation agent, | 1 | 0.03 |
| germ oil, tocopheryl acetate, caprylic/capric | hydrating agent | | |
| succinic triglyceride (LNST 98) | | | |
| Dipotassium Glycyrrhizate (Net DG) | Anti-inflammatory agent, hydrating agent | 1 | 0.02 |
| Alteromonas ferment extract, butylene glycol | Anti-irritation agent, langherhans cell protection, | 1 | 0.02 |
| (Abyssine 657) | skin matrix integrity | | |
| Imperata Cylindrica root extract, water, | Hydrating agent, osmosis protection | 1 | 3 |
| Glycerin, PEG-8, Carbomer (Moist 24) | | | |
| PEG-6 isostearate, hesperetin laurate | Anti-elastase, vaso-regulatory, anti-oxidant, anti- | 1 | 0.75 |
| (Flavagrum PEG) | inflammatory, anti-puffiness | | |
| Glycerin/Aqua/Coco-Glucoside/Caprylyl | anti-cellulite activity | 1 | 0.250 |
| Glycol/Alcohol/Glaucine (Bodylift) | | | |
| Hydrolyzed Celosia Cristata Flower/Seed | anti-cellulite activity | 1 | 1.000 |
| Extract and Hydrolized Prunella Vulgaris | | | |
| Extract (BIOSCULPTINE) | | | |
| Butylene glycol, water, nelumbo nucifera leaf | anti-cellulite activity | 1 | 1.000 |
| extract (PRO-SVELTYL) | | | |
| Water, propylene glycol, citrus aurantium | anti-cellulite activity | 1 | 0.250 |
| amara (bitter orange) flower extract | | | |
| (REMODULINE) | | | |
| Water, butylene glycol, Peumus boldus leaf | anti-cellulite activity | 1 | 0.250 |
| extract (SLIMACTIVE) | | | |
| Cecrpia obtusa extract (SLIM FIT) | anti-cellulite activity | 1 | 0.250 |
| 1,2-hexanediol, caprylyl glycol (Symdiol 68) | Hydrating agent, preservative | 1 | 1 |
| Water (and) glycerin (and) rumex (Tyrostat-11) | Inhibition of tyrosinase | 1 | 0.010 |
| | | | 100 |

Manufacturing Process

1. In a coulter tank or a stainless steel container, while agitating using the Greaves™ Homogenizer set at 5±1 or the Greaves 2™ Homogenizer set at 1800±200 rpm, the ingredients of group 1 as identified the Table above were mixed together. The mixture was agitated until a uniform mixture was obtained.

2. In a stainless steel container, ingredients of group 2 were added as follows: Glycerin was added and the Ethylbisiminomethylguaiacol was sprinkled. The mixture was agitated with a manual homogenizer about 10 minutes or until a homogenous mixture was obtained.

3. The mixture of step 2 was transferred into the mixture of step 1 while continuing agitation for a minimum of 15 minutes. The sample was inspected visually to ensure that it was free of undissolved brown particles. (mixture of ingredients of groups 1 and 2)

4. In a small Lee™ tank, water was heated to 75±5° C. Then, while continuing the agitation with a Greaves™ homogenizer set at 5±1 or a Greaves 2™ homogenizer set at 1800±200 rpm, ingredients of group 3 were added as follows: the Sodium carbomer was slowly added. The mixture was mixed well for a minimum of 10 minutes without exceeding 15 minutes then the Ammonium acryloyldimet/VP copolymer (Aristoflex AVC) was slowly added. The mixture was mixed until a homogenous mixture was obtained. (mixture of ingredients of group 3)

5. In a Coulter tank or in a stainless steel container, while agitating with the Leeson™ homogenizer set at 30±10 or the Leeson 2™ homogenizer set at 5±1 or the Baldor™ homogenizer set at 80±10, the ingredients of group 4 identified in the Table above were mixed and melted together at 80±2° C. Then using a manual homogenizer, the mixture was mixed until a uniform mixture was obtained. (mixture of ingredients of group 4)

6. When the temperature of the oily phase (step 5) reached 80±2° C. and that of the aqueous phase (step 4) reached 75±2° C., the mixture of step 5 was transferred into the mixture of step 4, while agitating. Then, the agitation was continued for a minimum of 10 minutes with the Greaves™ homogenizer set at 5±1 or the Greaves 2™ homogenizer at 1800±200 rpm. (mixture of ingredients of groups 3-4)

7. The mixture of step 6 was agitated until a uniform gel was obtained and cooled down to 38±2° C. while agitating with the Greaves™ homogenizer set at 4±1 or the Greaves 2™ homogenizer set at 1600±200 rpm. (mixture of ingredients of groups 3-4)

8. The mixtures from step 3 was slowly transferred into the mixture of step 7 while agitating with the Greaves™ homogenizer set at 5±1 or Greaves 2™ homogenizer set at 1800±200 rpm. Then, agitation was continued for a minimum of 5 minutes without exceeding 10 minutes or until a smooth and homogenous mixture was obtained. The mixture was then cooled down to 25±2° C. A sample of the mixture wa inspected visually to ensure thay it was free of brown or undissolved particles.

### Specifications

The pH was of 5.8 with typical values between 5.0 and 6.5. The viscosity calculated on a Brookfield LVT Model DV I at 25 degrees Centigrade/1 minute, spindle #4 at 6 RPMs, dial reading at 45% was of 44,000 cps with a range of between about 40,000 to 55,000 cps. The color was slightly yellow. The odor was characteristic to slightly fruity. The appearance was that of a rather opaque gel.

### Accelerated Stability Testing

The stability of the product was tested with an accelerated aging process. It was subjected to 4° C., 25° C. and 40° C. during 3 months. Measures and observations were taken at time 0, 1 month, 2 months and 3 months. The following parameters were measured: pH, viscosity, colour, organoleptic observations, organoleptic perception on the skin and appearance and signs of separation. Observations of the appearance were made following sharp variations of temperature and after freeze-thaw cycles.

The pH remained fairly stable at all temperatures with a slight but acceptable decrease at 40° C. Viscosity decreased but no liquefaction or excessive thickening was observed and no viscosity change was observed upon application on the skin. The decrease was of about 2,000 cps per month at room temperature but stabilization at 8000 cps was expected within about 6 months to two years.

The fragrance was stable. The colour of the gel stayed quite the same. The gel remained stable at all temperatures without separation or syneresis. The gel handled well sharp temperature changes. It could however only resist to one freeze-thaw cycle. With subsequent cycles, its texture was firmer and showed signs of syneresis. This gel was considered fairly stable with an expected life span of about 2 years.

### Example 36

Formula R16-ASV: Rouge minime - Anti-Redness Integral Serum

| Ingredient (trademark) | Function(s) | Group # | % W/w |
|---|---|---|---|
| | | 14 | |
| Water | Moisturizer | 11 | 43.797 |
| Glycerin | Hydrating agent, skin conditioning, skin protectant | 4 | 3 |
| Disodium EDTA (Dissolvine Na2 (Akzo)) | Chelating agent | 4 | 0.1000 |
| PEG-8/SMDI Copolymer (polyoprepolymer- | Controlled delivery system to avoid irritation and | 4 | 2 |
| 15) | inflammation | | |
| Sodium Carbomer (PNC-430 (3V Inc.)) | Emulsion stabilizing agent, viscosity increasing | 5 | 0.8 |
| | agent | | |
| Ammonium Acryloyldimethyltaurate/VP | Viscosity increasing agent | 5 | 0.750 |
| copolymer (Aristoflex AVC) | | | |
| Dimethicone, polysilicone-11 (gransil DMG- | Skin conditioning, skin protectant | 9 | 2.1 |
| 6) | | | |
| Phenyl trimethicone, polysilicone 11 (Gransil | Detackifying agent | 9 | 1.5 |
| PM gel) | | | |
| Squalane HQ (olive oil derived) (squalane) | Hydrating agent, emollient | 6 | 4 |
| Tocopheryl Acetate (Vitamin E acetate) | Hydrating agent | 8 | 0.1 |
| Polysorbate 40 (Tween 40) | Emulsifying agent | 6 | 1.2 |
| Aluminum Benzoate, mica, Disodium | Surface modifier, UV light absorber, cosmetic | 8 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 6 | 3 |
| Alpha Bisabolol racemic (bisabolol) | Anti-irritation agent, anti-inflammatory and | 8 | 0.02 |
| | antibacterial agent | | |
| Dipalmitoyl hydroxyproline (Sepilift DPHP) | Fibroblast relaxation (reduce expression wrinkles), | 8 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 10 | 2.1 |
| GCM) | | | |
| Glycosaminoglycans (MDI complex PF | Mmps inhibitor; skin matrix integrity, anti- | 2 | 5 |
| paraben free) | inflammatory | | |
| Hyaluronic Acid, water (hyasol BT 1%) | Hydrating agent | 2 | 2 |
| Ethylbisiminomethylguaiacol manganese | Anti-oxidant, oxygenation, DNA self-repair | 7 | 0.01 |
| chloride (Euk-134) | | | |
| Cyclomethicone (Dow Corning 345 Fluid | Detackifying agent | 2 | 1.0000 |
| (Dow Corning)) | | | |
| Retinol (Retinol 50c liquid + polysorbate 20) | Cellular renewal | 2 | 0.003 |
| Creatine (TegoCosmo c-100) | ATP stimulation | 2 | 0.01 |
| Glycerin, butylene glycol, water, carbomer | Collagen synthesis | 2 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 2 | 0.03 |
| germ oil, tocopheryl acetate, caprylic/capric | Hydrating agent | | |
| succinic triglyceride (LNST PF (without | | | |
| paraben)) | | | |
| Dipotassium Glycyrrhizate (Net DG) | Anti-inflammatory agent, hydrating agent | 2 | 0.02 |
| Alteromonas ferment extract, butylene glycol | Anti-irritation agent, langherhans cell protection, | 2 | 0.02 |
| (Abyssine PF) | skin matrix integrity | | |
| Imperata Cylindrica root extract, water, | Hydrating agent, osmosis protection | 2 | 3 |
| Glycerin, PEG-8, Carbomer (Moist 24) | | | |
| Ceramide 3, 6 II, 1, Phytosphingosine, | Hydrating agent, Inhibition of tyrosinase activity | 2 | 5 |
| Cholesterol, Sodium Lauroyl Lactylate, | | | |
| Carbomer, xanthan gum (sk-influx) | | | |
| Dimethylmethoxy Chromanol (Lipochroman | Improves skin barrier function, anti-oxidant | 2 | 0.05 |
| 6) | | | |
| Water, butylene glycol, dextran, palmitoyl | Anti-inflammatory | 2 | 2 |
| tripeptide-8 (Neutrazen) | | | |
| Ascophyllum nodosum extract, sorbitol, | Cellular renewal | 2 | 0.05 |
| water (Homeo age) | | | |
| Fucus serratus extract, glycerol (Homeo | Improves skin barrier function | 2 | 0.05 |
| shield) | | | |
| Ascophyllum nodosum extract, sorbitol, | Cellular renewal | 2 | 0.05 |
| water (Homeo soothe) | | | |
| Ubiquinone (coenzyme Q10) | Cellular renewal | 2 | 0.01 |
| Water, dextran, acetyl tetrapeptide-2 | Reinforcing the cutaneous immune defences and | 2 | 0.05 |
| (Thymulen 4bg) | cellular renewal | | |
| Esculoside: Pure molecule from Bark | Venotonic, anti-inflammatory, anti-oxdant | 2 | 1 |
| Horse Chestnut | | | |
| Water, sorbitol, ascophyllum nodosum | Skin Matrix integrity (anti-angiogenesis) | 2 | 2 |
| extract, asparagopsis armata extract | | | |
| (Aldavine) | | | |
| Water, butylene glycol, Ahnfeltia Concinna | Hydrating agent, cellular renewal | 2 | 0.02 |
| Extract (APT) | | | |
| Hydrolyzed Rice Bran Protein, Glycine Soja | Anti-puffiness | 2 | 0.02 |
| protein, Oxido reductases (Regu-age) | | | |
| Fragrance (Juniper Breeze #55472 | Produces or masks odours | 2 | 0.1500 |
| (Interome)) | | | |
| Water, acetyl octapeptide-3 (snap-8 solution C) | Cellular renewal | 1 | 2 |
| Sodium DNA from sturgeons (HDPR) | Inhibition of tyrosinase activity, hydrating agent, | 1 | 1 |
| | Cellular renewal, anti-aging, anti-wrinkle | | |
| Salix Alba bark extract (Carrubba willow | Cellular renewal | 1 | 5 |
| bark extract A9688) | | | |
| Glycerin, water, Centella asiatica extract, | Celllular renewal | 2 | 0.1 |
| Carica papaya extract, Iris florentina extract | | | |
| (Botamix Regenerating) | | | |
| Water (and) glycerin (and) rumex (Tyrostat-11) | Inhibition of tyrosinase | 2 | 0.010 |
| Water, Glycerin, Hesperidin Methyl | Anti-elastase, vaso-regulatory, anti-oxidant, anti- | 2 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil CQ) | Preservative | 10 | 0.5 |
| Titanium Dioxide (and) Iron Oxides | Photochromic pigments | 7 | 2.0 |
| (Photolite PK-S) | | | |
| Titanium dioxide | sunscreen | 7 | 1.0 |
| Iron oxide yellow C33-130 4CO882 | colouring agent | 7 | 0.07 |
| Dehydracetic acid (Geogard 111 A) | Preservative | 11 | 0.15 |
| | | | 100 |

PF: paraben free

### Manufacturing Process

1. In a stainless steel container, the ingredients of group 1 as identified in the Table above were mixed together with a manual homogenizer for about 10 minutes or until a uniform mixture was obtained. The mixture was allowed to stand until it was incorporated at a later step. (mixture of ingredients of group 1)

2. In a coulter tank or a stainless steel container, while agitating using the Greaves™ Homogenizer set at 5±1 or the Greaves 2™ Homogenizer set at 1800±200 rpm, the ingredients of group 2 as identified in the Table above were mixed together. The mixture was agitated until a uniform mixture was obtained. (mixture of ingredients of group 2)

3. In a stainless steel container, Glycerin was added and the Ethylbisiminomethylguaiacol was sprinkled (ingredients of group 3). The mixture was agitated with a manual homogenizer about 5 minutes or until a homogenous mixture was obtained. This mixture was transferred into the mixture of step 2 (mixture of ingredients of groups 2-3)

4. The mixture of group 1 was transferred into the mixture of group 3 while continuing agitation using the Greaves™ Homogenizer set at 5±1 or the Greaves 2™ Homogenizer set at 1800±200 rpm for a minimum of 20 minutes. The sample was inspected visually to ensure that it was free of undissolved brown particles and of pigments. (mixture of ingredients of groups 1-3)

5. In a small Lee™ tank, water was heated to 75±5° C. Then, while continuing the agitation with a Greaves™ homogenizer set at 5±1 or a Greaves 2™ homogenizer set at 1800±200 rpm, Glycerin and Disodium EDTA were added. PEG-8/SDMI copolymer was then added (ingredients of group 4). The mixture was mixed for a minimum of 15 minutes or until a homogenous mixture was obtained. (mixture of ingredients of group 4)

6. Then, while continuing the agitation of the mixture of step 5 with a Greaves™ homogenizer set at 5±1 or a Greaves 2™ homogenizer set at 1800±200 rpm and maintaining the temperature of the mixture at 80+/−C, the sodium carbomer was slowly added. The mixture was mixed well for a minimum of 15 minutes without exceeding 20 minutes and then the Ammonium acryloyldimet/VP copolymer (Aristoflex AVC) was slowly added (ingredients of group 5). The mixture was mixed until a homogenous mixture was obtained. (mixture of ingredients of groups 4-5)

7. In a Coulter™ tank or in a stainless steel container, the ingredients of group 6 identified in the Table above were mixed together for 5 minutes. The ingredients of group 7 identified in the Table above were then added. The mixture was then agitated for 10 minutes with the Greaves™ homogenizer set at 5±1 or a Greaves 2™ homogenizer set at 1800±200 rpm. The ingredients of group 8 were then added. The mixture was heated together at 80±2° C., and while continuing the agitation, the ingredients of group 9 identified in the Table above were added and the mixture was mixed until a uniform mixture was obtained. (mixture of ingredients of groups 6-9).

8. When the temperature of the oily phase (step 7) reached 80±2° C. and that of the aqueous phase (step 6) reached 75±2° C., the mixture of step 7 was transferred into the mixture of step 6, while agitating. Then, the agitation was continued for a minimum of 15 minutes with the Greaves™ homogenizer set at 5±1 or the Greaves 2™ homogenizer at 1800±200 rpm. (mixture of ingredients of groups 4-9)

9. The mixture of step 9 was cooled down to 48±2° C. while agitating with the Greaves™ at 3±1 or the Greaves 2™ at 1400±200 rpm. (mixture of ingredients of groups 4-9)

10. Using the homogenizer, the Cyclomethicone & polysilicone-11 and the Polyaminopropyl biguanide were added (ingredients of group 10) to the mixture of step 9. The mixture was then mixed with the Greaves™ homogenizer at 5±1 or Greaves 2™ homogenizer at 1800±200 rpm, for a minimum de 10 minutes or until a homogeneous mixture was obtained. (mixtures of ingredients of groups 4-10)

11. The mixture of step 10 was cooled down to 38±2° C.

12. The mixture of step 4 was slowly transferred into the mixture of step 11 while agitating with the Greaves™ homogenizer set at 5±1 or Greaves 2™ homogenizer set at 1800±200 rpm. Then, agitation was continued for a minimum of 10 minutes without exceeding 20 minutes or until a smooth and homogenous mixture was obtained. (mixture of ingredients of groups 1-10)

13. In a stainless steel container, the water and Geogard 111 A were added (ingredients of group 11). The mixture was agitated with a spatula until a homogeneous mixture was obtained. (ingredients of group 11)

14. The mixture of step 13 was slowly transferred into the mixture of step 12 while agitating with the Greaves™ set at 5±1 or Greaves 2™ set at 1800±200 rpm. The mixture was cooled down to 25±2° C. A sample of the mixture was inspected visually to ensure that it was free of brown undissolved particles and did not contain any pigments.

### Specifications

The pH was of 5.7 with typical values between 5.0 and 6.5. The viscosity calculated on a Brookfield LVT Model DV I at 25 degrees Centigrade/1 minute, spindle #4 at 6 RPMs, dial reading at 45% was of 50,000 cps with a range of between about 40,000 to 55,000 cps. The color was light beige with a pinkish tone. The odor was typical of the fragrance used. The appearance was that of a smooth homogeneous cream.

### Accelerated Stability Testing

The stability of the product was tested with an accelerated aging process. It was subjected to 4° C., 25° C. and 40° C. during 3 months. Measures and observations were taken at time 0, 1 month, 2 months and 3 months. The following parameters were measures: pH, viscosity, colour, organoleptic observations, organoleptic perception on the skin and appearance and signs of separation. Observations of the appearance were made following sharp variations of temperature and after freeze-thaw cycles.

The pH remained stable at all temperatures. Viscosity was stable at 4° C. and 25° C. but, at 40° C., decreased sharply by 15,000 cps down to 34,400 cps after one month. However, no liquefaction or excessive thickening was observed and no viscosity change was observed upon application on the skin. The viscosity is expected to stabilized at around 8,000 cps within two years.

The fragrance was stable although its intensity progressively weakened over time. The fragrance started to change at 40° C. at 3 months, which could be equated, to about 2 years at 25° C. The colour of the cream tended to vary slightly from batch to batch but was rather stable upon time. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered fairly stable with an expected life span of about 2 years.

### Example 37

REGEN16 + Whitening Formulation

| Ingredient (trademark) | Function(s) | Group # | % W/w |
|---|---|---|---|
| Water | Moisturizer | 10 | 45.217 |
| Glycerin | Hydrating agent, skin conditioning, skin protectant | 2 | 3 |
| PEG-8/SMDI Copolymer (polyoprepolymer- | Controlled delivery system to avoid irritation and | 3 | 2 |
| 15) | inflammation | | |
| Sodium Carbomer (PNC-430 (3V Inc.)) | Emulsion stabilizing agent, viscosity increasing | 4 | 0.8 |
| | agent | | |
| Ammonium Acryloyldimethyltaurate/VP | Viscosity increasing agent | 4 | 1.7 |
| copolymer (Aristoflex AVC) | | | |
| Dimethicone, polysilicone-11 (gransil DMG- | Skin conditioning, skin protectant | 7 | 2.1 |
| 6) | | | |
| Phenyl trimethicone, polysilicone 11 (Gransil | Detackifying agent | 7 | 2 |
| PM gel) | | | |
| Squalane HQ (olive oil derived) (squalane) | Hydrating agent, emollient | 5 | 4 |
| Tocopheryl Acetate (Vitamin E acetate) | Hydrating agent | 6 | 0.1 |
| Polysorbate 40 (Tween 40) | Emulsifying agent | 5 | 1.5 |
| Aluminum Benzoate, mica, Disodium | Surface modifier, UV light absorber, cosmetic | 6 | 0.15 |
| Distyrybiphenyl Disulfonate, Aluminum | astringent | | |
| Chloride (Covazur Z03) | | | |
| Caprylic/capric triglyceride (crodamol GTCC) | Hydrating agent | 5 | 3 |
| Alpha Bisabolol racemic (bisabolol) | Anti-irritation agent, anti-inflammatory and | 6 | 0.02 |
| | antibacterial agent | | |
| Dipalmitoyl hydroxyproline (sepilift DPHP) | Fibroblast relaxation (reduce expression wrinkles), | 6 | 1 |
| | anti MMP, anti-elastase, synthesis of TIMP2, | | |
| | stimulates lamins, anti-oxidant, anti-inflammatory | | |
| Cyclomethicone, polysilicone-11 (Gransil | Detackifying agent | 9 | 2.1 |
| GCM) | | | |
| Glycosaminoglycans (MDI complex PF | Mmps inhibitor; skin matrix integrity, anti- | 1 | 5 |
| paraben free) | inflammatory | | |
| Hyaluronic Acid, water (hyasol BT 1%) | Hydrating agent | 1 | 2 |
| Ethylbisiminomethylguaiacol manganese | Anti-oxidant, oxygenation, DNA self-repair | 2 | 0.01 |
| chloride (Euk-134) | | | |
| Retinol (Retinol 50c liquid + polysorbate 20) | Cellular renewal | 1 | 0.003 |
| Creatine (TegoCosmo c-100) | ATP stimulation | 1 | 0.01 |
| Glycerin, butylene glycol, water, carbomer | Collagen synthesis | 1 | 0.01 |
| (emulsion stabilizing agent), polysorbate-20, | | | |
| palmitoyl pentapeptide-4 (Matrixyl 3000) | | | |
| Sesamum indicum seed oil, triticum vulgare | Anti-inflammatory agent, anti-irritation agent, | 1 | 0.03 |
| germ oil, tocopheryl acetate, caprylic/capric | hydrating agent | | |
| succinic triglyceride (LNST PF (without | | | |
| paraben)) | | | |
| Dipotassium Glycyrrhizate (Net DG) | Anti-inflammatory agent, hydrating agent | 1 | 0.02 |
| Alteromonas ferment extract, butylene glycol | Anti-irritation agent, langherhans cell protection, | 1 | 0.02 |
| (Abyssine PF) | skin matrix integrity | | |
| Imperata Cylindrica root extract, water, | Hydrating agent, osmosis protection | 1 | 3 |
| Glycerin, PEG-8, Carbomer (Moist 24) | | | |
| Ceramide 3, 6 II, 1, Phytosphingosine, | Hydrating agent, Inhibition of tyrosinase activity | 1 | 5 |
| Cholesterol, Sodium Lauroyl Lactylate, | | | |
| Carbomer, xanthan gum (sk-influx) | | | |
| Octadecenedioic Acid (O.D.A White) | Whitening agent (inhibition of Tyrosinase gene | 8 | 1.000 |
| | expression via the PPAR complex) | | |
| Caprylic/Capric triglycerides & Humulus | Whitening agent (inhibition of GM-CSF production | 8 | 2.000 |
| Lupulus Strobile (Wonderlight) | (keratinocytes)& inhibition of GSK-3beta | | |
| | (melanocytes)) | | |
| Vibrio Exopolysaccharide Extract (Exossine T) | Desquamation & cellular renewal | 1 | 2.000 |
| Water, Titanium Dioxide, Polysorbate 20, | Whitening agent (a-adrenergic antagonist receptors | 9 | 2.000 |
| Acrylates/C10-30 Alkyl Acrylate | & calcium flow regulation & stabilisation of inactive | | |
| Crosspolymer, Polymethylmethacrylate, | tyrosinase & pigment effect) | | |
| Trilaurin, Diacetiyl Boldine (Lumisphere) | | | |
| Lepidum sativum sprout extract | Whitening agent (a-MSH antagonist), Anti-oxidant | 11 | 2.000 |
| (SulforaWhite) | | | |
| Artocarpus heterophyllus seed extract | Whitening agent (reduction of melanosome | 11 | 1.000 |
| (Whitessence) | phagocytosis by keratinocytes) | | |
| Cynara Scolymus Leaf extract (Biobenefity) | Whitening agent (inhibition of Nf-kB) | 11 | 2.000 |
| | Anti-inflammatory | | |
| Uva-Ursi Leaf Extract, Magnesium Ascorbyl | Whitening agent (inhibition of tyrosinase & | 11 | 1.000 |
| Phosphate (Melfade J) | reduction of oxidation of L-DOPA) | | |
| Glycyrrhiza Glabra (Licorice Eco) | Whitening agent (inhibition of tyrosinase) | 8 | 0.290 |
| | Anti-inflammatory & Anti-oxidant | | |
| Ubiquinone (coenzyme Q10) | Cellular renewal | 1 | 0.01 |
| 1,2-hexanediol, caprylyl glycol (Symdiol 68) | Hydrating agent, preservative | 1 | 1 |
| Fragrance (Juniper Breeze #55472 | Produces or masks odours | 1 | 0.1500 |
| (Interome)) | | | |
| Glycerin, water, Centella asiatica extract, | Cellular renewal | 1 | 0.1 |
| Carica papaya extract, Iris florentina extract | | | |
| (Botamix Regenerating) | | | |
| Water (and) glycerin (and) rumex (Tyrostat- | Inhibition of tyrosinase | 3 | 0.010 |
| 11) | | | |
| Water, Glycerin, Hesperidin Methyl | Anti-elastase, vaso-regulatory, anti-oxidant, anti- | 1 | 1 |
| Chalcone, Steareth 20, Dipeptide-2, | inflammatory, anti-puffiness | | |
| Palmitoyl Tetrapeptide-7 (Eyeliss) | | | |
| Polyaminopropyl biguanide (Cosmocil CQ) | Preservative | 9 | 0.5 |
| Dehydracetic Acid (Geogard 111 A) | Preservative | 10 | 0.15 |
| | | | 100 |

### Manufacturing Process

1. In a stainless steel container, the ingredients of group 1 as identified in the Table above were mixed together with a Greaves™ Homogenizer set at 5±1 or the Greaves 2™ Homogenizer set at 1800±200 rpm until until a uniform mixture was obtained. The mixture was allowed to stand until it was incorporated at a later step. (mixture of ingredients of group 1)

2. In a coulter tank or a stainless steel container, the ingredients of group 2 as identified in the Table above were mixed together. The mixture was agitated with a manual homogenizer for 5 minutes and added to the mixture of step 1. (mixture of ingredients of groups 1-2)

3. Agitation was continued using the Greaves™ Homogenizer set at 5±1 or the Greaves 2™ Homogenizer set at 1800±200 rpm for a minimum of 20 minutes. The sample was inspected visually to ensure that it was free of undissolved brown particles and of pigments. (mixture of ingredients of groups 1-2)

4. In a small Lee™ tank, water was heated to 75±5° C. Then, while continuing the agitation with a Greaves™ homogenizer set at 5±1 or a Greaves 2™ homogenizer set at 1800±200 rpm, ingredients of group 3 were added. The mixture was mixed for a minimum of 15 minutes or until a homogenous mixture was obtained. (mixture of ingredients of group 3)

5. Then, while continuing the agitation of the mixture of step 4 with a Greaves™ homogenizer set at 5±1 or a Greaves 2™ homogenizer set at 1800±200 rpm and maintaining the temperature of the mixture at 80+/−C, the sodium carbomer was slowly added. The mixture was mixed well for a minimum of 15 minutes without exceeding 20 minutes and then the Ammonium acryloyldimet/VP copolymer (Aristoflex AVC) was slowly added (ingredients of group 4). The mixture was mixed until a homogenous mixture was obtained. (mixture of ingredients of groups 3-4)

6. In a Coulter™ tank or in a stainless steel container, the ingredients of group 5 identified in the Table above were mixed together for 5 minutes. The ingredients of group 6 identified in the Table above were then added. The mixture was maintained at 80±2° C., and while continuing the agitation, the ingredients of group 7 identified in the Table above were added and the mixture was mixed with a Greaves™ homogenizer set at 5±1 or a Greaves 2™ homogenizer set at 1800±200 rpm until a uniform mixture was obtained. (mixture of ingredients of groups 5-7).

7. When the temperature of the oily phase (step 6) reached 80±2° C. and that of the aqueous phase (step 5) reached 75±2° C., the mixture of step 6 was transferred into the mixture of step 5, while agitating. Then, the agitation was continued for a minimum of 15 minutes with the Greaves™ homogenizer set at 5±1 or the Greaves 2™ homogenizer at 1800±200 rpm. (mixture of ingredients of groups 3-7)

8. The mixture of step 7 was cooled down to 75° C. and the ingredients of group 8 were added while agitating with the Greaves™ at 3±1 or the Greaves 2™ at 1400±200 rpm. Cooling was continued to 48±2° C. while agitating with the Greaves™ at 3±1 or the Greaves 2™ at 1400±200 rpm. (mixture of ingredients of groups 3-8)

9. Using the homogenizer, the ingredients of group 9 as identified in the Table above were added to the mixture of step 8. The mixture was then mixed with the Greaves™ homogenizer at 5±1 or Greaves 2™ homogenizer at 1800±200 rpm, for a minimum de 10 minutes or until a homogeneous mixture was obtained. (mixtures of ingredients of groups 3-9)

10. The mixture of step 9 was cooled down to 38±2° C.

11. The mixture of step 3 was slowly transferred into the mixture of step 10 while agitating with the Greaves™ homogenizer set at 5±1 or Greaves 2™ homogenizer set at 1800±200 rpm. Then, agitation was continued for a minimum of 10 minutes without exceeding 20 minutes or until a smooth and homogenous mixture was obtained. The sample was inspected visually to ensure that it was free of undissolved brown particles and of pigments. (mixture of ingredients of groups 1-9)

12. In a stainless steel container, the water and Geogard 111 A were added (ingredients of group 10). The mixture was agitated with a spatula until a homogeneous mixture was obtained. (ingredients of group 10)

13. The mixture of step 12 was slowly transferred into the mixture of step 11 while agitating with the Greaves™ set at 5±1 or Greaves 2™ set at 1800±200 rpm. The ingredients of group 11 were then added while agitating with the Greaves™ homogenizer set at 5±1 or Greaves 2™ homogenizer set at 1800±200 rpm until a homogeneous mixture was obtained. The mixture was cooled down to 25±2° C. A sample of the mixture was inspected visually to ensure that it was free of brown undissolved particles and did not contain any pigments.

### Specifications

The pH was of 5.1 with typical values between 5.0 and 6.5. The viscosity calculated on a Brookfield LVT Model DV I at 25 degrees Centigrade/1 minute, spindle #4 at 6 RPMs, dial reading at 45% was of 47,000 cps with a range of between about 40,000 to 55,000 cps. The color was off white to slightly beige. The odor was typical of the fragrance used. The appearance was that of a moderately viscous cream with smooth texture.

### Accelerated Stability Testing

The testing was performed as described in Example 36 above.

The pH remained stable at all temperatures with a slight but acceptable decrease at 40° C. Viscosity decreased slightly but no liquefaction or excessive thickening was observed and no viscosity change was observed upon application on the skin. The decrease was of about 1,000 cps per month at room temperature but stabilization at 8,000 cps was expected within about 6 months to two years.

The fragrance was stable although its intensity progressively weakened over time. The fragrance started to change at 40° C. at 3 months, which could be equated, to about 2 years at 25° C. The colour at the surface of the cream darkened and the browning rate correlated to the temperature. Light did not accelerate browning. It was estimated that the cream would become beige within one year. Emulsion remained stable at all temperatures without separation or syneresis. The cream handled well sharp temperature changes. It could however only resist to one freeze-thaw cycle. With subsequent cycles, its texture was firmer and showed signs of syneresis. This cream was considered fairly stable with an expected life span of about 2 years.

### Example 38

Process for making compositions of the present invention

| Group | Ingredient |
|---|---|
| 1 | NAB Willow Bark Extract |
| 1 | HDPR |
| 1 | Water |
| 1 | Snap 8 |
| 2 | Symdiol 68 |
| 2 | Aldavine |
| 2 | Abyssine 657 |
| 2 | Abyssine PF |
| 2 | Homeo Age |
| 2 | Homeo Soothe |
| 2 | Sk-Influx |
| 2 | TegoCosmo c-100 |
| 2 | Dow corning 345 fluid |
| 2 | Lipochroman 6 |
| 2 | Net DG |
| 2 | CW (Canadian Willowherb) |
| 2 | Juniper Breeze |
| 2 | Homeo Shield |
| 2 | Matrixyl 3000 |
| 2 | MRTEX Complex |
| 2 | MDI Complex |
| 2 | Hyasol BT 1% |
| 2 | Regu Age |
| 2 | Moist 24 |
| 2 | NP Moist 24 |
| 2 | Hedione |
| 2 | Flavagrum |
| 2 | Eyeliss |
| 2 | Jeecide CAP-5 |
| 2 | 330 Regederme HS |
| 2 | Botamix Regenerating |
| 2 | Retinol 50C |
| 2 | LNST 98 |
| 2 | LNST PF |
| 2 | Sodium Ascorbyl Phosphate |
| 2 | CoQ10 |
| 2 | APT |
| 2 | Neutrazen |
| 2 | Thymulen 4 PS 100 |
| 2 | Thymulen 4BG |
| 2 | Trylagen PCB |
| 2 | Bodyfit |
| 2 | BIOSCULPTINE |
| 2 | PRO-SVELTYL |
| 2 | REMODULINE |
| 2 | SLIMACTIVE |
| 2 | SLIM FIT |
| 2 | Esculoside |
| 2 | Exossine T |
| 3 | EUK-134 |
| 3 | Glycerin |
| 4 | Dissolvine Na2 |
| 4 | Methylparaben |
| 4 | Polyoprepolymer-15 |
| 4 | PVP K-90 (Polyvinylpyrrolidone) |
| 4 | Tyrostat |
| 5 | Aristoflex AVC |
| 5 | Aristoflex HMB |
| 5 | PNC-430 |
| 5 | Potassium carbomer |
| 5 | Calcium Potassium |
| | Carboxyvinylpolymer |
| 5 | Synthalen L |
| 7 | Keltrol HP |
| 6 | Covazur ZO3 |
| 6 | Bisabolol |
| 6 | Crodamol LGE |
| 6 | Gransil DMG-6 |
| 6 | Sepilift DPHP |
| 6 | Volufiline |
| 6 | Crodamol GTCC |
| 6 | Eusolex 2292 |
| 6 | Gransil PM Gel |
| 6 | Tween 40 |
| 6 | Tween 80 |
| 6 | Lumulse 100-S |
| 6 | Propylparaben |
| 6 | Squalane |
| 6 | Vitamine E |
| 6 | Photolite PK-S |
| 6 | Titanium dioxide |
| 6 | Iron oxide yellow C33-130 4CO882 |
| 6 | O.D.A White |
| 6 | Wonderlight |
| 6 | Biobenefity |
| 6 | Licorice Eco |
| 7 | Cosmocil CQ |
| 7 | Gransil GCM |
| 7 | BPD-500/plastic powder D |
| 7 | Orgasol 2002 |
| 8 | Orange Essential oil |
| 8 | Sulphites |
| 8 | Dryline |
| 8 | Geogard 111 A |
| 8 | Lumisphere |
| 8 | SulforaWhite |
| 8 | Whitessence |
| 8 | Melfade J |

1. Ingredients of group 1 (or a selection thereof) are added gradually (e.g., one by one) in a stainless steel tank so as to enable their solubilization. Heating of about 40° C. and less than 60° C. and agitation are not required but could accelerate this step. Certain equipments may enable combinations of ingredients of group 2 with those of group 1.

2. In a stainless steel tank, with an homogenizer or any appropriate agitator, are mixed ingredients of group 2 (or a selection thereof). The mixture is homogenized until uniform mixture is achieved.

3. In a stainless steel tank, ingredients of group 3 are added: Glycerin and then Euk-134 is sifted/sprinkled. The mixture is homogenized manually for about 5 minutes and added to the mixture of step 2. This step is useful to prevent inappropriate mixing of Euk-134 but could be avoided if Euk 134 could be homogenously and gradually added to the mixture.

4. The mixture of step 1 is combined with the mixture of step 3 while agitating for about 7 minutes+/−5 minutes (depending on equipment used). The sample is visualized so as to ensure that it contains no brown undissolved particles. (mixture of ingredients of groups 1-3)

5. In a stainless steel tank, a portion of the water is heated to 75+/−10° C. While agitating (e.g., manual homogenizer), a second portion of glycerin is added with ingredients of group 4, gradually (e.g., one by one). The mixture is homogenized for at least 15 minutes+/−5 minutes (depending on equipment used) or until a homogenous mixture is obtained.

6. While maintaining homogenization and the mixture temperature at 80+/−12° C., ingredients of group 5 are slowly added to the mixture of step 5 starting with xanthan gum or carbomer. The mixture is mixed for at least 5 minutes+/−3 minutes (depending on equipment used). Then the Acryloyldimethyltaurate derivative is added slowly. The mixture is mixed until homogenous. When a xanthan gum is used, agitation is desirable to ensure a good dispersion. It could alternatively mixed with another solid (e.g., Euk-134). (mixture of ingredients of groups 4-5)

7. In a stainless steel tank, ingredients of group 6 (or a selection thereof) are added gradually (e.g., one by one). This mixture is agitated for about 2 to 12 minutes (depending on equipment used) optimally after addition of each ingredient so as to facilitate their incorporation. The mixture is then heated to 70+/−15° C. while maintaining homogenization, before adding Gransil PM gel and Gransil DMG-6. The mixture is then homogenized until obtention of a uniform mixture. The temperature, duration and agitation speed may vary depending on equipment used (surface area, material of which equipment made, heat conductivity, shape and model of agitator. etc. . . . ) and may be adjusted to achieve the desired homogeneity.

8. When the temperature of the oily phase (step 7) reaches 80±2° C. and that of the aqueous phase (step 6) reached 75±2° C., the mixture of step 7 is transferred into the mixture of step 6, while agitating. Then, the agitation is continued for a minimum of 15 minutes+/−7 minutes depending on the equipment used.

9. The agitator is then stopped and the mixture of step 8 is cooled to 34+/−7° C.

10. While homogenizing, the ingredients of group 7 are added to the mixture of step 9 gradually (e.g., one by one). Then the mixture is agitated until a homogenous mixture is obtained. (about 15 minutes+/−7 minutes depending on the equipment used).

11. The mixture of step 10 is then cooled to about 34+/−7° C.

12. The mixture of step 4 is slowly added to the mixture of step 11 while homogenizing. Homogenization is maintained for about 20 minutes or until a smooth and homogenous mixture is obtained.

13. In a stainless steal tank, a portion of water is used to dispersed Geogard 111 A. The remaining ingredients of group 8 (or a selection thereof) are added gradually (e.g., one by one) with a spatula or other equipment. This addition can also be performed directly in the tank of step 12 while it is cooling.

14. Slowly transferring the mixture of step 13 into the mixture of step 12 while homogenizing. The mixture is then cooled down to 25+/−8° C. and agitated until a smooth and homogenous mixture is obtained.

Although the present invention has been described herein above by way of specific embodiments thereof, it can be modified, without departing from the spirit and nature of the subject invention as defined in the appended claims.

1-46. (canceled)

47. An aqueous topical composition comprising: a) water; b) plural skin conditioning agents; c) at least one anti-matrix metalloproteinase agent or anti-inflammatory agent; d) at least one antioxidant or at least one DNA self-repair agent; e) at least one chelating agent; f) plural viscosity increasing agents; g) plural hydrating agents and/or moisturizers; and h) at least one emulsion stabilizer.

48. The composition of claim 47 further comprising; i) at least one cellular renewal agent; j) plural anti-irritation agents; k) at least one ATP stimulation agent; and I) at least one anti-elastase agent, vasoregulatory agent, and/or anti-puffiness agent.

49. The composition of claim 48 further comprising: m) at least one collagen synthesis agent; n) at least one osmosis protection agent; and o) at least one fragrance.

50. The composition of claim 47 further comprising: i) at least one ATP stimulation agent.

51. The composition of claim 47 further comprising: i) at least one anti-irritation agent; and j) at least one collagen synthesis agent.

52. The composition of claim 47 further comprising: i) at least one skin matrix integrity agent; j) at least one anti-angiogenesis agent; and k) at least one inhibition of tyrosinase agent and/or whitening agent.

53. An aqueous topical composition comprising: a) water; b) at least one ATP stimulation agent; c) plural skin conditioning agents and/or skin protectant agents; d) at least one anti-inflammatory agent; e) at least one antioxidant or at least one DNA self-repair agent; f) plural hydrating agents and/or moisturizers; g) at least one skin matrix integrity agent; and h) at least one fibroblast relaxation agent, lamin stimulator agent, anti-elastase agent or synthesis of TIMP2 agent.

54. The composition of claim 53 further comprising: i) plural cellular renewal agents; j) plural inhibitors of tyrosinase agents; k) at least one langherhans cell protection agent; l) plural skin matrix integrity agents; m) at least one anti-puffiness agent; n) at least one collagen synthesis agent; o) at least one delivery system to avoid irritation and inflammation; and p) at least one viscosity increasing agent.

55. The composition of claim 53 comprising: i) plural anti-inflammatory agents; j) at least one anti-angiogenesis agent; k) plural anti-irritation agents

56. The composition of claim 55 further comprising: l) plural whitening agents.

57. The composition of claim 55 further comprising: l) plural sliming agents.

58. A method for preventing or reducing a skin aging sign or a skin condition or disorder in a subject comprising applying an effective amount of the composition of claim 47 on the skin of the subject, whereby the skin aging sign or a skin condition or disorder is prevented or reduced.

59. The method of claim 58, wherein the skin aging sign is selected from the group consisting of fine lines, wrinkles, inflammation, redness, telangiectasia, skin sagging, excess sebum, enlarged pores, dark circles, loss of skin firmness, brown spot, dull skin, bags under eyes, disturbance of sebum production, loss of skin comfort, dehydration, and skin devitalization.

60. The method of claim 58, wherein the skin condition or disorder is selected from the group consisting of rosacea, acne-rosacea, spider veins, skin flushing, acne, pimples, dermatitis, rashes, irregular skin tone, cellulite, clogged pores and blotches.

61. A method for preventing or reducing a skin aging sign or a skin condition or disorder in a subject comprising applying an effective amount of the composition of claim 53 on the skin of the subject, whereby the skin aging sign or a skin condition or disorder is prevented or reduced.

62. The method of claim 61, wherein the skin aging sign is selected from the group consisting of fine lines, wrinkles, inflammation, redness, telangiectasia, skin sagging, excess sebum, enlarged pores, dark circles, loss of skin firmness, brown spot, dull skin, bags under eyes, disturbance of sebum production, loss of skin comfort, dehydration, and skin devitalization.

63. The method of claim 61, wherein the skin condition or disorder is selected from the group consisting of rosacea, acne-rosacea, spider veins, skin flushing, acne, pimples, dermatitis, rashes, irregular skin tone, cellulite, clogged pores and blotches.


SEQUENCE LISTING (1 sequence)

SEQ ID NO: 1 - protein, 5 aa, Artificial Sequence
  Synthesized
KTTKS